(19) United States
(12) Patent Application Publication
(10) Pub. No.: US 2026/0007773 A1
(43) Pub. Date: Jan. 8, 2026

(54) OCULAR VECTORS AND USES THEREOF

(71) Applicant: ALDEVRON, LLC, Fargo, ND (US)

(72) Inventors: Ricardo SALDAÑA-MEYER, Stoneham, MA (US); Jin HUH, Chapel Hill, NC (US); Jodi KENNEDY, Dedham, MA (US)

(21) Appl. No.: 18/992,141

(22) PCT Filed: Jul. 7, 2023
(86) PCT No.: PCT/US2023/069748

§ 371 Date: Jan. 7, 2025

Related U.S. Application Data
(60) Provisional application No. 63/359,125, filed on Jul. 7, 2022.
(60) Provisional application No. 63/408,353, filed on Sep. 20, 2022.

(51) Int. Cl. A61K 48/00; A61K 9/00; A61K 38/17; A61P 27/02; C12N 15/85; C12N 15/87

(52) U.S. Cl. CPC A61K 48/005; A61K 9/0048; A61K 38/1719; A61K 38/177; A61K 48/0075; A61K 48/0083; A61P 27/02; C12N 15/85; C12N 15/87

(57) ABSTRACT

Provided herein are constructs for improved expression of transgenes (e.g., ocular transgenes), nucleic acid vectors thereof, pharmaceutical compositions thereof, and methods of use thereof (e.g., methods of treatment). The expression constructs include regulatory elements such as promoters, enhancers, insulators, and intronic sequences. Expression constructs, nucleic acid vectors and pharmaceutical compositions thereof, and methods of use thereof, disclosed herein can provide effective, durable treatments for ocular diseases.

### PRIORITY

This application claims the benefit of, and claims priority to, U.S. provisional application Nos. 63/359,125 filed Jul. 7, 2022 and 63/408,353 filed Sep. 20, 2022, each of which are hereby incorporated by reference in their entireties.

### BACKGROUND

Visual impairment and blindness are a major global health concern, impacting millions of patients suffering from a wide variety of ocular pathologies. Retinal dystrophies, for example, are chronic and progressive disorders of visual function, which occur due to genetic abnormalities of retinal cellular structures (e.g., photoreceptors and/or retinal epithelial cells) and visual cycle pathways (e.g., phototransduction and visual cycle pathways required to facilitate conversion of light energy into perceptible neuronal signals). Vision impairment caused by retinal dystrophies varies from poor peripheral or night vision to complete blindness, and severity usually increases with age. Due in part to complex biological mechanisms and restricted access to the retina, safe and effective treatments for many retinal dystrophies are scarce.

While gene therapy is a promising approach for treatment of retinal dystrophies in theory, achieving and maintaining suitable expression of exogenous genes in target ocular tissues (such as cells of the retina) is a challenge. Thus, there is a need for therapeutic vectors and gene therapy methods that provide for robust and/or persistent transgene expression in the eye.

### SUMMARY

Provided herein are nucleic acid vectors having regulatory elements that confer robust (i.e., high levels of transgene expression) and/or persistent transgene expression in target cells, such as ocular cells. Such cells may include in various embodiments photoreceptor cells and/or retinal pigment epithelial cells.

In one aspect, the disclosure provides a nucleic acid vector comprising (a) a DNA sequence (e.g., a cDNA sequence) encoding ABCA4; and (b) a regulatory element comprising a sequence derived from ABCA4 intron 6. In some embodiments, the vector comprising a sequence derived from ABCA4 intron 6 placed downstream of the DNA encoding ABCA4 supports robust and/or persistent gene expression in desired cells (e.g., photoreceptor cells) and at a desired subcellular location (e.g., photoreceptor outer segment layer). In some embodiments, the regulatory element is derived from the 5′ half of ABCA4 intron 6. In some embodiments, the sequence derived from ABCA4 intron 6 comprises a nucleotide sequence that has at least 90% sequence identity to (e.g., at least 95%, at least 96%, at least 97%, at least 98%, at least 99%, or 100% identity to) at least 500 consecutive nucleotides within ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within human ABCA4 intron 6 (e.g., SEQ ID NO: 29). In some embodiments, the at least 500 consecutive nucleotides (e.g., at least 500, at least 600, at least 700, at least 800, at least 900, at least 1000, at least 1100, at least 1200, at least 1300, at least 1400, at least 1500, or at least 1600 consecutive nucleotides) comprises nucleotides within 3,158 to 4,822 of ABCA4 intron 6 (nucleotides 3,158 to 4.822 of the ABCA4 intron 6 sequence of SEQ ID NO: 29, where nucleotide 3,158 is defined as position 94,561,192 of chromosome 1, according to GRCh37/hg19) or a portion thereof. In some embodiments, the regulatory element comprises the nucleic acid sequence of SEQ ID NO: 13 or a functional variant thereof (e.g., a functional variant that has at least 90% or at least 95% or at least 97% sequence identity to SEQ ID NO: 13). In some embodiments, the regulatory element is operably linked (e.g., linked directly or indirectly via an intervening nucleotide sequence) downstream of the cDNA sequence encoding ABCA4.

In some embodiments, the nucleic acid vector further comprises a promoter operably linked upstream of the cDNA sequence encoding ABCA4. In some embodiments, the promoter comprises a native ABCA4 promoter or functional variant thereof (as described herein). In some embodiments, the native ABCA4 promoter comprises any one of SEQ ID NOs: 6-9 or a functional variant thereof as described herein. In some embodiments, the promoter comprises a cytomegalovirus (CMV) enhancer/beta-actin (CAG) promoter, an elongation factor 1 alpha (EF1A) promoter, an interphotoreceptor retinoid-binding protein (IBRP) promoter, a rhodopsin kinase (RK) promoter (e.g., a G protein-coupled receptor kinase 1; GRK1 promoter), or a functional variant thereof.

In some embodiments, the nucleic acid vector comprises, operably linked in a 5′ to 3′ direction: (a) a CAG promoter; (b) the DNA sequence (e.g., a cDNA sequence) encoding ABCA4; and (c) the regulatory element derived from the 5′ half of ABCA4 intron 6. In some embodiments, the regulatory element comprises the nucleic acid sequence of SEQ ID NO: 13 or a functional variant thereof (e.g., a functional variant that has at least 90% sequence identity to SEQ ID NO: 13). In some embodiments, the nucleic acid vector comprises, operably linked in a 5′ to 3′ direction: (a) a CAG promoter; (b) the DNA sequence (e.g., a cDNA sequence) encoding ABCA4; (c) the regulatory element derived from the S′ half of ABCA4 intron 6 (e.g., a nucleic acid sequence of SEQ ID NO: 13 or a functional variant thereof, such as a functional variant that has at least 90%, at least 91%, at least 92%, at least 93%, at least 94%, at least 95%, at least 96%, at least 97%, at least 98%, at least 99%, or 100% sequence identity to SEQ ID NO: 13; and (d) a poly(A) signal (e.g., wherein (a) is directly connected to (b) or operably linked to (b) through a nucleotide sequence, wherein (b) is directly connected to (c) or operably linked to (c) through a nucleotide sequence, and/or wherein (c) is directly connected to (d) or operably linked to (d) through a nucleotide sequence. In various embodiments, the vector is a circular vector where the 3′ end of (d) is operably linked to the 5′ of (a) by a sequence that comprises minimal bacterial DNA (e.g., no more than 200 bp or no more than 100 hp, or no more than 75 bp, or no more than 50 bp, or no more than 45 bp, no more than 40 bp, no more than 35 bp, no more than 30 bp, no more than 25 bp, no more than 20 bp of a sequence comprising bacterial nucleotide sequences. Bacterial-derived sequences in some embodiments comprise an origin of replication, which can be a minimal sequence that supports replication in E co/i. In some embodiments, the origin of replication is a truncated ColE2-P9 replication origin. In some embodiments, the nucleic acid sequence comprises the nucleotide sequence of SEQ ID NO: 26 or a functional variant thereof. For example, a functional variant may have at least 90% sequence identity to SEQ ID NO: 26 (e.g., at least 95%, at least 96%, at least 97%, at least 98%, at least 99%, or 100% identity to SEQ ID NO: 26), e.g., the nucleic acid sequence comprises at least 90% sequence identity to nucleotides 29 to 10,286 of SEQ ID NO: 26 (e.g., at least 95%, at least 96%, at least 97%, at least 98%, at least 99%, or 100% identity to nucleotides 29 to 10,286 of SEQ ID NO: 26). The sequence or functional variant thereof may express an ABCA4 protein of SEQ ID NO: 32.

In some of any of the preceding embodiments, the nucleic acid vector may further comprise an insulator sequence. In some embodiments, the insulator sequence is operably linked upstream of the DNA sequence (e.g., cDNA sequence) encoding ABCA4. In some embodiments, the insulator sequence is a chicken β-globin insulator (cHS4) comprising the nucleotide sequence of SEQ ID NO: 12. In some embodiments, the nucleic acid sequence comprises SEQ ID NO: 28 or a functional variant thereof (e.g., a functional variant that has at least 90% sequence identity to SEQ ID NO: 28). The sequence or functional variant thereof may express an ABCA4 protein of SEQ ID NO: 32.

In some of any of the preceding embodiments, the nucleic acid vector is a non-integrating DNA vector, such as a plasmid DNA vector, a minicircle DNA vector, a synthetic circular DNA vector, a closed-ended DNA vector, a doggy bone DNA vector, or a ministring DNA vector In some embodiments, the nucleic acid vector is an in vivo-produced circular DNA vector that lacks a selectable marker (e.g., drug resistance gene). In various embodiments, the vector contains no more than 200 bp of bacterially-derived sequences, or no more than 100 bp, or no more than 75 bp, or no more than 50 bp of bacterially-derived sequences. For example, the vector may comprise a truncated ColE2-P9 replication origin.

In another aspect, the invention provides a nucleic acid vector comprising: (a) a promoter comprising a native ABCA4 promoter or functional variant thereof; (b) a sequence encoding ABCA4; and (c) a regulatory element. In some embodiments, the promoter (a), the sequence encoding ABCA4 (b), and the regulatory element (c) are operably linked in a 5′ to 3′ direction. In some embodiments, the regulatory element comprises a scaffold/matrix attachment region (S/MAR) sequence. In some embodiments, the S/MAR sequence comprises an interferon-beta S/MAR sequence or a functional variant thereof. In some embodiments, the S/MAR comprises the nucleic acid sequence of SEQ ID NO: 10 or 11, or a functional variant thereof (e.g., a functional variant having at least 90% sequence identity to SEQ ID NO: 10 or 11). Alternatively, the regulatory element comprises a sequence derived from ABCA4 intron 6. In some embodiments, the regulatory element is derived from the 5′ half of ABCA4 intron 6 In some embodiments, the sequence derived from ABCA4 intron 6 comprises a nucleotide sequence that has at least 90% sequence identity to (e.g., at least 95%, at least 96%, at least 97%, at least 98%, at least 99%, or 100% identity to) at least 500 consecutive nucleotides within ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 29). In some embodiments, the at least 500 consecutive nucleotides (or more as described above) comprise nucleotides within 3,158 to 4,822 of ABCA4 intron 6. In some embodiments, the regulatory element comprises the nucleic acid sequence of SEQ ID NO: 13 or a functional variant thereof (e.g., a functional variant having at least 90% sequence identity to SEQ ID NO: 13).

In some embodiments, the nucleic acid vector further comprises an insulator sequence, e.g., an insulator sequence operably linked upstream of the cDNA sequence encoding ABCA4 (e.g., and upstream of a promoter). In some embodiments, the insulator sequence is a chicken β-globin insulator (cHS4) comprising the nucleotide sequence of SEQ ID NO: 12. In some embodiments, the nucleic acid vector lacks an origin of replication (or comprises a minimal origin) and a drug resistance gene. In such embodiments, the vector comprises no more than 200 bp of bacterially-derived sequences, or no more than 100 bp, or no more than 75 bp, or no more than 50 bp of bacterially-derived sequences. In some embodiments, the nucleic acid vector is a plasmid DNA vector, a minicircle DNA vector, a synthetic circular DNA vector, a closed-ended DNA vector, a doggybone DNA vector, or a ministring DNA vector. In some embodiments, the nucleic acid vector is an in vivo-produced circular DNA vector that lacks a selectable marker (e.g., drug resistance gene). In some embodiments, the DNA vector comprises an origin of replication such as a truncated ColE2-P9 replication origin.

In another aspect, provided is a nucleic acid vector comprising: (a) a first regulatory element comprising an insulator sequence; and (b) an ocular gene-encoding sequence. In some embodiments, the insulator sequence is operably linked 5′ to the ocular gene-encoding sequence, and upstream of a promoter. In some embodiments, the insulator sequence is a chicken β-globin insulator (cHS4). In some embodiments, the cHS4 comprises the nucleotide sequence of SEQ ID NO: 12. In some embodiments, the nucleic acid vector further includes a promoter between the insulator sequence and the ocular gene-encoding sequence. In some embodiments, the promoter is a native promoter of the ocular gene or a functional variant thereof in some embodiments, the promoter comprises, or consists of, a CAG promoter, an EF1A promoter, an IBRP promoter, an RK promoter, or a functional variant thereof.

In some embodiments, the nucleic acid vector comprises a second regulatory element. In sone embodiments, the second regulatory element is a scaffold/matrix attachment region (S/MAR) sequence, e.g., an S/MAR sequence operably linked 3′ to the ocular gene-encoding sequence. In some embodiments, the S/MAR sequence comprises an interferon-beta S/MAR sequence or a functional variant thereof. In some embodiments, the S/MAR comprises the nucleotide sequence of SEQ ID NO: 10 or 11, or a functional variant thereof.

In some embodiments, the second regulatory element comprises a sequence derived from intron 6 of ABCA4. In some embodiments, the second regulatory element is derived from the 5′ half of ABCA4 intron 6. In some embodiments, the sequence derived from ABCA4 intron 6 comprises a nucleotide sequence that has at least 90% sequence identity to (e.g., at least 95%, at least 96%, at least 97%, at least 98%, at least 99%, or 100% identity to) at least 500 consecutive nucleotides within ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within human ABCA4 intron 6 (e.g., SEQ ID NO: 29). In some embodiments, the at least 500 consecutive nucleotides are within 3,158 to 4,822 of ABCA4 intron 6. In some embodiments, the regulatory element comprises the nucleic acid sequence of SEQ ID NO: 13 or a functional variant thereof (e.g., a functional variant having at least 90% sequence identity to SEQ ID NO: 13).

In some embodiments, the ocular gene is ABCA4. In some embodiments, the ocular gene is ABCA4 and the promoter comprises a native ABCA4 promoter or functional variant thereof. In some embodiments, the promoter comprises any one of SEQ ID NOs: 6-9, or a functional variant as described herein.

In other embodiments, the ocular gene is MYO7A. In some embodiments, the ocular gene is MYO7A and the promoter comprises a native MYO7A promoter or functional variant thereof. In some embodiments, the promoter comprises any one of SEQ ID NOs: 3-5 or a functional variant described herein.

In some embodiments, the nucleic acid vector is a non-integrating DNA vector. In some embodiments, the nucleic acid vector is a plasmid DNA vector, a minicircle DNA vector, a synthetic circular DNA vector, a closed-ended DNA vector, a doggybone DNA vector, or a ministring DNA vector. In some embodiments, the nucleic acid vector is an in vivo-produced circular DNA vector that lacks a selectable marker (e.g., drug resistance gene). In some embodiments, the circular DNA vector comprises no more than 200 bp of bacterially-derived sequences, or no more than 100 bp, or no more than 75 bp, or no more than 50 bp of bacterially-derived sequences. In some embodiments, the DNA vector comprises an origin of replication, such as a truncated ColE2-P9 replication origin.

In another aspect, the invention provides a nucleic acid vector comprising: (a) a promoter comprising any one of SEQ ID NOs: 3-5 (or a functional variant as described herein); and (b) a sequence encoding MYO7A. In some embodiments, the nucleic acid further comprises a regulatory element that enhances the expression and/or persistence of MYO7A (e.g., in retinal cells). In some embodiments, the regulatory element comprises a scaffold/matrix attachment region (S/MAR) sequence. In some embodiments, the S/MAR sequence is an interferon-beta S/MAR sequence or a functional variant thereof. In some embodiments, the S/MAR comprises the nucleic acid sequence of SEQ ID NO: 10 or 11, or a functional variant as described herein.

In some embodiments, the regulatory element comprises a sequence derived from ABCA4 intron 6. In some embodiments, the regulatory element is derived from the 5′ half of ABCA4 intron 6. In some embodiments, the sequence derived from ABCA4 intron 6 comprises a nucleotide sequence that has at least 90% sequence identity to (e.g., at least 95%, at least 96%, at least 97%, at least 98%, at least 99%, or 100% identity to) at least 500 consecutive nucleotides within ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 29). In some embodiments, the at least 500 consecutive nucleotides are within nucleotides 3,158 to 4,822 of ABCA4 intron 6. In some embodiments, the regulatory element comprises the nucleic acid sequence of SEQ ID NO: 13 or a functional variant thereof (e.g., a functional variant having at least 900% sequence identity to SEQ ID NO: 13, or as described herein).

In some embodiments, the nucleic acid vector further includes an insulator sequence. In some embodiments, the insulator sequence is upstream of the cDNA sequence encoding MYO7A. In some embodiments, the insulator sequence comprises SEQ ID NO: 12 or a functional variant as described herein.

In some embodiments, the nucleic acid vector is a non-integrating DNA vector. In some embodiments, the nucleic acid vector is a plasmid DNA vector, a minicircle DNA vector, a synthetic circular DNA vector, a closed-ended DNA vector, a doggybone DNA vector, or a ministring DNA vector. In some embodiments, the nucleic acid vector is an in vivo-produced circular DNA vector that lacks a selectable marker (e.g., drug resistance gene) and/or a recombination site. In some embodiments, the circular vector comprises no more than 200 bp of bacterially-derived sequences, or no more than 100 hp, or no more than 75 bp, or no more than 50 bp of bacterially-derived sequences. In some embodiments, the DNA vector comprises an origin of replication, such as a truncated ColE2-P9 replication origin.

In another aspect, this disclosure provides a circular DNA vector comprising (a) a therapeutic protein-encoding sequence; and (b) a scaffold/matrix attachment region (S/MAR) sequence, wherein the circular DNA vector comprises no more than 200 bp of bacterially-derived sequences, or no more than 100 bp, or no more than 75 bp, or no more than 50 bp of bacterially-derived sequences, and which include a minimal origin of replication. In some embodiments, the circular DNA vector comprises an origin of replication, such as a truncated ColE2-P9 replication origin. Such vectors allow for production of circular DNA in bacteria with minimal bacterial-derived sequences in the final vector.

In some embodiments, the synthetic circular DNA vector further includes a promoter, e.g., wherein the therapeutic protein-encoding sequence and the S/MAR are operably linked 3′ to the promoter. In some embodiments, the S/MAR sequence is an interferon-beta S/MAR sequence or a functional variant thereof. In some embodiments, the S/MAR sequence is a full-length interferon-beta S/MAR sequence comprising the nucleotide sequence of SEQ ID NO: 10, or a functional variant thereof. In some embodiments, the S/MAR sequence is an interferon-beta S/MAR variant sequence comprising the nucleotide sequence of SEQ ID NO: 11, or a functional variant thereof.

In some embodiments, the promoter is a native promoter of the ocular gene or functional variant thereof. In some embodiments, the promoter is a constitutive promoter. In some embodiments, the promoter comprises a CAG promoter, an EF1A promoter, an IBRP promoter, an RK promoter, or a functional variant thereof.

In some embodiments, the S/MAR sequence is flanked by a 5′ splice donor site and a 3′ splice acceptor site. In some embodiments, the S/MAR sequence is located downstream of the therapeutic protein-encoding sequence.

In some embodiments, the synthetic circular DNA vector further comprises a poly-A sequence downstream of the therapeutic protein-encoding sequence and the S/MAR sequence.

In some embodiments, the ocular gene is MYO7A. In some embodiments, the promoter is a native MYO7A promoter or functional variant thereof. In some embodiments, the native MYO7A promoter or functional variant thereof comprises the nucleotide sequence of any one of SEQ ID NOs: 3-5.

In some embodiments, the ocular gene is ABCA4. In some embodiments, the promoter is a native ABCA4 promoter or functional variant thereof. In some embodiments, the native ABCA4 promoter or variant thereof comprises the nucleotide sequence of any one of SEQ ID NOs: 6-9.

In another aspect, provided is a nucleic acid vector comprising: (a) a promoter comprising the nucleotide sequence of any one of SEQ ID NOs: 6-9 or a functional variant thereof (e.g., a functional variant having at least 90% identity to SEQ ID NO: 6, 7, 8, or 9), and (b) a sequence encoding ABCA4. In some embodiments, the nucleic acid vector further comprises a scaffold/matrix attachment region (S/MAR) sequence downstream of the promoter and/or the sequence encoding ABCA4. In some embodiments, the S/MAR sequence comprises an interferon-beta S/MAR sequence or a functional variant thereof. In some embodiments, the S/MAR sequence is a full-length interferon-beta S/MAR sequence comprising the nucleotide sequence of SEQ ID NO: 10 or is an interferon-beta S/MAR variant sequence comprising the nucleotide sequence of SEQ ID NO: 11.

In some embodiments, the nucleic acid vector comprises a regulatory element comprising a sequence derived from ABCA4 intron 6. In some embodiments, the regulatory element is derived from the 5′ half of ABCA4 intron 6. In some embodiments, the sequence derived from ABCA4 intron 6 comprises a nucleotide sequence that has at least 90% sequence identity to (e.g., at least 95%, at least 96%, at least 97%, at least 98%, at least 99%, or 1000% identity to) at least 500 consecutive nucleotides within ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 29). In some embodiments, the at least 500 consecutive nucleotides within nucleotides 3,158 to 4,822 of ABCA4 intron 6. In some embodiments, the regulatory element comprises the nucleic acid sequence of SEQ ID NO: 13 or a functional variant thereof (e.g., a functional variant having at least 90% sequence identity to SEQ ID NO: 13). In some embodiments, the regulatory element is downstream of the sequence encoding ABCA4.

In some embodiments, the nucleic acid vector further comprises an insulator sequence. In some embodiments, the insulator sequence is operably linked 5′ to the cDNA sequence encoding ABCA4. In some embodiments, the insulator sequence is a chicken β-globin insulator (cHS4) comprising SEQ ID NO: 12.

In another aspect, this disclosure provides a pharmaceutical composition comprising a therapeutically effective amount of the nucleic acid vector of any aspect or embodiment of this disclosure, and a pharmaceutically acceptable carrier. In some embodiments, the nucleic acid vector is nonviral. In some embodiments, the nucleic acid vector is a naked vector. In some embodiments, the nucleic acid vector is formulated as a liposomal or nanoparticulate formulation. In some embodiments, the pharmaceutical composition is formulated for ocular administration (e.g., subretinal, intravitreal, or suprachoroidal administration).

In another aspect, this disclosure provides methods for expressing a transgene in a subject (e.g., in the eye of a subject) (and method for treating an ocular disorder) by administering to the subject the nucleic acid vector or pharmaceutical composition of any of the preceding embodiments of any of the preceding aspects. In some embodiments, the subject has an ocular disorder (e.g., an ocular disorder associated with the transgene). In some embodiments, the ocular disorder is a Mendelian-heritable retinal dystrophy. In some embodiments, the ocular disorder is Stargardt disease, rod cone dystrophy, retinitis pigmentosa, or Usher syndrome. In some embodiments, the nucleic acid vector is delivered by in vivo electroporation. In various embodiments, in vivo electroporation may comprise: (a) contacting an electrode to an interior region of an eye of the subject, wherein an extracellular space in the retina of the eye comprises a nucleic acid vector or synthetic circular DNA vector of any embodiments described above; and (b) while the electrode is contacting the interior region of the eye, transmitting one or more pulses of electrical energy through the electrode aw conditions suitable for electrotransfer of the nucleic acid vector into target retinal cells. In some embodiments, the interior region of the eye contacting the electrode comprises the vitreous humor. In some embodiments, the electrode is within 10 mm of the retina upon transmission of the one or more pulses of electrical energy. In some embodiments, the interior region of the eye contacting the electrode comprises the retina. In some embodiments, the interior region of the eye contacting the electrode comprises the subretinal space. In some embodiments, the conditions suitable for electrotransfer into the target retinal cell comprise a field strength at the target retinal cell from 10 V/cm to 1,500 V/cm. In some embodiments, 1 to 12 pulses of electrical energy are transmitted. In some embodiments, the total number of pulses of electrical energy are transmitted within 1 to 20 seconds. In some embodiments, the pulses of electrical energy are square waveforms. In some embodiments, the pulses of electrical energy have an amplitude from 5 V to 250 V. In some embodiments, each of the pulses of electrical energy is from 10 to 200 milliseconds in duration.

In various embodiments, the methods of the disclosure this disclosure express ABCA4 in the eye of a subject. In some embodiments, the ABCA4 expressed by the nucleic acid vector is expressed in the photoreceptors, e.g., outer layer segment of the photoreceptors. In some embodiments, the ABCA4 expressed by the nucleic acid vector is expressed to a higher level in the photoreceptor outer layer segment of the eye than in the retinal pigment epithelial layer. In some embodiments, the subject has an ABCA4-associated retinal dystrophy (e.g., Stargardt disease, rod cone dystrophy, or retinitis pigmentosa).

In some embodiments, the methods of this disclosure express MYO7A in the eye of a subject. In some embodiments, the subject has a MYO7A-associated retinal dystrophy. In some embodiments, the MYO7A-associated retinal dystrophy is Usher syndrome.

Other aspects and embodiments of this disclosure will be apparent from the following detailed description and working examples.

### INCORPORATION BY REFERENCE

Each patent, publication, and non-patent literature cited in the application is hereby incorporated by reference in its entirety as if each was incorporated by reference individually. To the extent publications and patents or patent applications incorporated by reference conflict with the disclosure contained in the specification, the specification is intended to supersede and/or take precedence over any such conflicting material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which.

FIG. 1 shows representative human ABCA4 therapeutic expression construct designs.

FIG. 2 shows representative human ABCA4 therapeutic expression constructs with native promoter elements identified by mapping ABCA4 chromatin regulatory elements using ChIP-Seq for H3K27ac and ATAC-Seq in the indicated cell types (fetal retinal pigment epithelium cells (Fetal RPE); induced pluripotent stem cell retinal pigment epithelium cells (iPSC RPE)).

FIG. 3 shows the transfection efficiency of various human ABCA4 expression constructs as assessed by relative DNA copy number compared to a genomic locus region.

FIG. 4 shows the mRNA expression of ABCA4 normalized to DNA copy number, induced by selected ABCA4 expression constructs, with the most effective constructs denoted with an *.

FIG. 5 shows representative human Myosin VIIA (MYO7A) therapeutic expression construct designs with native promoter elements identified by mapping MYO7A chromatin regulatory elements using ChIP-Seq for H3K27ac and ATAC-Seq in the indicated cell types (fetal retinal pigment epithelium cells (Fetal RPE); induced pluripotent stem cell retinal pigment epithelium cells (iPSC RPE)).

FIG. 6 shows the results of MYO7A expression construct transfection experiments in iRPE cells.

FIG. 7 shows the relative GFP and mRNA expression of MYO7A in the retinal pigment epithelium/choroid and neural retina (NR) layers of a pig eye, following subretinal delivery of selected expression constructs using cellular delivery of genetic material by in vivo electrotransfer.

FIG. 8 shows the effects of including interferon-β scaffold matrix attachment region (S/MAR) sequences in expression constructs after 19 days as assessed by fluorescence activated cell sorting (FACS), and qPCR.

FIGS. 9A-9D are photomicrographs showing histology of an adult pig eye after in vivo electrotransfer of a circular DNA vector encoding GFP FIG. 9A shows single staining with ABCA4 in blue. FIG. 9B shows staining with ABCA4 in blue and rhodopsin in yellow. FIG. 9C shows staining with ABCA4 in blue and Opsin in yellow. FIG. 9D shows staining with ABCA4 in blue and RPE65 in yellow.

### DETAILED DESCRIPTION

The present invention provides constructs (e.g., nucleic acid vectors) for improved expression of transgenes (e.g., ocular transgenes for expression in the eye), pharmaceutical compositions thereof, and methods of use thereof (e.g., methods of treatment). The invention is based, at least in part, on the development of regulatory elements that affect transgene expression and/or persistence of expression constructs that include the regulatory elements. Regulatory elements of the expression constructs can include promoters, enhancers, insulators, and other regulatory elements that can enhance expression and/or persistence in target ocular cells. Expression constructs, nucleic acid vectors and pharmaceutical compositions thereof, and methods of use thereof, disclosed herein can provide effective, durable treatments for ocular diseases.

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the term “expression construct” refers to a nucleic acid sequence (e.g., DNA sequence) that is expressed by a cell upon delivery to the cell, e.g., by a nucleic acid vector containing the expression construct. An expression construct may include a sequence of interest (e.g., one or more transgenes, e.g., therapeutic transgenes) and regulatory elements operably linked thereto (e.g., promoters, S/MARs, intronic sequences, insulators, etc.) which can enhance expression and/or persistence of the DNA vector in a target cell.

As used herein, the terms “vector” and “nucleic acid vector” are used interchangeably and refer to a nucleic acid molecule capable of delivering a therapeutic sequence to which it is linked into a target cell in which the therapeutic sequence can then be transcribed, replicated, processed, and/or expressed in the target cell. One type of vector is a “plasmid”, which refers to a circular double stranded DNA loop containing a bacterial backbone into which additional DNA segments may be ligated. Another type of vector is a phage vector. Another type of vector is a viral vector (e.g., adeno-associated viral (AAV) vector), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”). Any of the nucleic acid vectors described herein may be referred to as “isolated nucleic acid vectors.”

As used herein, the term “circular DNA vector” refers to a DNA vector in a circular form. Such circular form can be capable (in some embodiments) of being amplified into concatemers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” is used interchangeably herein with the terms “covalently closed and circular DNA vector” and “C³DNA.” A skilled artisan will understand that such circular vectors include vectors that are covalently closed with supercoiling and complex DNA topology. In particular embodiments, a circular DNA vector is supercoiled (e.g., monomeric supercoiled). In other embodiments, a circular DNA vector is relaxed open circular (covalently closed without supercoiling).

The term “synthetic,” as used herein, describes a vector (e.g., circular DNA vector) that was produced in a cell-free process in which bacterial cells are absent from their production from templates. Exemplary cell-free processes for producing synthetic circular DNA vectors are provided in International Patent Publication WO 2019/178500, which is hereby incorporated by reference in its entirety.

As used herein, the term “therapeutic sequence” refers to the portion of a DNA molecule (e.g., a DNA vector) that contains genetic material required for transcription in a target cell of one or more therapeutic moieties, which may include one or more coding sequences, promoters, terminators, introns, and/or other regulatory elements. A therapeutic moiety can be a therapeutic protein (e.g., a replacement protein, such as a protein that replaces a defective protein in the target cell) and/or a therapeutic nucleic acid (e.g., one or more microRNAs). In DNA vectors having more than one transcription unit, the therapeutic sequence contains the plurality of transcription units. A therapeutic sequence may include one or more genes (e.g., heterologous genes or transgenes, which may be ocular genes) to be administered for a therapeutic purpose. In some embodiments, the therapeutic sequence is a mammalian sequence (e.g., a human sequence).

As used herein, an “ocular gene” means a gene that is preferentially, selectively, or exclusively expressed in ocular tissue or that is involved in ocular functions such as eyesight.

As used herein, the term “therapeutic protein” refers to a protein that can treat a disease or disorder in a subject. In some embodiments, a therapeutic protein is a therapeutic replacement protein administered to replace a defective (e.g., mutated) protein in a subject. In some embodiments, a therapeutic protein is the same or functionally similar to a native protein that is not defective in a subject.

As used herein, the term “therapeutic replacement protein” refers to a protein that is structurally similar to (e.g., structurally identical to) a protein that is endogenously expressed by a normal (e.g., healthy) individual. A therapeutic replacement protein can be administered to an individual that suffers from a disorder associated with a dysfunction of (or lack of) the protein to be replaced. In some embodiments, the therapeutic replacement protein corrects a defect in a protein resulting from a mutation (e.g., a point mutation, an insertion mutation, a deletion mutation, or a splice variant mutation) in the gene encoding the protein. Therapeutic replacement proteins do not include non-endogenous proteins, such as proteins associated with a pathogen (e.g., as part of a vaccine). Therapeutic replacement proteins may include enzymes, growth factors, hormones, interleukins, interferons, cytokines, anti-apoptosis factors, anti-diabetic factors, coagulation factors, anti-tumor factors, liver-secreted proteins, or neuroprotective factors. In some instances, the therapeutic replacement protein is monogenic.

As used herein, the term “backbone sequence” refers to a portion of plasmid DNA outside the therapeutic sequence that includes one or more bacterial origins of replication or fragments thereof, one or more drug resistance genes or fragments thereof, one or more recombination sites, or any combination thereof. In some embodiments, the backbone sequence includes one or more bacterial origins of replication. Backbone sequences include truncated plasmid backbones of 200 base pairs or less (e.g., 100 base pairs or less, or 50 bp or less), which may include, e.g., a functional origin of replication.

As used herein, the term “recombination site” refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “flank,” “flanking,” and “flanked” refer to a pair of regions or points on a nucleic acid molecule (e.g., a plasmid DNA vector) that are outside a reference region of the nucleic acid molecule. In some embodiments, a pair of regions or points flanking a reference region on a nucleic acid are adjacent to (i.e., abut) the reference region (i.e., there are no intervening bases between the reference point and the flanking point). In other embodiments, a pair of regions or points on a nucleic acid molecule that flank a reference region are separated from the reference region by one or more intervening bases (e.g., up to 1,000 intervening bases).

As used herein, the term “operably linked” refers to an arrangement of elements, wherein the components so described are configured so as to perform their usual function. A nucleic acid is “operably linked” when it is placed into a functional relationship with another nucleic acid sequence. For example, a promoter is operably linked to one or more heterologous genes if it affects the transcription of the one or more heterologous genes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence, and the promoter sequence can still be considered “operably linked” to the coding sequence.

As used herein, the terms “scaffold/matrix attachment region” and “S/MAR” each refers to a DNA sequence of at least 200 nucleotides which mediates attachment of the DNA to a nuclear matrix of a eukaryotic cell, wherein the DNA sequence has at least three sequence motifs ATTA per 100 nucleotides over a stretch of at most 200 nucleotides. Exemplary S/MAR sequences are described in Liebich et al., Nucleic Acids Res. 2002, 30.312-374 and in international Patent Publication No. WO 2019/060253, the S/MAR descriptions of each of which are hereby incorporated by reference.

The term “MYO7A” refers to any native MYO7A (also known as DFNB2, MYU7A, NSRD2, USH1B, DFNA11, or MYOVIIA) from any vertebrate source, including mammals such as primates (e.g., human and cynomolgus monkeys) and rodents (e.g., mice and rats), unless otherwise indicated, as well as functional variants (e.g., natural or synthetic variants), e.g., mutants, muteins, analogs, subunits, receptor complexes, isotypes, splice variants, and fragments thereof. Functional variants can be determined on the basis of known MYO7A signaling. MYO7A encompasses full-length, unprocessed MYO7A, as well as any form of MYO7A that results from native processing in the cell. An exemplary human MYO7A sequence is provided as National Center for Biotechnology Information (NCBI) Gene ID: 4647. In some instances, the MYO7A is encoded by a sequence having at least 95% sequence identity to SEQ ID NO: 2 or SEQ ID NO: 45 (e.g., at least 96%, at least 97%, at least 98%, at least 99% or 100% sequence identity to SEQ ID NO: 2 or SEQ ID NO: 45). In various embodiments, MYO7A constructs encode a protein comprising the amino acid sequence of SEQ ID NO: 31.

The term “ABCA4” refers to any native ABCA4 from any vertebrate source, including mammals such as primates (e.g., human and cynomolgus monkeys) and rodents (e.g., mice and rats), unless otherwise indicated, as well as functional variants (e.g., natural or synthetic variants), e.g., mutants, muteins, analogs, subunits, receptor complexes, isotypes, splice variants, and fragments thereof. Functional variants can be determined on the basis of known ABCA4 signaling. ABCA4 encompasses full-length, unprocessed ABCA4, as well as any form of ABCA4 that results from native processing in the cell. An exemplary human ABCA4 sequence is provided as NCBI Reference Sequence: NG_009073 or NM_000350. In some instances, the ABCA4 is encoded by a nucleotide sequence having at least 95% sequence identity to SEQ ID NO: 1 or SEQ ID NO: 44 (e.g., at least 96%, at least 97%, at least 98%, at least 99% or 100% sequence identity to SEQ ID NO: 1 or SEQ ID NO: 44). In various embodiments, the ABCA4 constructs encode a protein comprising the amino acid sequence of SEQ ID NO: 32.

As used herein, the term “ABCA4 intron 6” refers to a native nucleic acid sequence beginning from the nucleotide directly 3′ (i.e., downstream) to the 3′ end of ABCA4 exon 6 and ending on the nucleotide directly 5′ (i.e., upstream) to the 5′ end of ABCA4 exon 7. An exemplary sequence of a native human ABCA4 introit 6 is given by SEQ ID NO: 29. As used herein, nucleotide numbering of human ABCA4 intron 6 begins at the first position of intron 6 according to NG_009073; i.e., nucleotide 1 of ABCA4 intron 6 corresponds to chromosome 1, strand (−), position 94,564,349 according to GRCh37/hg19. For example, nucleotide 3,158 of ABCA4 intron 6 corresponds to GRCh37/hg19 position 94,561,192 of chromosome 1, strand (−).

The terms “regulatory element” and “control element” are used interchangeably herein and each refer to a non-coding nucleic acid region, such as a promoter, enhancer, and silencer, which function to affect gene expression (e.g., level of expression and/or persistence of expression). In some embodiments, a regulatory element is not transcribed into mRNA. In other embodiments, a regulatory element is transcribed into mRNA but not translated into protein. Suitable regulatory elements are described in International Publication No. WO 2021/055760, which is hereby incorporated by reference in its entirety.

A regulatory element is “derived” from a reference sequence (e.g., a native intron) when it contains a functional sequence, or functional variant of a sequence, contained within the reference sequence. A regulatory element derived from a reference sequence need not have the same level of function as the reference sequence; the functional sequence of the regulatory element must confer a detectable or significant function (e.g., improve the level and/or persistence of expression, compared to an expression construct lacking the functional sequence of the regulatory element).

The term “promoter” refers to a regulatory element that regulates transcription of a gene operably linked thereto and includes (a) one or more sequence sufficient to direct transcription and/or (b) recognition sites for RNA polymerase and other transcription factors required for efficient transcription. In some embodiments, the promoter is operably linked 5′ to the gene (e.g., operably linked upstream of the gene). Some promoters can direct cell-specific expression.

As used herein, the term “naked” refers to a nucleic acid molecule (e.g., a circular DNA vector) that is not encapsulated in a lipid envelope (e.g., a liposome) or a polymer matrix and is not physically associated with (e.g., covalently or non-covalently bound to) a solid structure (e.g., a particulate structure) upon administration to the individual. In some instances of the present invention, a pharmaceutical composition includes a naked circular DNA vector.

As used herein, the term “isolated” means artificially produced and not integrated into a native host genome. For example, isolated nucleic acid vectors include nucleic acid vectors that are naked, as well as those that are encapsulated in a lipid envelope (e.g., a liposome) or a polymer matrix. In some embodiments, the term “isolated” refers to a DNA vector that is: (i) synthetic, e.g., amplified in vitro (e.g., in a cell-free environment), for example, by rolling-circle amplification or polymerase chain reaction (PCR); (ii) recombinantly produced by molecular cloning; (iii) replicated in host cells and recovered by at least partial purification; or (iv) synthesized by, for example, chemical synthesis. An isolated nucleic acid vector is one which is readily manipulable by recombinant DNA techniques well-known in the art. Thus, a nucleotide sequence contained in a vector in which 5′ and 3′ restriction sites are known or for which PCR primer sequences have been disclosed is considered isolated, but a nucleic acid sequence existing in its native state in its natural host is not. An isolated nucleic acid vector may be substantially purified, but need not be.

As used herein, the terms “individual” and “subject” are used interchangeably and include any mammal in need of treatment or prophylaxis, e.g., by a therapeutic circular DNA vector, or pharmaceutical composition thereof, described herein. In some embodiments, the individual or subject is a human. In other embodiments, the individual or subject is a non-human mammal (e.g., a non-human primate such as a monkey, a mouse, a pig, a rabbit, a cat, or a dog). The individual or subject may be male or female.

As used herein, an “effective amount” or “effective dose” of a DNA vector, or pharmaceutical composition thereof, refers to an amount sufficient to achieve a desired biological, pharmacological, or therapeutic effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response, relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, therapeutic circular DNA vectors of the invention are used to delay development of a disease or to slow the progression of a disease (e.g., retinal degeneration).

As used herein, a “target cell” refers to a cell that expresses a therapeutic protein encoded by a therapeutic gene. In some embodiments, a target cell is a retinal cell. For example, in particular embodiments, a target cell is a retinal pigment epithelial (RPE) cell. In other embodiments, a target cell is a photoreceptor. In particular embodiments, RPE cells and photoreceptors are target cells.

As used herein, “delivering,” “to deliver,” and grammatical variations thereof, means causing an agent (e.g., a DNA vector) to access a target cell. The agent can be delivered by administration of the agent to an individual having the target cell (e.g., systemically or locally administering the agent) such that the agent gains access to the organ or tissue in which the target cell resides (e.g., retina). Additionally, or alternatively, the agent can be delivered by applying a stimulus to a tissue or organ harboring the agent, wherein the stimulus causes the agent to enter the target cell. Thus, in some instances, an agent is delivered to a target cell by transmitting an electric field into a tissue harboring the agent at conditions suitable for electrotransfer of the agent into a target cell within the tissue.

As used herein, “electrotransfer” refers to movement of a molecule (e.g., a nucleic acid vector, such as a naked nucleic acid vector) across a membrane of a target cell (e.g., from outside to inside the target cell, such as a retinal cell) that is caused by transmission of an electric field (e.g., a pulsed electric field) to the microenvironment in which the cell resides (e.g., retina). Electrotransfer may occur as a result of electrophoresis, i.e., movement of the molecule along an electric field, based on a charge of the molecule. Electrophoresis can induce electrotransfer, for example, by moving the molecule into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, e.g., generation of pores in the target cell caused by transmission of an electric field (e.g., a pulsed electric field), wherein the size, shape, and duration of the pores are suitable to accommodate movement of the molecule from outside the target cell to inside the target cell. Thus, in some instances, electrotransfer occurs as a result of a combination of electrophoresis and electroporation.

As used herein, “administering” is meant a method of giving a dosage of an agent of this disclosure or a composition thereof to an individual.

The terms “level of expression” or “expression level” are used interchangeably and generally refer to the amount of a polynucleotide or an amino acid product or protein in a biological sample (e.g., retina). “Expression” generally refers to the process by which gene-encoded information is converted into the structures present and operating in the cell. Therefore, according to the invention, “expression” may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a therapeutic sequence, or a functional portion thereof (e.g., one or more coding sequences of a therapeutic DNA vector), is expressible in the cell in which it was transfected (“intra-cellular persistence”) or any progeny of the cell in which it was transfected (“trans-generational persistence”) A therapeutic sequence, or functional portion thereof, may be expressible if it is not silenced, e.g., by DNA methylation and/or histone methylation and compaction. Expression persistence can be assessed by detecting or quantifying (i) mRNA transcribed from the therapeutic sequence in the target cell or progeny thereof (e.g., through qPCR, RNA-seq, or any other suitable method) and (ii) protein translated from the therapeutic sequence in the target cell or progeny thereof (e.g., through Western blot, ELISA, or any other suitable method). In some instances, expression persistence is assessed by detecting or quantifying therapeutic DNA in the target cell or progeny thereof (e.g., the presence of therapeutic circular DNA vector in the target cell, e.g., through episomal DNA copy number analysis) in conjunction with either or both of (i) mRNA transcribed from the therapeutic sequence in the target cell or progeny thereof and (ii) protein translated from the therapeutic sequence in the target cell or progeny thereof. Expression persistence of a therapeutic sequence, or a functional portion thereof, can be quantified relative to a reference vector, such as a control vector produced in bacteria and having one or more bacterial signatures not present in the vector of the invention (e.g., a plasmid), using any gene expression characterization method known in the art. Expression persistence can be quantified at any given time point following administration of the vector. For example, in some embodiments, expression of a DNA vector of this disclosure persists for at least two weeks after administration if it is detectable in the target cell, or progeny thereof, two weeks after administration of the DNA vector. In some embodiments, expression of a DNA vector “persists” in a target cell if it is detectable in the target cell at two weeks, three weeks, four weeks, six weeks, two months, three months, four months, five months, six months, seven months, eight months, nine months, ten months, eleven months, one year, or longer after administration. In some embodiments, expression of a DNA vector is said to persist for a given period after administration if a significant fraction of the original expression level remains (e.g., at least 10%, at least 20%, at least 30%, at least 40%, at least 50%, at least 70%, or at least 100% of the original expression level) after the given period of time.

As used herein, “intra-cellular persistence” refers to the duration of time during which a therapeutic sequence, or a functional portion thereof (e.g., one or more coding sequences of a therapeutic DNA vector), is expressible in the cell in which it was transfected (e.g., a target cell, such as a post-mitotic cell or a quiescent cell). Intra-cellular persistence can be assessed by detecting or quantifying (i) mRNA transcribed from the therapeutic sequence in the target cell and (ii) protein translated from the therapeutic sequence in the target cell. In some instances, intra-cellular persistence is assessed by detecting or quantifying therapeutic DNA in the target cell in conjunction with either or both of (i) mRNA transcribed from the therapeutic sequence in the target cell and (ii) protein translated from the therapeutic sequence in the target cell. In some embodiments, a DNA vector of the invention exhibits improved intra-cellular persistence relative to a reference vector (e.g., a DNA vector containing a regulatory element exhibits improved intra-cellular persistence relative to a reference vector that does not contain the regulatory element; or a circular DNA vector exhibits improved intra-cellular persistence relative to a reference vector that is a plasmid DNA vector or contains one or more bacterial signatures not included in the circular DNA vector.

As used herein, “trans-generational persistence” refers to the duration of time during which a therapeutic sequence is expressible in progeny of the cell in which the gene was transfected (e.g., such as first-generation, second-generation, third-generation, or fourth-generation descendants of the cell in which the vector was transfected). Trans-generational persistence accounts for any dilution of a gene over cell divisions and may therefore be useful in measuring persistence of a vector in a dividing tissue over time. In some embodiments, the therapeutic circular DNA vector of the disclosure exhibits improved trans-generational persistence relative to a reference vector. Trans-generational persistence can be assessed by detecting or quantifying (i) mRNA transcribed from the therapeutic sequence in progeny of the target cell and (ii) protein translated from the therapeutic sequence in progeny of the target cell. In some instances, intra-cellular persistence is assessed by detecting or quantifying therapeutic DNA in progeny of the target cell in conjunction with either or both of (i) mRNA transcribed from the therapeutic sequence in progeny of the target cell and (ii) protein translated from the therapeutic sequence in progeny of the target cell.

As used herein, a “functional variant” of a nucleic acid sequence differs in at least one nucleic acid residue from the reference nucleic acid sequence, such as a naturally occurring nucleic acid sequence, wherein relevant functional activity of the variant is at least 90% of the level of relevant functional activity of the reference nucleic acid sequence (e.g., substantially similar to the relevant function of the reference nucleic acid sequence). In this context, the difference in at least one nucleic acid residue may consist, for example, in a mutation of an nucleic acid residue to another nucleic acid, a deletion or an insertion. A variant may encode a homolog, isoform, or transcript variant of a therapeutic protein or a fragment thereof encoded by the reference nucleic acid sequence, wherein the homolog, isoform or transcript variant is characterized by a degree of identity or homology, respectively, as defined herein.

In some instances, a functional variant of a polynucleotide or polypeptide includes at least one nucleic acid substitution (e.g., 1-100 nucleic acid or amino acid substitutions, 1-50 nucleic acid or amino acid substitutions, 1-20 nucleic acid or amino acid substitutions, 1-10 nucleic acid or amino acid substitutions, e.g., 1 nucleic acid or amino acid substitution, 2 nucleic acid or amino acid substitutions, 3 nucleic acid or amino acid substitutions, 4 nucleic acid or amino acid substitutions, S nucleic acid or amino acid substitutions, 6 nucleic acid or amino acid substitutions, 7 nucleic acid or amino acid substitutions, 8 nucleic acid or amino acid substitutions, 9 nucleic acid or amino acid substitutions, or 10 nucleic acid or amino acid substitutions). Nucleic acid substitutions that result in the expressed polypeptide having an exchanged in amino acids from the same class are referred to herein as conservative substitutions. In particular, these are amino acids having aliphatic side chains, positively or negatively charged side chains, aromatic groups in the side chains or amino acids, the side chains of which can form hydrogen bridges, e.g., side chains which have a hydroxyl function. By conservative substitution, e.g., an amino acid having a polar side chain may be replaced by another amino acid having a corresponding polar side chain, or, for example, an amino acid characterized by a hydrophobic side chain may be substituted by another amino acid having a corresponding hydrophobic side chain.

In order to determine the percentage to which two sequences (e.g., nucleic acid sequences or amino acid sequences) are identical, the sequences can be aligned in order to be subsequently compared to one another. For this purpose, gaps can be inserted into the sequence of the first sequence and the component at the corresponding position of the second sequence can be compared. If a position in the first sequence is occupied by the same component as is the case at a corresponding position in the second sequence, the two sequences are identical at this position. The percentage, to which two sequences are identical, is a function of the number of identical positions divided by the total number of positions. The percentage to which two sequences are identical can be determined using a mathematical algorithm. A preferred, but not limiting, example of a mathematical algorithm, which can be used is the algorithm of Karlin et al. (1993), PNAS USA, 90:5873-5877 or Altschul et al. (1997), Nucleic Acids Res., 25:3389-3402. Such an algorithm can be integrated, for example, in the BLAST program. Sequences which are identical to the sequences of the present invention to a certain extent can be identified by this program.

The term “pharmaceutically acceptable” means safe for administration to a mammal, such as a human. In some embodiments, a pharmaceutically acceptable composition is approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which a vector or composition of the invention is administered. Examples of suitable pharmaceutical carriers are described in “Remington's Pharmaceutical Sciences,” Mack Publishing Co., Easton, PA., 23^{rd} edition, 2020.

The terms “a” and “an” mean “one or more of.” For example. “a gene” is understood to represent one or more such genes. As such, the terms “a” and “an,” “one or more of a (or an),” and “at least one of a (or an)” are used interchangeably herein.

As used herein, the term “about” refers to a value within +10% variability from the reference value, unless otherwise specified.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as“contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

Headings provided herein are for convenience only and do not limit the scope or meaning of the claimed disclosure.

### II. Expression Constructs

Embodiments disclosed herein include expression constructs that provide for expression of a transgene, such as a therapeutic sequence by a nucleic acid vector. Exemplary vectors can be selected from a nonviral DNA vector (e.g., a naked DNA vector), a circular DNA vector (e.g., a supercoiled circular DNA vector), and/or a synthetic DNA vector (e.g., a synthetic circular DNA vector). Various elements can be included in such expression constructs, such as therapeutic genes, promoters, and regulatory elements that can enhance expression and/or persistence of the DNA vector in a target cell (such as a retinal cell as described herein). Nucleic acid vectors of the invention can include any of the expression constructs described herein, or combination thereof.

### A. Coding Sequences and Proteins

Some embodiments of expression constructs disclosed herein include one or more coding sequences for one or more genes, for example, ocular genes. In some embodiments, the ocular gene is a gene that is expressed in ocular tissue, such as, for example retinal tissue, which may include, for example, photoreceptor cells and/or retinal pigment epithelial (RPE) cells.

As non-limiting examples, genes that can be included in expression constructs disclosed herein can include one or more of the following. ABCA4, CEP290, CFH, ABCC6, RIMS1, LRP5, CC2D2A, TRPM1, IFT-172, C3, COL11A1, TUBGCP6, K1AA1549, CACNA1F, MYO7A, VCAN, USH2A, and HMCN1.

Coding sequences included in expression constructs can include genes that are involved in, for example, a retinal dystrophy, a Mendelian-heritable retinal dystrophy, and/or a disease selected from LCA, Stargardt Disease, pseudoxanthoma elasticum, rod cone dystrophy, exudative vitreoretinopathy. Joubert Syndrome, CSNB-1C, retinitis pigmentosa, age related macular degeneration (AMD), stickler syndrome, microcephaly and chorioretinopathy, retinitis pigmentosa, CSNB 2, Usher syndrome, and Wagner syndrome.

In some embodiments, the coding sequence in the expression construct is a human ABCA4 or MYO7A gene sequence. An exemplary human ABCA4 gene sequence is provided as National Center for Biotechnology Information (NCBI Reference Sequence: NG_009073. The amino acid sequence of an exemplary ABCA4 protein is given by Protein Accession No. P78363.3, and an ABCA4 amino acid sequence is provided here as SEQ ID NO: 32. An exemplary human MYO7A gene sequence is provided as NCBI Gene ID: 4647. The amino acid sequence of an exemplary MYO7A protein is given by Protein Accession No. Q13402, and a MYO7A amino acid sequence is provided here as SEQ ID NO: 31. In some embodiments, the coding sequence is a cDNA of ABCA4 or MYO7A. In some embodiments, the coding sequence is a codon optimized ABCA4 or MYO7A coding sequence. In some embodiments, the coding sequence encodes a functional variant of ABCA4, MYO7A, or another ocular gene. In some embodiments, the coding sequence of ABCA4 is or comprises SEQ ID NO: 1 or SEQ ID NO: 44. In some embodiments, the ABCA4 coding sequence comprises a nucleotide sequence having at least 75, 80, 85, 90, 95, 98, 99, 99.5 or 99.9% sequence identity or having 100% sequence identity to SEQ ID NO: 1 or SEQ ID NO: 44. In various embodiments, the ABCA4 coding sequence encodes the ABCA4 amino acid sequence of SEQ ID NO: 32, or a functional variant thereof (e.g., having from 1 to 10 or from 1 to 5 amino acid substitutions with respect to SEQ ID NO: 32). In some embodiments, the coding sequence of MYO7A is or comprises SEQ ID NO: 2 or SEQ ID NO: 45. In some embodiments, the MYO7A coding sequence comprises a nucleotide sequence having at least 75, 80, 85, 90, 95, 98, 99, 99.5 or 99.9% sequence identity or having 100% sequence identity to SEQ ID NO: 2 or SEQ ID NO: 45. In various embodiments, the MYO7A coding sequence encodes the amino acid sequence of SEQ ID NO: 31, or a functional variant (e.g., having from 1 to 10 or from 1 to 5 amino acid substitutions with respect to SEQ ID NO: 31). In various embodiments, the coding sequence, regulatory elements, promoters, enhancers, and other expression construct components may be human sequences.

In some embodiments, the coding sequences and regulatory sequences (collectively) included in the expression constructs and nucleic acid vectors described herein are greater than 4.5 Kb in length or greater than about 7 Kb in length. For example the coding sequences and regulatory sequences may be beyond the length that is suitable for viral vectors such as AA-V. For example, the coding sequences and regulatory sequences (collectively) are front 4.5 Kb to 25 Kb, or from 4.6 Kb to 24 Kb, or from 4.7 Kb to 23 Kb, or from 4.8 Kb to 22 Kb, or from 4.9 Kb to 21 Kb, or from 5.0 Kb to 20 Kb, or from 5.5 Kb to 18 Kb, or from 6.0 Kb to 17 Kb, or from 6.5 Kb to 16 Kb, or from 7.0 Kb to 15 Kb, or from 7.5 Kb to 14 Kb, or from 8.0 Kb to 13 Kb, or from 8.5 Kb to 12.5 Kb, or from 9.0 Kb to 12.0 Kb, or from 9.5 Kb to 11.5 Kb, or from 10.0 Kb to 11.0 Kb in length. In some embodiments, the coding and regulatory sequences are from 4.5 Kb to 8 Kb, or from 8 Kb to 10 Kb, or from 10 Kb to 15 Kb, or from 15 Kb to 20 Kb in length.

### B. Promoters

Expression constructs disclosed herein can comprise one or more promoters. In some embodiments, the promoter includes a native sequence derived from the endogenous promoter of an ocular gene. In some embodiments, the promoter includes a native sequence of the same gene to which it is operably linked. For example, an ABCA4 coding sequence can be operably linked to, and be under the control of, a sequence derived from the native ABCA4 genetic locus, such as a sequence upstream of the ABCA4 transcription start site. As another example, a MYO7A coding sequence can be operably linked to, and be under the control of, a sequence derived from the native MYO7A genetic locus, such as a sequence upstream of the MYO7A transcription start site. In some embodiments, the promoter sequence and coding sequence are derived from native sequences of the same species. For example, an expression construct may include an ABCA4 native promoter sequence from the haman genome and the ABCA4 coding sequence from the human genome or a functional variant thereof or a MYO7A native promoter sequence from the human genome and the MYO7A coding sequence from the human genome or a functional variant thereof.

In some embodiments, the expression construct comprises one or more of the following elements derived from native promoter sequences MYO7A Promoter HS1/2_Intron1 (SEQ ID NO: 3), MYO7A Promoter ISI-3 (SEQ ID NO: 4), MYO7A Promoter Min (SEQ ID NO: 5), ABCA4 Promoter Exon. Intron. Short (SEQ ID NO: 6), ABCA4 Promoter Exon_Intron1_large (SEQ ID NO: 7), or ABCA4 Promoter. Large (SEQ ID NO: 8), ABCA4 Promoter_Short (SEQ ID NO: 9), or functional variants thereof. In some embodiments, the expression construct comprises a nucleotide sequence having at least 75, 80, 85, 90, 95, 98, 99, 99.5 or 99.9% sequence identity or having 100% sequence identity, to any of SEQ ID NOs: 3-9. In some embodiments, the expression construct comprises a nucleotide sequence having at least 75, 80, 85, 90, 95, 98, 99, 99.5 or 99.9% sequence identity to a portion of any one of SEQ ID Nos: 3-9, such as least 100 contiguous nucleotides, or at least 200 contiguous nucleotides, or at least 300 contiguous nucleotides, or at least 500 contiguous nucleotides, or at least 750 contiguous nucleotides, or at least 1000 contiguous nucleotides, or at least 1500 contiguous nucleotides, or at least 2000 contiguous nucleotides or more of any of SEQ ID NOs: 3-9 For example, the expression construct may comprise a nucleotide sequence having at least 95% sequence identity to at least 500 or at least 1000 consecutive nucleotides of any one of SEQ ID Nos: 3-9.

In addition to the sequence sufficient to direct transcription, a promoter sequence can also include sequences of other regulatory elements that are involved in modulating transcription (e.g., enhancers, Kozak sequence). For example, in some embodiments, the expression construct comprises sequences derived from an ABCA4 or MYO7A native promoter, such as sequences derived from a native intron 1 sequence of ABCA4 or MYO7A. In some embodiments, regulatory elements, such as promoters, introns, insulators, enhancers, or other elements, are derived from native sequences of the same species as the gene to which they are operably linked in expression constructs.

In some embodiments, promoters included in the expression constructs are tissue-specific promoters in that, in normal operation, they drive expression only when present in certain tissue types, such as, for example, ocular tissue. In some embodiments, the promoter is not tissue-specific but is capable of driving expression in any tissue type. In some embodiments, the promoter is an inducible promoter. In some embodiments, the promoter is a constitutive promoter. In some embodiments, the construct comprises a cytomegalovirus (CMV) enhancer/beta-actin (CAG) promoter (e.g., SEQ ID NO: 14 or a functional variant thereof), elongation factor 1 alpha (EF1A) promoter (e.g., SEQ ID NO: 15 or a functional variant thereof), interphotoreceptor retinoid-binding protein (IBRP) promoter, rhodopsin kinase (RK) promoter (e.g., G protein-coupled receptor kinase 1 (GRK1) promoter), SV40 promoter, dihydrofolate reductase promoter, β-actin promoter, phosphoglycerol kinase (PGK) promoter, of functional variants thereof. Generally, functional variants of SEQ ID NO, 14 or 15 comprise a nucleotide sequence having at least 75, 80, 85, 90, 95, 98, 99, 99.5 or 99.9% sequence identity to SEQ ID NO: 14 or 15, or at or at least 200 contiguous nucleotides, or at least 300 contiguous nucleotides, or at least 500 contiguous nucleotides having such sequence identity.

### C. Other Regularly Elements

In addition to a promoter, the expression constructs can comprise other regulatory elements, which can provides appropriate transcription initiation, termination, and enhancer sequences; efficient RNA processing signals such as splicing and polyadenylation (polyA) signals; sequences that stabilize cytoplasmic mRNA; and sequences that enhance translation efficiency (i.e., Kozak consensus sequence).

For expression constructs that include genes encoding a protein, a polyadenylation (poly-A, or pA) sequence can be inserted following the gene (e.g., operably linked 3′ to the gene, e.g., directly linked 3′ to the gene).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Such 5′ non-transcribed regulatory sequences may include a promoter region that includes a promoter sequence for transcriptional control of the operably joined gene. Regulatory sequences may also include enhancer sequences or upstream activator sequences. The vectors of the disclosure may optionally include 5′ leader or signal sequences.

In some embodiments, the expression constructs include scaffold-matrix attachment regions (S/MARs). Without being bound by theory, it is believed that S/MAR elements can help establish long-term gene expression from a DNA vector through the interaction of the S/MAR element with the nuclear matrix. Known S/MAR constructs include the human IFN-γ S/MAR (SEQ ID NO: 10) and the human APOB S/MAR (NCBI Gene TD 106632268). Other known S/MAR elements can be included in the expression constructs. In some embodiments, a variant (SEQ ID NO: 11) of the IFN-γ S/MAR comprising tandem repeats of a functional portion of the IFN-γ S/MAR is included in the expression construct. In some embodiments, the expression construct comprises a nucleotide sequence having at least 75, 80, 85, 90, 95, 98, 99, 99.5 or 99.9% sequence identity or having 100% sequence identity to SEQ ID NO: 10 or 11. S/MAR elements can be operably linked either 5′ or 3′ to a coding sequence of an expression construct. In some embodiments, the S/MAR is operably linked 3′ to the coding sequence.

In some embodiments, the expression construct comprises chromatin insulator elements. In some embodiments, the one or more chromatin insulator elements may include one or more chicken hypersensitive site-4 elements (cHS4; SEQ ID NO: 12), which is a chromatin insulator from the chicken β-globin locus control region. In some embodiments, the expression construct includes a sequence having at least 75, 80, 85, 90, 95, 98, 99, 99.5 or 99.9% sequence identity or having 100% sequence identity to SEQ ID NO: 12.

In some embodiments, the expression construct comprises a regulatory element derived from (e.g., containing a portion of, or a variant thereof) a native sequence of ABCA4 intron 6. As described in the Examples provided herein, such regulatory elements, e.g., SEQ ID NO: 13, can enhance persistence and expression levels of genes operably linked thereto (including in ocular cells). Thus, some embodiments of the invention feature a regulatory element derived from a native sequence of ABCA4 intron 6, e.g., a sequence in the 5′ half of ABCA4 intron 6 (i.e., a sequence that is upstream from the midpoint between the 5′ and 3′ end of ABCA4 intron 6) or a sequence in the 5′ third of ABCA4 intron 6 (i.e., a sequence that is within the 5′-most 33.3% of ABCA4 intron 6).

In some instances, a regulatory element derived from ABCA4 intron 6 has one or more (one, two, three, four, five, or more) sequences that are identical to at least 100 consecutive nucleotides within a native human ABCA4 intron 6 (e.g. SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 29), e.g., from 100-5000, from 500 to 2500, from 1000 to 2000, or from 1500 to 1700 consecutive nucleotides within a native human ABCA4 intron 6 (e.g., SEQ ID NO: 29).

In some instances, a regulatory element derived from ABCA4 intron 6 has one or more (one, two, three, four, five, or more) sequences that are identical to at least 100 consecutive nucleotides within the 5′ half of a native human ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ half of a native human ABCA4 intron 6 (e.g., SEQ ID NO: 29), e.g., from 100-5000, from 500 to 2500, from 1000 to 2000, or from 1500 to 1700 consecutive nucleotides within the 5′ half of a native human ABCA4 intron 6 (e.g., SEQ ID NO: 29).

In some instances, a regulatory element derived from ABCA4 intron 6 has one or more (one, two, three, four, five, or more) sequences that are identical to at least 100 consecutive nucleotides within the 5′ third of a native human ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ third of a native human ABCA4 intron 6 (e.g., SEQ ID NO: 29), e.g., from 100-5000, from 500 to 2500, from 1000 to 2000, or from 1500 to 1700 consecutive nucleotides within the 5′ third of a native human ABCA4 intron 6 (e.g., SEQ ID NO: 29).

In some instances, sequences shared between the regulatory element and ABCA4 intron 6 include nucleotides wholly or partially within nucleotides 3158-4822 of ABCA4 (numbering starting from the 5′ end of ABCA4 intron 6), e.g., SEQ ID NO: 13.

In some instances, a regulatory element is a functional variant of any of the aforementioned ABCA4 intron 6-derived regulatory elements. For example, in some instances, a regulatory element derived from ABCA4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides within a native human ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 29), e.g., from 100-5000, from 500 to 2500, from 1000 to 2000, or from 1500 to 1700 consecutive nucleotides within a native human ABCA4 intron 6 (e.g., SEQ ID NO: 29). In some instances, sequences shared or with the level of identity between the regulatory element and ABCA4 intron 6 include nucleotides wholly or partially within nucleotides 3158-4822 of ABCA4 (numbering starting from the 5′ end of ABCA4 intron 6), e.g., SEQ ID NO: 13.

In some instances, a regulatory element derived from ABCA4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides within the 5′ half of a native human ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ half of a native human ABCA4 intron 6 (e.g., SEQ ID NO: 29), e.g., from 100-5000, from 500 to 2500, from 1000 to 2000, or from 1500 to 1700 consecutive nucleotides within the 5′ half of a native human ABCA4 intron 6 (e.g., SEQ ID NO: 29). In some instances, sequences shared or with the level of identity between the regulatory element and ABCA4 intron 6 include nucleotides wholly or partially within nucleotides 3158-4822 of ABCA4 (numbering starting from the 5′ end of ABCA4 intron 6), e.g., SEQ ID NO: 13.

In some instances, a regulatory element derived from ABCA4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides within the 5′ third of a native human ABCA4 intron 6 (e.g., SEQ ID NO: 29), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ third of a native human ABCA4 intron 6 (e.g., SEQ ID NO: 29), e.g., from 100-5000, from 500 to 2500, from 1000 to 2000, or from 1500 to 1700 consecutive nucleotides within the 5′ third of a native human ABCA4 intron 6 (e.g., SEQ ID NO: 29). In some instances, sequences shared or with the level of homology between the regulatory element and ABCA4 intron 6 include nucleotides wholly or partially within nucleotides 3158-4822 of ABCA4 intron 6 (numbering starting from the 5′ end of ABCA4 intron 6), e.g., SEQ ID NO: 13.

In some instances, a regulatory element derived from ABCA4 intron 6 has one or more (one, two, three, four, five, or more) sequences that are identical to at least 100 consecutive nucleotides of nucleotides 31584822 of ABCA4 intron 6, e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides of nucleotides 3158-4822 of ABCA4 intron 6).

In some instances, a regulatory element derived from ABC A4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides of nucleotides 3158-4822 of ABCA4 intron 6, e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides of nucleotides 3158-4822 of ABCA4 intron 6).

In some embodiments, the expression construct includes a sequence having at least 75, 80, 85, 90, 95, 98, 99, 99.5 or 99.9% sequence identity or having 100% sequence identity to SEQ ID NO: 13.

in some embodiments, the expression construct includes a sequence having at least 75, 80, 85, 90, 95, 98, 99, 99.5 or 99.9% sequence identity or having 100% sequence identity to nucleotides 3158 to 4822 of ABCA4 intron 6.

in some of any of the aforementioned instances, the regulatory element derived from ABCA4 intron 6 has been mutated in one or more positions (e.g., one, two, three, or more positions), relative to the native ABCA4 intron 6 sequence, to remove a recognition site of a restriction enzyme, e.g., a type IIs restriction enzyme (e.g., BsaI), which can improve manufacturing efficiency by streamlining cell-free production of synthetic circular DNA vectors (e.g., by consolidating steps by using a type IIs restriction enzyme). For instance, nucleotide 3530 of native human ABCA4 intron 6 (SEQ ID NO: 29), which is a G, can be deleted to remove a BsaI recognition site in a regulatory element derived from ABCA4 intron 6, thereby facilitating an improved, BsaI-based manufacturing process. For example, in some embodiments, a nucleotide sequence from nucleotides 3,158 to 4,822 of native ABCA4 intron 6 is modified to delete of G3530, thereby producing the ABCA4 intron 6-derived regulatory element of SEQ ID NO: 13.

Promoters, coding sequences, and other elements can be included in the expression constructs in any suitable order that provides for effective expression and/or persistence. In some embodiments, an expression construct includes, in a 5′ to 3′ direction, a promoter, a coding sequence, and a polyadenylation sequence. In some embodiments, an expression construct includes, in a 5′ to 3′ direction, an enhancer sequence (e.g., an S/MAR), a promoter, a coding sequence, and a polyadenylation sequence. In some embodiments, an expression construct includes, in a 5′ to 3′ direction, an insulator sequence (e.g., cHS4), a promoter, a coding sequence, and a polyadenylation sequence. In some embodiments, an expression construct includes, in a 5′ to 3′ direction, an insulator sequence (e.g., cHS4), a promoter, a coding sequence, a polyadenylation sequence, and another regulatory element (e.g., a regulatory element derived from intron 6 of ABCA4). In some embodiments, an expression construct includes, in a S′ to 3′ direction, an insulator sequence (e.g., cHS4), a promoter, a coding sequence, a polyadenylation sequence, and an enhancer sequence (e.g., an S/MAR). In some embodiments, an expression construct includes, in a 5′ to 3′ direction, an insulator sequence (e.g., cHS4), a promoter, a coding sequence, another regulatory element (e.g., a regulatory element derived from intron 6 of ABCA4), and a polyadenylation sequence. In some embodiments, an expression construct includes, in a 5′ to 3′ direction, an insulator sequence (e.g., cHS4), a promoter, a coding sequence, an enhancer sequence (e.g., an S/MAR), and a polyadenylation sequence. In some embodiments, an expression construct includes, in a 5′ to 3′ direction, a promoter, a regulatory element (e.g., an intron), a coding sequence, and a polyadenylation sequence. In some embodiments, an expression construct includes, in a 5′ to 3′ direction, an enhancer sequence (e.g., an S/MAR), a promoter, a regulatory element (e.g., an intron or portion thereof, e.g., a portion of ABCA4 intron 6, e.g., an intron 6 regulatory element), a coding sequence, and a polyadenylation sequence. In some embodiments, an expression construct includes, in a 5′ to 3′ direction, an insulator sequence (e.g., cHS4), a promoter, a regulatory element (e.g., an intron or portion thereof, e.g., a portion of ABCA4 intron 6, e.g., an intron 6 regulatory element), a coding sequence, and a polyadenylation sequence. In some embodiments, an expression construct includes, in a 5′ to 3′ direction, an insulator sequence (e.g., cHS4), a promoter, a regulatory element (e.g., an intron or portion thereof, e.g., a portion of ABCA4 intron 6, e.g., an intron 6 regulatory element), a coding sequence, a polyadenylation sequence, and another regulatory element (e.g., a regulatory element derived from intron 6 of ABCA4). In some embodiments, an expression construct includes, in a S′ to 3′ direction, an insulator sequence (e.g., cHS4), a promoter, a regulatory element (e.g., an intron or portion thereof, e.g., a portion of ABCA4 intron 6, e.g., an intron 6 regulatory element), a coding sequence, a polyadenylation sequence, and an enhancer sequence (e.g., an S/MAR). In some embodiments, an expression construct includes, in a 5′ to 3′ direction, an insulator sequence (e.g., cHS4), a promoter, a regulatory element (e.g., an intron or portion thereof, e.g., a portion of ABCA4 intron 6, e.g., an intron 6 regulatory element), a coding sequence, another regulatory element (e.g., a regulatory element derived from intron 6 of ABCA4), and a polyadenylation sequence. In some embodiments, an expression construct includes, in a 5′ to 3′ direction, an insulator sequence (e.g., cHS4), a promoter, a regulatory element (e.g., an intron or portion thereof, e.g., a portion of ABCA4 intron 6, e.g., an intron 6 regulatory element), a coding sequence, an enhancer sequence (e.g., an S/MAR), and a polyadenylation sequence. Sequence elements disclosed herein can be arranged in other suitable combinations and orders.

Exemplary nucleic acid vector constructs are provided as SEQ ID NO: 27 and SEQ ID NO: 30, which each comprise (in 5′ to 3′ order) (i) a CAG promoter (SEQ ID NO: 14) (which may alternatively be a functional derivative of a CAG promoter described herein), (ii) an ABCA4 coding sequence (SEQ ID NO: 44) (which may alternatively be a functional derivative of such coding sequence as described herein); and (iii) an S/MAR regulatory sequence (SEQ ID NO: 10 in the case of SEQ ID NO: 27, SEQ ID NO: 11 in the case of SEQ ID NO: 30)(and which may be a functional derivative of such sequence as described herein). Functional derivatives may have, for example, at least 75, 80, 85, 90, 95, 98, 99, 99.5 or 99.9% sequence identity to the reference sequence, while retaining the expression level or persistence thereof in ocular cells.

In some embodiments, such constructs further comprise an the insulator sequence at the 5′ side of the CAG promoter, which may be a cHS4 insulator, such as that comprising SEQ ID NO: 12 or a functional derivative thereof (as described herein).

### III. Nucleic Acid Vectors

Provided herein are nucleic acid vectors that include any of the expression constructs described herein, or components (e.g., regulatory elements) or combinations thereof. The nucleic acid vectors can be produced according to known methods as plasmid DNA vectors, nanoplasmid vectors (as described in, e.g., WO 2008/153733 and WO 2014/035457), minicircle DNA vectors (as described in, e.g., U.S. Pat. Nos. 8,828,726 and 9,233,174), mini-intronic plasmids (described in, e.g., Lu et al., Mol. Ther. 2013, 21:954 and U.S. Pat. No. 9,347,073), synthetic circular DNA vectors as described herein and in WO 2019/178500, closed-ended DNA vectors (as described, e.g., in U.S. 2020/0283794 and 2021/007119?), doggybone DNA vectors (as described, e.g., in U.S. 2015/0329902 and U.S. Pat. No. 9,499,847), or ministring DNA vectors (as described, e.g., in U.S. Pat. Nos. 9,290,778 and USRE48908E1). In particular embodiments, any of the nucleic acid vectors described herein comprise a therapeutic sequence (e.g., an ocular gene-encoding sequence, such as ABCA4 or MYO7A). In some instances, a nucleic acid vector of the invention is a nonviral DNA vector (e.g., a naked DNA vector), a circular DNA vector (e.g., a supercoiled circular DNA vector), and/or a synthetic DNA vector (e.g., a synthetic circular DNA vector), which comprises. (i) a cDNA sequence encoding ABCA4 and a regulatory element comprising a sequence derived from ABCA4 intron 6; or (ii) a native ABCA4 promoter or functional variant thereof, a sequence encoding ABCA4, and a regulatory element (e.g., a regulatory element comprising a scaffold/matrix attachment region (S/MAR) sequence or a sequence derived from ABCA4 intron 6), or (iii) a regulatory element comprising an insulator sequence (e.g., cHS4) and an ocular gene-encoding sequence; or (iv) a promoter comprising any one of SEQ ID NOs: 3-5 and a sequence encoding MYO7A; or (v) a therapeutic protein-encoding sequence (e.g., an ocular gene-encoding sequence) and a scaffold/matrix attachment region (S/MAR) sequence; or (vi) a promoter comprising the nucleotide sequence of any one of SEQ ID NOs: 6-9 or a functional variant thereof and a sequence encoding ABCA4.

In some instances, the nucleic acid vectors are circular DNA vectors that persist intracellularly (e.g., in dividing or in quiescent cells, such as post-mitotic cells) as episomes, e.g., in a manner similar to AAV vectors. In any of the embodiments described herein, a circular DNA vector may be a non-integrating vector. Circular DNA vectors provided herein can be naked DNA vectors, devoid of components inherent to viral vectors (e.g., viral proteins) and bacterial plasmid DNA, such as immunogenic components (e.g., immunogenic bacterial signatures, such as CpG islands or CpG motifs) or components additionally, or otherwise associated with reduced persistence (e.g., CpG islands or CpG motifs). Circular DNA vectors feature one or more therapeutic sequences and may lack plasmid backbone elements, such as (i) a bacterial origin of replication and/or (ii) a drug resistance gene and/or (iii) a recombination site. Synthetic circular DNA vectors lacking an origin or replication can be synthesized through various means known in the art and described herein. Synthesis methods may involve use of phage polymerase, such as Phi29 polymerase, as a replication tool using, e.g., rolling circle amplification. Particular methods of cell-free synthesis of synthetic circular DNA vectors are further described in International Patent Publication No. WO 20191178500, which is hereby incorporated by reference.

In other embodiments, therapeutic circular DNA vectors described herein can be non-synthetic vectors (e.g., containing bacterial backbone sequences such as an origin of replication and/or recombination site). Such nucleic acid vector can be in vivo-produced, and may lack a selectable marker (e.g., drug resistance gene) and optionally a recombination site, e.g., by using engineered bacterial cells to produce circular DNA vectors from a parental plasmid. Bacterial cells (e.g., E. coli) can be engineered to contain a Rep gene encoding a bacterial replication protein, which is optionally integrated into the bacterial genome. The engineered cells can be transfected with a parental plasmid having a vector sequence and a backbone sequence. The vector sequence includes an on sequence corresponding to the Rep gene and does not include a selectable marker. The backbone sequence includes a selectable marker and does not include the on sequence included in the vector sequence. The parental plasmid may also have restriction enzyme recognition sequences, or site-specific recombination sequences, or transposase recognition sequences flanking the vector sequence arranged so that the plasmid backbone sequence can be separated from the vector sequence inside the cell by restriction digestion, site-specific recombination, or transposase action. In the case of restriction digestion, the circular DNA vector is then formed by self-ligation of the vector sequence. In the case of site-specific recombination or transposase action, the circular DNA vector is formed as recombination or transposase action is completed. Expression of the rep protein after separation of the vector sequence and formation of the circular DNA vector can maintain the circular DNA vector at a high copy number, despite the circular DNA vector lacking a selectable marker. In contrast, maintenance of the plasmid backbone sequence in the engineered bacterial cell after separation can be avoided by changing the culture conditions to remove selective pressure for the selectable marker. Culturing of a population of bacterial cells with a high copy number of circular DNA vector under conditions in which the parental plasmid is not maintained can efficiently produce a high yield of highly pure circular DNA vector. Such methods are described in WO 2023/122625 and 63/509,458 (filed Jun. 21, 2023), which are hereby incorporated by reference in their entireties.

One benefit of using a transposase-based system is the ability to further reduce the backbone size within the circular DNA vector. For instance, use of a site-specific recombinase results in a recombination site (e.g., an attachment site) within the vector, near or adjacent to the replication origin. In contrast, use of a transposase allows the replication origin to directly connect the 5′ end of the therapeutic sequence to 3 end of the therapeutic sequence without intervening sequences. In some instances, use of a transposase allows for a “scarless” backbone by positioning the resulting sequence of the transposition (the transposase overhang) within the therapeutic sequence without modifying the function of the therapeutic sequence. As an example, piggybac transposase produces a four-bp transposase overhang of TTAA. By positioning the plasmid backbone within the sequence of interest at a TTAA site, one can design the system such that, upon transposase-mediated excision of the plasmid backbone from the sequence of interest, the original sequence of interest is restored, leaving only the original TTAA sequence as the transposase scar. This leaves the backbone within the circular DNA vector free of a transposase scar. Thus, the plasmid backbone sequences in the vector can consist entirely of replication origin.

Additionally, or alternatively, the transposase scar may be positioned within the vector backbone (e.g., within the sequence containing the replication origin). For instance, if the parental plasmid contains inverted repeats (left-end) and (right-end) flanking the backbone, and or transposase overhang sequences flanking the therapeutic sequence, the transposase scar will be positioned between the 3′ and 5′ ends of the sequence of interest (e.g., next to the origin of replication).

In some embodiments, the engineered bacterial cells for producing the circular DNA vector of this disclosure include a Rep gene encoding a bacterial replication protein directing replication from ColE2-P9 origin, and which may be integrated into the bacterial genome. Alternatively, the Rep gene is included on an extrachromosomal DNA molecule such as, for example, a plasmid or a bacterial artificial chromosome (“BAC”). The engineered bacterial cells further comprise a parental plasmid comprising a vector sequence and a backbone sequence. The vector sequence includes a replication origin (ori) sequence corresponding to the Rep gene and does not include a selectable marker. The backbone sequence includes a gene encoding a selectable marker and does not include the on sequence included in the vector sequence. The parental plasmid also has enzyme recognition sequences (e.g., restriction enzyme recognition sequences, site-specific recombination sequences, or transposase recognition sequences) flanking the vector sequence arranged so that the plasmid backbone sequence can be separated from the vector sequence inside the cell by restriction digestion, transposition, or site-specific recombination.

In some embodiments, a short origin of replication is used in the circular DNA vector to minimize bacterial sequences, such as a ColE2-P9 replication origin, or a functional variant thereof. In such embodiments, the Rep gene encodes a ColE2-P9 replication protein. In some exemplary embodiments, the Rep gene encodes a ColE2-P9 replication protein that has the amino acid sequence set forth in SEQ ID NO: 33 (or a functional variant thereof, for example, having at least 80%, or at least 90%, or at least 95%, or at least 97%, or at least 98%, or at least 99% sequence identity thereto). Other suitable replication proteins include replication proteins encoded by naturally-occurring plasmids, including, for example, those that are related to ColE2-P9, such as ColE3-CA38.

In some exemplary embodiments, the on (e.g., one strand) comprises or consists of a nucleotide sequence as set forth in SEQ ID NO: 34. In some embodiments, the on sequence is a functional fragment of the ColE2-P9 ort sequence that has the DNA sequence (on one strand) set forth in SEQ ID NO: 34. The 40 base pair functional fragment set forth in SEQ ID NO: 34 is capable of supporting vector replication in a cell expressing the ColE2-P9 replication protein. In some embodiments, the ori is ColE2-P9 origin and is no more than about 40 nucleotides in length, or no more than 38 nucleotides in length, no more than 37 nucleotides in length, or no more than 36 nucleotides in length, or no more than 34 nucleotides in length, or no more than 30 nucleotides in length. In various embodiments, the ColE2-P9 origin is from 20 to 40 nucleotides in length, or from 30 to 40 nucleotides in length, or from 34 to 40 nucleotides in length, thereby minimizing bacterial-derived sequences in the circular vector In some embodiments, the ori sequence is a naturally occurring on sequence.

In some instances, the or sequence is a functional variant of a naturally occurring ori, such as, for example, an oni sequence that has been modified to be shorter than a corresponding naturally occurring on sequence, while still retaining the ability to support replication initiation. Such functional variants of the ColE2-P9 replication origin include SEQ ID NOs 35-43 Such sequences are shown herein as a single strand for convenience, although it is recognized that the origin will be present in the vector as double-stranded DNA. In some embodiments, the functional variant has 1, 2, 3, 4, or 5 nucleotide substitutions with respect to a origin sequence of SEQ ID NOS: 35-43. With respect to SEQ ID NO: 43, each X is selected from A, T, C, or G. In some embodiments: X1 is A, T, or C; X2 is A, T, or C; X3 is A, T, or G; X4 is A, T, or C, X5 is A, T, or G; X6 is C; X7 is A.

In some instances, circular DNA vectors are naked DNA vectors and are substantially devoid of components inherent to viral vectors (e.g., viral proteins) and bacterial plasmid DNA, such as immunogenic components (e.g., immunogenic bacterial signatures (e.g., CpG motifs)) or components additionally or otherwise associated with reduced persistence (e.g., CpG islands). In some embodiments, the circular DNA vector contains DNA in which at least 50% (e.g., at least 60%, at least 70%, at least 80%, at least 90%, at least 95%, at least 97%, at least 99%, or essentially all) of the DNA lacks one or more elements of bacterial plasmid DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 99%, or essentially all) of the DNA lacks CpG methylation. In some embodiments, the circular DNA vector contains DNA in which at least 50% (e.g., at least 60%, at least 70%, at least 80%, at least 90%, at least 95%, at least 97%, at least 99%, or essentially all) of the DNA lacks bacterial methylation signatures, such as Dam methylation and Dcm methylation. For example, in some embodiments, the circular DNA vector contains DNA in which at least 50% (e.g., at least 60%, at least 70%, at least 80%, at least 90%, at least 95%, at least 97%, at least 99%, or essentially all) of the GATC sequences are unmethylated (e.g., by Dam methylase). Additionally, or alternatively, the circular DNA vector contains DNA in which at least 50% (e.g., at least 60%, at least 70%, at least 80%, at least 90%, at least 95%, at least 97%, at least 99%, or essentially all) of the CCAGG sequences and/or CCTGG sequences are unmethylated (e.g., by Dcm methylase).

In some embodiments, the circular DNA vector is persistent in vivo (e.g., the therapeutic circular DNA vector exhibits improved expression persistence (e.g., intra-cellular persistence and/or trans-generational persistence) and/or therapeutic persistence relative to a reference vector, e.g., a circular DNA vector having one or more bacterial signatures not present in the vector of the disclosure).

In some embodiments, expression of a circular DNA vector persists for at least two weeks, at least three weeks, at least four weeks, at least six weeks, at least two months, at least three months, at least four months, at least five months, at least six months, at least seven months, at least eight months, at least nine months, at least ten months, at least eleven months, one year, or longer after administration. In some embodiments, the circular DNA vector persists for at least about six months or at least one year, or at least 18 months, or at least two years in ocular cells (such as photoreceptor cells and/or RPE cells). In some embodiments, the expression level of the circular DNA vector does not decrease by more than 90%, or by more than 50%, or by more than 25%, or by more than 10% in the 1 week or more, e.g., 2 weeks, 3 weeks, 5 weeks, 7 weeks, 9 weeks or more, 13 weeks or more, or 18 weeks or more following transfection from levels observed within the first 1, 2, or 3 days. In some embodiments, administration of the nucleic acid vector of this disclosure (e.g., to retinal cells) is no more than 4 times per year, or no more than 2 times per year, or no more than once per year, or even less frequently (e.g., every two years).

In embodiments, the circular DNA vector is monomeric. In some embodiments, the circular DNA vector is supercoiled, e.g., following treatment with a topoisomerase (e.g., gyrase).

In some embodiments, the therapeutic protein is an antibody, or a functional variant thereof. Antibodies include fragments that are capable of binding to an antigen, such as Fv, single-chain Fv (scFv), Fab, Fab′, di-scFv, sdAb (single domain antibody), (Fab′)2 (including a chemically linked F(ab′)2), and nanobodies.

In embodiments of circular DNA vectors involving a non-protein coding therapeutic sequence, the therapeutic sequence lacks a protein-coding domain (e.g., a therapeutic protein-coding domain). For instance, in some embodiments, a therapeutic sequence includes a non-protein-coding therapeutic nucleic acid, such as a short hairpin RNA (shRNA)-encoding sequence or an immune activating therapeutic nucleic acid (e.g., a TLR agonist).

In some embodiments, the therapeutic sequence includes a reporter sequence in addition to a therapeutic protein-encoding domain or a therapeutic non-protein encoding domain. Such reporter genes can be useful in verifying therapeutic gene sequence expression, for example, in specific cells and tissues. Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the an. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. In some embodiments, the therapeutic sequence lacks a reporter sequence.

In some instances, any of the nucleic acid vectors of the disclosure may encode a self-replicating RNA molecule. Such self-replicating RNA molecules include replicase sequences derived from alphavirus, which are characterized as having positive-stranded replicons that are translated after delivery to a target cell into a replicase for replicase-transcriptase). The replicase is translated as a polyprotein which auto-cleaves to provide a replication complex which creates genomic negative-strand copies of the positive-strand delivered RNA. These negative-strand transcripts can themselves be transcribed to give further copies of the positive-stranded parent RNA and also to give a subgenomic transcript (e.g., a modulatory sequence). Translation of the subgenomic transcript thus leads to in situ expression of the modulatory protein by the infected cell.

Non-limiting examples of alphaviruses from which replicase-encoding sequences of the present invention can be derived include Venezuelan equine encephalitis virus (VEE), Semliki Forest virus (SF), Sindbis virus (SIN), Eastern Equine Encephalitis virus (EEE), Western equine encephalitis virus (WEE), Everglades virus (EVE), Mucambo virus (MUC), Pixuna virus (PIX), Semliki Forest virus (SF), Middelburg virus (MID), Chikungunya virus (CHIK), U'Nyong-Nyong virus (ONN). Ross River virus (RR), Barmah Forest virus (BF), Getah virus (GET), Sagiyama virus (SAG), Bebaru virus (BEB), Mayaro virus (MAY), Una virus (UNA), Aura virus (AURA), Bahanki virus (BAB), Highlands J virus (HJ), and Fort Morgan virus (FM). In particular instances of the invention, the self-replicating RNA molecule comprises a VEE replicase or a variant thereof.

Mutant or wild-type virus sequences can be used. For example, in some instances, the self-replicating RNA includes an attenuated TC83 mutant of VEE replicase. Other mutations in the replicase are contemplated herein, including replicase mutated replicases (e.g., mutated VEE replicases) obtained by in vitro evolution methods, e.g., as taught by Yingzhong et al., Sci Rep. 2019, 9. 6932, the methodology of which is incorporated herein by reference.

In some instances, a self-replicating RNA molecule includes (i) a replicase-encoding sequence (e.g., an RNA sequence that encodes an RNA-dependent RNA polymerase which can transcribe RNA from the self-replicating RNA molecule) and (ii) an ocular gene. The polymerase can be an alphavirus replicase, e.g., an alphavirus replicase comprising one, two, three, or all four alphavirus nonstructural proteins nsP1, nsP2, nsP3, and nsP4. In some instances, the polymerase is a VEE replicase, e.g., a VEE replicase comprising one, two, three, or all four alphavirus nonstructural proteins nsP1, nsP2, nsP3, and nsP4.

In other embodiments, the nucleic vector is a therapeutic nucleic vector that does not encode a reporter molecule, such as green fluorescent protein (GFP).

Expression constructs described herein if desired can be assembled into viral vectors, such as vectors consisting of, or derived from, adeno-associated virus (AAV), adenovirus, Retroviridae family virus, parvovirus, coronavirus, rhabdovirus, paramyxovirus, picornavirus, alphavirus, herpes virus, or poxvirus.

In some instances, the nucleic acid vector is a non-viral DNA vector (e.g., the DNA vector is not encapsulated within a viral capsid). Additionally, or alternatively, in some embodiments, the nucleic acid vector is not encapsulated in an envelope (e.g., a lipid envelope) or a matrix (e.g., a polymer matrix) and is not physically associated with (e.g., covalently or non-covalently bound to) a solid structure (e.g., a particulate structure) prior to and upon administration to the individual in some embodiments, the nucleic acid vector is untethered to any adjacent nucleic acid vectors such that, in a solution of nucleic acid vectors, each nucleic acid vector is free to diffuse independently of adjacent nucleic acid vectors. In some embodiments, the nucleic acid vector is associated with another agent in liquid solution, such as a charge-altering molecule or a stabilizing molecule.

The nucleic acid vector may be a naked DNA vector, i.e., not complexed with another agent (e.g., encapsulated within, conjugated to, or non-covalently bound to another agent). Naked DNA vectors may be co-formulated (e.g., in solution) with agents that are not complexed with the naked DNA vector, such as buffering agents and/or agents that are generally recognized as safe (GRAS) by the U.S. Food and Drug Administration.

### IV. Pharmaceutical Compositions

In some embodiments, a pharmaceutical composition of the disclosure contains at least about 0.1 mg of DNA vector, or at least about 0.5 mg, or at least about 1.0 mg DNA vector or at least about 2.0 mg of DNA vector, or at least 5 mg of DNA vector, or at least 10 mg of DNA vector in a pharmaceutically acceptable carrier. In some embodiments, the pharmaceutical composition of the invention is substantially devoid of impurities. For instance, in some embodiments, the pharmaceutical formulation contains <2.0% protein content by mass (e.g., <1.5%, <1.0%, <0.5%, <0.1%, <0.05%, or <0.01% protein content by mass). In some instances, protein content is determined by bicinchoninic acid assay. Additionally or alternatively, protein content is determined by ELISA.

In some instances, the pharmaceutical composition contains <5.0% RNA content by mass (e.g., <4.0%, <3.0%, <2.0%, <1.5%, <0.5%, <0.1%, <0.05%, or <0.01% RNA content by mass). In some embodiments, the RNA content is determined by agarose gel electrophoresis. In some embodiments, the RNA content is determined by quantitative PCR. In some embodiments, the RNA content is determined by fluorescence assay (e.g., Ribogreen).

In some embodiments, the pharmaceutical composition contains <5.0% gDNA content by mass (e.g., =4.0%, <3.0%, <2.0%, <1.5%, <1.0%, <0.5%, <0.1%, <0.05%, or <0.01% gDNA content by mass). In some embodiments, the gDNA content is determined by agarose gel electrophoresis or capillary electrophoresis. In some embodiments, the gDNA content is determined by quantitative PCR. In some embodiments, the gDNA content is determined by Southern blot.

In some embodiments, the pharmaceutical composition contains <40 EU/mg endotoxin. In some embodiments, the pharmaceutical formulation contains <20 EU/mg endotoxin. In some embodiments, the pharmaceutical formulation contains <10 EU/mg endotoxin. In some embodiments, the pharmaceutical formulation contains <5 EU/mg endotoxin (e.g., <4 EU/mg endotoxin, <3 EU/mg endotoxin, <2 EU/mg endotoxin, <1 EU/mg endotoxin, <0.5 EU/mg endotoxin), e.g., as measured by Limulus Amoebocyte Lysate (LAL) assay.

In some embodiments, pharmaceutical compositions comply with current good manufacturing practice (GMP) according to the standards promulgated by the U.S. Food & Drug Administration and set forth in 21 C.F.R. Parts 210 and 211, which are incorporated herein by reference in their entirety.

Pharmaceutical compositions provided herein may include one or more pharmaceutically acceptable carriers, such as excipients and/or stabilizers that are nontoxic to the individual being treated (e.g., human patient) at the dosages and concentrations employed. In some embodiments, the pharmaceutically acceptable carrier is an aqueous pH buffered solution. Examples of pharmaceutically acceptable carriers include buffers such as phosphate, citrate, and other organic acids, antioxidants including ascorbic acid; low molecular weight (less than about 10 residues) polypeptide; proteins, such as serum albumin, gelatin, or immunoglobulins; hydrophilic polymers such as polyvinylpyrrolidone, amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

If the pharmaceutical composition is provided in liquid form, the pharmaceutically acceptable carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na2CO2, NaHCO2, and Na2SO4. Examples of potassium salts include, e.g., KCl, KI, KBr, K2CO2, KHCO2, and K2SO4. Examples of calcium salts include, e.g., CaCl2, CaI2, CaBr2, CaCO2, CaSO4, and Ca(OH)2.

Additionally, organic anions of the aforementioned cations may be contained in the buffer. According to a particular embodiment, the buffer suitable for injection purposes as defined above, may contain salts selected from sodium chloride (NaCl), calcium chloride (CaCl2) or potassium chloride (KCl), wherein further anions may be present. CaCl2 can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl2). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt, gelatin; tallow, solid glidants, such as stearic acid, magnesium stearate; calcium sulfate; vegetable oils, such as groundnut oil, cottonseed oil, sesame oil, olive oil, corn oil; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers, antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution.

In certain embodiments of the invention, any of the DNA vectors can be complexed with one or more cationic or polycationic compounds, e.g., cationic or polycationic polymers, cationic or polycationic peptides or proteins, e.g. protamine, cationic or polycationic polysaccharides, and/or cationic or polycationic lipids.

According to a particular embodiment, the DNA vector may be complexed with lipids to form one or more liposomes, lipoplexes, or lipid nanoparticles. Therefore, in one embodiment, the pharmaceutical composition comprises liposomes, lipoplexes, and/or lipid nanoparticles comprising a DNA vector.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for circular DNA vectors. Cationic lipids, such as MAP. (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Thus, in one embodiment of the invention, a circular DNA vector is complexed with cationic lipids and/or neutral lipids and thereby forms liposomes, lipid nanoparticles, lipoplexes or neutral lipid-based nanoliposomes in the present pharmaceutical compositions.

In a particular embodiment, a pharmaceutical composition comprises the circular DNA vector of the invention that is formulated together with a cationic or polycationic compound and/or with a polymeric carrier. Accordingly, in a further embodiment of the invention, the circular DNA vector as defined herein is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4.1 (w/w) to about 1:1 (w/w) or of about 3:1 (w/w) to about 1.1 (w/w), e.g., from about 3.1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the circular DNA vector to the one or more polycations is in the range of about 0.1 to 10, including a range of about 0.3 to 4, of about 0.5 to 2, of about 0.7 to 2 and of about 0.7 to 1.5.

The nucleic acid vectors described herein can also be associated with a vehicle, transfection or complexation agent for increasing the transfection efficiency and/or the expression of the modulatory gene according to the invention.

In some instances, the circular DNA vector according to the invention is complexed with one or more polycations, preferably with protamine or oligofectamine. Further cationic or polycationic compounds, which can be used as transfection or complexation agent may include cationic polysaccharides, for example chitosan, polybrene, cationic polymers, e.g. polyethyleneimine (PEM), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio)propane, DC-6-14: O,O-ditetradecanoyl-N-(α-trimethylammonioacetyl)diethanolaminechloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAFE), (e.g., diamine end modified 1,4 butanediol diacrylate-co-5-amino-1-pentanol polymers, or polymers described in U.S. Pat. No. 8,557,231, PEGylated PBAE, such as those described in U.S. Patent Application No. 2018/0112038; any suitable polymer disclosed in Green et al., Acc. Chem. Res. 2008, 41(6): 749-759, such as diamine end modified 1,4 butanediol diacrylate-co-5-amino-1-pentanol polymers; any suitable modified PBAE as described in International Patent Publication No. WO 2020/077159 or WO 2019/070727; PBAE 457 as disclosed in Shen et al., Sci. Adv 2020, 6: eaba1606, etc.), dendrimers, such as polypropylamine dendrimers or pAMAM based dendrimers, etc., polyimine(s), such as PET poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g., selected from a cationic polymer as mentioned above) and of one or more hydrophilic or hydrophobic blocks (e.g. polyethyleneglycole); etc.

According to a particular embodiment, the pharmaceutical composition includes the DNA vector encapsulated within or attached to a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herein by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector The cationic or polycationic peptide, protein or polymer, may be a linear molecule; however, branched cationic or polycationic peptides, proteins or polymers may also be used.

In some embodiments, any of the DNA vectors described herein can be complexed with, or encapsulated using, any of the components, peptides, or particles taught PCT/US2023/065763, which is hereby incorporated by reference.

Every disulfide-crosslinking cationic or polycationic protein, peptide or polymer of the polymeric carrier, which may be used to complex the DNA vector according to the invention included as part of the pharmaceutical composition may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the DNA vector of the present invention may be formed by disulfide-crosslinked cationic (or polycationic) components. In particular, such cationic or polycationic peptides or proteins or polymers of the polymeric carrier, which comprise or are additionally modified to comprise at least one SH moiety, can be selected from proteins, peptides, and polymers as a complexation agent.

In other embodiments, the DNA vector according to the disclosure may be administered naked in a suitable buffer without being associated with any further vehicle, transfection, or complexation agent.

### V. Methods of Use

### A. Methods of Expression and Treatment

In certain aspects, the disclosure provides methods of inducing expression (e.g., persistent expression) of a therapeutic sequence or transgene in a subject in need thereof (e.g., as part of a gene therapy regimen) by administering to the subject any of the nucleic acid vectors described herein, or pharmaceutical compositions thereof: Thus, the present methods include administering to a subject (i) a nucleic acid vector (or pharmaceutical composition thereof) including a cDNA sequence encoding ABCA4 and a regulatory element comprising a sequence derived from ABCA4 intron 6, or (ii) a nucleic acid vector (or pharmaceutical composition thereof) comprising a native ABCA4 promoter or functional variant thereof, a sequence encoding ABCA4, and a regulatory element (e.g., a regulatory element comprising a scaffold/matrix attachment region (S/MAR) sequence, or functional variant thereof, or a sequence derived from ABCA4 intron 6) or (iii) a nucleic acid vector (or pharmaceutical composition thereof) comprising a first regulatory element comprising an insulator sequence (e.g., cHS4) and an ocular gene-encoding sequence, or (iv) a nucleic acid vector (or pharmaceutical composition thereof) comprising a promoter comprising any one of SEQ ID NOs: 3-5 and a sequence encoding MYO7A; or (v) a synthetic circular DNA vector (or pharmaceutical composition thereof) comprising a therapeutic protein-encoding sequence (e.g., an ocular gene-encoding sequence) and an S/MAR sequence; or (vi) a nucleic acid vector (or pharmaceutical composition thereof) comprising a promoter comprising the nucleotide sequence of any one of SEQ ID NOs: 6-9, or a functional variant thereof, and a sequence encoding ABCA4 (e.g., human ABCA4).

Expression of the transgene in target cells or tissues of a subject can be characterized by examining a nucleic acid sequence (e.g., an RNA sequence, e.g., an mRNA sequence) of the host cell, such as by Southern Blotting, RNA sequencing, or PCR analysis, to detect or quantify the presence (e.g., persistence) of the therapeutic sequence or transgene delivered. Alternatively, expression of the therapeutic sequence or transgene in the subject can be characterized (e.g., quantitatively or qualitatively) by monitoring the progress of a disease being treated by delivery of the therapeutic sequence (e.g., associated with a defect or mutation targeted by the therapeutic sequence). In some embodiments, transcription or expression (e.g., persistent transcription or persistent expression) of the therapeutic sequence or transgene is confirmed by observing a decline in one or more symptoms associated with the disease.

Accordingly, the invention provides methods of treating a disease in a subject by administering to the subject any of the therapeutic nucleic acid vectors of this disclosure, or pharmaceutical compositions thereof. The DNA vectors or pharmaceutical compositions thereof can be administered to a subject in a dosage from 1 μg to 10 mg of DNA (e.g., from 5 μg to 5.0 mg, from 10 μg to 2.0 mg, or from 100 μg to 1.0 mg of DNA, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of DNA, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of DNA).

In some instances, the therapeutic circular DNA vectors, and pharmaceutical compositions thereof, provided herein are amenable to repeat dosing due to their ability to transfect target cells without triggering an immune response or inducing a reduced immune response relative to a reference vector, such as a plasmid DNA vector or an AAV vector. Thus, the invention provides methods of repeatedly administering the therapeutic circular DNA vectors and pharmaceutical compositions described herein. Any of the aforementioned dosing quantities may be repeated at a suitable frequency and duration, in some embodiments, the subject receives a dose about once per month, about once every six weeks, about once every two months, about once every three months, about once every four months, twice per year, once yearly, or less frequently (e.g., once every two years or once every five years). In some embodiments, the number and frequency of doses corresponds with the rate of turnover of the target cell. It will be understood that in long-lived post-mitotic target cells transfected using the vectors described herein, a single dose of vector may be sufficient to maintain expression of the heterologous gene within the target cell for a substantial period of time. Thus, in other embodiments, a therapeutic circular DNA vector provided herein may be administered to a subject in a single dose. The number of occasions in which a therapeutic circular DNA vector is delivered to the subject can be that which is required to maintain a clinical (e.g., therapeutic) benefit.

Methods of the invention include administration of a nucleic acid vector or pharmaceutical composition thereof, through any suitable route. The nucleic acid vector, or pharmaceutical composition thereof, can be administered systemically or locally, e.g., ocularly (e.g., subretinally, intravitreally, suprachoroidally, by eye drop, intraocularly, intraorbitally), peri-ocularly (e.g., into the ciliary muscle or another peri-ocular tissue), intravenously, intramuscularly, intravitreally (e.g., by intravitreal injection), intradermally, intrahepatically, intracerebrally, intramuscularly, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, orally, topically, transdermally, by inhalation, by aerosolization, by injection (e.g., by jet injection), by electroporation, by implantation, by infusion (e.g., by continuous infusion), by localized perfusion bathing target cells directly, by catheter, by lavage, in creams, or in lipid compositions.

Any suitable means of ocular administration known in the art or described herein may be used as part of the methods provided herein. Methods of delivering a nucleic acid vector, or pharmaceutical composition thereof, to a target retinal cell include administering the nucleic acid vector, or composition thereof, to the eye by intraocular injection (e.g., injection to the posterior of the eye or the anterior of the eye by, e.g., subretinal injection, suprachoroidal injection, intravitreal injection, periocular injection, sub-tenton injection, posterior juxtascleral injection, intracameral injection, subconjunctival injection, or retrobulbar injection) or intraocular implant. In some embodiments of any of the methods described herein, the administration of the DNA vector is via an intraocular implant (e.g., a controlled release or depot implant, an intravitreal implant, a subconjunctival implant, or an episcleral implant) in other embodiments, the administration of the DNA vector is not via an intraocular implant (e.g., a controlled release or depot implant, an intravitreal implant, a subconjunctival implant, or an episcleral implant). In some embodiments of any of the methods described herein, the administration of the DNA vector is via iontophoresis (e.g., the method includes administration of the nucleic acid vector to the intraocular space by iontophoresis and subsequent delivery to the retina by transmitting a current through an electrode contacting an interior region of the eye).

In some instances, administration of the nucleic acid vector, or pharmaceutical composition thereof, is non-surgical. For example, in some embodiments, administration of the nucleic acid vector, or pharmaceutical composition thereof, does not utilize general anesthesia and/or does not involve retrobulbar anesthesia (i.e., retrobulbar block). Additionally, or alternatively, administration of the nucleic acid vector, or pharmaceutical composition thereof, does not involve injection using a needle larger than 28 gauge. Additionally, or alternatively, administration of the nucleic acid vector, or pharmaceutical composition thereof, does not involve use of a guidance mechanism that is typically required for ocular drug delivery via shunt or cannula.

In some instances, administration of the nucleic acid vector, or pharmaceutical composition thereof, is by injection (e.g., microneedle injection) into an outer tissue of the eye, e.g., the suprachoroidal space, sclera, cornea, corneal stroma, conjunctiva, subconjunctival space, or subretinal space. Alternatively, administration of the DNA vector is by injection (e.g., microneedle injection) into a site proximal to the outer tissue, such as the trabecular meshwork, ciliary body, aqueous humor or vitreous humor.

Microneedles for injecting a nucleic acid vector, or pharmaceutical composition thereof, to an eye include hollow microneedles, which may include an elongated housing for holding the proximal end of the microneedle. Microneedles may further include a means for conducting a drug formulation therethrough. For example, the means may be a flexible or rigid conduit in fluid connection with the base or proximal end of the microneedle. The means may also include a pump or other devices for creating a pressure gradient for inducing fluid flow through the device. The conduit may in operable connection with a source of the drug formulation. The source may be any suitable container. In one embodiment, the source may be in the form of a conventional syringe. The source may be a disposable unit, dose container. In one embodiment, the microneedle has an effective length of about 50 μm to about 2000 μm. In another particular embodiment, the microneedle has an effective length of from about 150 μm to about 1500 μm, from about 300 μm to about 1250 μm, from about 500 μm to about 1250 μm, from about 500 μm to about 1500 μm, from about 600 μm to about 1000 μm, or from about 700 μm to about 1000 μm. In one embodiment, the effective length of the microneedle is about 600 μm, about 700 μm, about 800 μm or about 1000 μm. In various embodiments, the proximal portion of the microneedle has a maximum width or cross-sectional dimension of from about 50 μm to 600 μm, from about 50 μm to about 400 μm, from about 50 μm to about 500 μm, from about 100 μm to about 400 μm, from about 200 μm to about 600 μm, or from about 100 μm to about 250 μm, with an aperture diameter of about 5 μm to about 400 μm. In a particular embodiment, the proximal portion of the microneedle has a maximum width or cross-sectional dimension of about 600 μm. In various embodiments, the microneedle has a bevel height from 50 μm to 500 μm, 100 μm to 500 μm, 100 μm to 400 μm, 200 μm to 400 μm, or 300 μm to 500 μm.

In particular instances, administration of the nucleic acid vector, er pharmaceutical composition thereof, is by suprachoroidal injection, which can be accomplished in a minimally invasive, non-surgical manner. For instance, suprachoroidal injection can provide nucleic acid delivery over a larger tissue area and to less accessible target tissues in a single administration as compared to other types of administration (e.g., subretinal injection). Without wishing to be bound by theory, upon entering the suprachoroidal space, a pharmaceutical composition can flow circumferentially toward the retinochoroidal tissue, macula, and optic nerve in the posterior segment of the eye. In addition, a portion of the infused pharmaceutical composition may remain in the suprachoroidal space as a depot, or remain in tissue overlying the suprachoroidal space, for example the sclera, near the microneedle insertion site, serving as additional depot of the pharmaceutical composition that can subsequently diffuse into the suprachoroidal space and into other adjacent posterior tissues.

Suprachoroidal injection can be performed using any suitable method known in the art or described herein. For example, in some instances, the nucleic acid vector is suprachoroidally administered through a microneedle (e.g., a hollow microneedle). In some instances, the nucleic acid vector is suprachoroidally administered through a microneedle array. Exemplary microneedles suitable for use in suprachoroidal administration of nucleic acid vectors described herein are described, e.g., in U.S. 2017/0273827, which is incorporated herein by reference.

Suprachoroidal injection can be performed using methods known in the art. For example, a microneedle tip can be placed into the eye so that the drug formulation flows into the suprachoroidal space and to the posterior ocular tissues surrounding the suprachoroidal space. In one embodiment, insertion of the microneedle is in the sclera of the eye. In one embodiment, drug flow into the suprachoroidal space is achieved without contacting underlying tissues with the microneedle, such as choroid and retina tissues. In some embodiments, the one or more microneedles are inserted perpendicularly, or at an angle from 80 to 100, into the eye, e.g., into the sclera, reaching the suprachoroidal space in a short penetration distance. Exemplary methods suitable for use in suprachoroidal administration of nucleic acid vectors described herein are described, e.g., in WO 2014/074823, which is incorporated herein by reference.

In some instances, the present methods of delivering a nucleic acid vector or pharmaceutical composition thereof, involve administration intravitreally. For instance, contemplated herein are intravitreal injection methods involving the InVitria Injection Assistant (FCI Ophthalmics, Pembroke, MA), Rapid Access Vitreal Injection (RAVI) Gude (Katalyst Surgical, Chesterfield, MO), Doi-Umeatsu Intravitreal Injection Guide (Duckworth & Kent Ltd., England), Malosa Intravitreal Injection Guide (Beaver-Visitec International, Waltham, MA), or automated injection guides.

Any suitable dose of nucleic acid vector, or pharmaceutical composition thereof, may be administered. For instance, in embodiments involving subretinal administration of naked nucleic acid vector, each eye may be injected with one or more blebs (e.g., two blebs per eye) each having a volume from 20-500 μL (e.g., from 50-250 μL; e.g., 50-100 μL, 100-150 μL, 150-200 μL, or 200-250 μL; e.g., about 50 μL, about 75 μL, about 100 μL, about 150 μL, or about 200 μL), e.g., one bleb, two blebs, three blebs, four blebs, or more, per eye. In embodiments involving subretinal administration of naked nucleic acid vector (e.g., a naked circular DNA vector (e.g., a synthetic and/or supercoiled circular DNA vector)), the injection volume (e.g., pharmaceutical composition) contains the nucleic acid vector at a concentration from 0.5 mg/mL to 5 mg/mL (e.g., from 1.0 mg/mL to 2.5 mg/mL; e.g., from 0.5 mg/mL to 1.0 mg/mL, from 1.0 mg/mL to 1.5 mg/mL, from 1.5 mg/mL to 2.0 mg/mL, from 2.0 mg/mL to 2.5 mg/mL, from 2.5 mg/mL to 3.0 mg/mL, from 3.0 mg/mL to 4.0 mg/mL, or from 4.0 mg/mL to 5.0 mg/mL; e.g., about 0.5 mg/mL, about 1.0 mg/mL, about 1.5 mg/mL, about 2.0 mg/mL, about 2.5 mg/mL, about 3.0 mg/mL, about 4.0 mg/mL, or about 5.0 mg/mL. In particular instances (e.g., wherein naked nucleic acid vector is administered subretinally), the injection volume (e.g., pharmaceutical composition) contains the nucleic acid vector at a concentration of 1.5 mg/mL. In some embodiments involving subretinal administration, naked nucleic acid vector is administered to each eye in an amount from 20 μg to 2.0 mg (e.g., from 100 μg to 1.0 mg, or from 200 μg to 500 μg, e.g., from 20 μg to 50 μg, from 50 μg to 100 μg, from 100 μg to 150 μg, from 150 g to 200 μg, from 200 μg to 250 μg, from 250 jig to 300 μg, from 300 μg to 350 μg, from 350 μg to 400 μg, from 400 μg to 500 jig, from 500 μg to 750 μg, from 750 μg to 1.0 mg, from 1.0 mg to 1.5 mg, or from 1.5 mg to 2.0 mg; e.g., about 20 μg, about 25 μg, about 30 μg, about 40 μg, about 50 μg, about 60 μg, about 70 μg, about 75 μg, about 80 μg, about 90 μg about 100 μg, about 125 μg, about 150 μg, about 175 μg, about 200 μg, about 225 μg, about 250 μg, about 275 μg, about 300 μg, about 350 μg, about 400 pig, about 500 μg, about 600 μg, about 700 μg, about 800 μg, about 900 μg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg). In some embodiments involving subretinal administration, naked nucleic acid vector is administered to each eye in an amount from 10⁸ to 10¹⁵ vector copies (e.g., DNA vector molecules, e.g., circular DNA vector molecules) (e.g., from 10⁸ to 10⁹, from 10⁹ to 10¹⁰, from 10¹⁰ to 10¹¹, from 10¹¹ to 10¹², from 10¹² to 10¹³, from 10¹³ to 10¹⁴, or from 10¹⁴ to 10¹⁵ vector copies; e.g., about 1×10¹¹ vector copies, about 5×10¹¹ vector copies, about 1×10¹² vector copies, about 5×10¹² vector copies, about 1×10¹³ vector copies, about 2.5×10¹³ vector copies, or about 5×10¹³ vector copies). In particular embodiments, naked nucleic acid vector is administered subretinally (e.g., in two 75 μL-blebs per eye) at a total dose per eye of about 2.5×10¹³ vector copies. In other embodiments, naked nucleic acid vector is administered subretinally (e.g., in two 75 μL-blebs per eye) at a total dose per eye of about 5×10¹² vector copies. In other embodiments, naked nucleic acid vector is administered subretinally (e.g., in two 75 μL-blebs per eye) at a total dose per eye of about 5×10¹¹ vector copies.

Nucleic acid vectors described herein can be delivered into cells via in vivo electrotransfer (e.g., in vivo electroporation), e.g., by transmitting electrical energy into the tissue in which the target ocular cell resides. Such methods involve electrotransfer of the nucleic acid vector from the extracellular space in the posterior of the eye (e.g., the suprachoroidal space, choroid, retina, or vitreous) into the target ocular cell (e.g., retinal cell). For example, in some instances in which an individual is being treated for a retinal disease or disorder, the method involves transmitting electrical energy into the retina to cause electrotransfer of the nucleic acid vector from the extracellular space of the retina into one or more retinal cell types (e.g., a photoreceptor and/or a RPE cell).

In some instances, an electrode is positioned within the interior of the individual's eye, and an electric field is transmitted through the electrode into a target ocular tissue (e.g., retina at conditions suitable for electrotransfer of the therapeutic agent (e.g., nucleic acid vector) into the target cell (e.g., target retinal cell). An electric field (e.g., a pulsed electric field (PEF)) transmitted into a target ocular tissue can promote transfer of a nucleic acid vector (e.g., a nonviral DNA vector (e.g., a naked DNA vector), a circular DNA vector into a target ocular cell. Such electrotransfer can occur through any one of several mechanisms (and combinations thereof), including electrophoresis, electrokinetically driven drug uptake, and/or electroporation. Transmission of electric fields involve conditions suitable for such mechanisms. Suitable means of generating electric fields for electrotransfer of nucleic acids in mammalian tissue are known in the art, and any suitable means known in the art or described herein can be adapted for use as part of the present invention.

Various means of generating and transmitting an electric field into a tissue are contemplated herein as part of the present methods. Devices and systems having electrodes suitable for transmitting electric fields in mammalian tissues are commercially available and can be useful in the methods disclosed herein. In some instances, the electric field is transmitted through an electrode comprising a needle (e.g., a needle positioned within the vitreous humor or in the subretinal space). Suitable needle electrodes include CLINIPORATOR® electrodes marketed by IGEA® and needle electrodes marketed by AMBU® Electrodes (e.g., needle electrodes) can be made from any suitable conductive material, such as metal or metal alloy, such as platinum, stainless steel, nickel, titanium, and combinations thereof, such as platinum/iridium alloy or nitinol.

In some embodiments, the electrode used as part of methods described herein is a substantially planar electrode, such as any of the substantially planar electrodes described in PCT/US20221021209, the disclosure of which are hereby incorporated by reference in its entirety. In some embodiments, the electrode used as part of methods described herein is a substantially planar electrode as described in PCT/US2022/021209, the disclosure of which are hereby incorporated by reference in its entirety. Such substantially planar electrodes are composed of a shape memory material (e.g., a shape memory alloy) that allows the structure of an elongate conductor (e.g., a wire electrode) to relax into a preformed, substantially planar electrode when unsheathed. The substantially planar electrode is approximately perpendicular to the longitudinal axis of the sheath and/or the proximal portion of the wire (e.g., the region that does not include the substantially planar electrode).

Electrodes (e.g., a substantially planar electrodes or a non-substantially planar electrodes (e.g., substantially axial wire electrodes)) for use in the present methods may be monopolar. In some embodiments invoking electrotransfer using a monopolar electrode, a ground electrode is attached to the individual (e.g., attached to the skin of an individual) at a point other than the eye. In some embodiments, the ground electrode is a pad contacting the skin of the buttocks, leg, torso, neck (e.g., the posterior of the neck), or head (e.g., the posterior of the head) of the individual. In some embodiments, the monopolar electrode transmits electrical energy upon becoming positively charged. In some embodiments, the monopolar electrode transmits electrical energy upon becoming negatively charged.

Alternatively, electrodes may be bipolar (e.g., a substantially planar electrodes or a non-substantially planar electrodes may be bipolar (e.g., substantially axial wire electrodes may be bipolar). In a bipolar embodiment, an auxiliary electrode may be in electrical communication with the primary electrode (e.g., substantially planar electrode or a non-substantially planar electrode (e.g., substantially axial wire electrode)). The auxiliary electrode may be proximal to the primary electrode (i.e., closer to the operator), e.g., part of, or connected to, a sheath housing a primary wire electrode. In some embodiments involving electrotransfer using a bipolar electrode, electrical energy (e.g., current) is transmitted upon application of a positive voltage to the primary electrode and a negative voltage to the auxiliary electrode. In some embodiments involving electrotransfer using a bipolar electrode, electrical energy (e.g., current) is transmitted upon application of a negative voltage to the primary electrode and a positive voltage to the auxiliary electrode.

In some instances, methods of the invention involve contacting an electrode (e.g., a substantially planar electrode or a non-substantially planar electrode (e.g., a substantially axial wire electrode)) to an interior region of the eye such that electrical energy transmitted from the electrode is sufficient to cause electrotransfer at the target tissue (e.g., the retina, e.g., the macula). Thus, methods of the invention may include positioning the electrode into electrical communication with the target tissue (e.g., retina, e.g., the macula). In particular instances, the interior region of the eye contacting the electrode includes the vitreous humor For example, the electrode may be positioned wholly or partially within the vitreous humor upon transmission of the electric field. In instances in which the electrode is positioned within the vitreous humor (e.g., wholly within the vitreous humor), the electrode may be positioned in electrical communication with the interface of the vitreous humor with the retina (e.g., an interface at the macula).

In any of the aforementioned embodiments, the proximity of the electrode (e.g., a substantially planar electrode or the tip of a needle electrode) to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy).

It will be appreciated that a variety of suitable electrical parameters and algorithms thereof may be used. The voltage source may be configured to generate an electric field strength, e.g., at a target cell, from about 10 V/cm to about 1,500 V/cm (e.g., from about 10 V/cm to about 100 V/cm, e.g., about 10 V/cm, 20 V/cm, 30 V/cm, 40 V/cm, 50 V/cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the voltage source is be configured to generate an electric field strength, e.g., at a target cell, from about 10 V/cm to about 1,000 V/cm (e.g., from about 10 V/cm to 500 V/cm or from about 500 V/cm to about 1,000 V/cm). In some embodiments, the field strength is from 50 V/cm to 300 V/cm. In some embodiments, the field strength is about 100 V/cm at the target cell.

In some embodiments, the voltage (e.g., potential) at the target cell is from 5 V to 100 V (e.g., from 10 V to 80V, from 15 V to 70 V, from 20 V to 60 V, or from 30 V to 50 V: e.g., about 10 V, about 15 V, about 20 V, about 25 V, about 30 V, about 35 V, about 40 V, about 45 V, about 50 V, about 55 V, about 60 V, about 65 V, about 70 V). In some embodiments, the voltage (e.g., potential) at the target cell is from 20 V to 60 V. In some embodiments, the voltage (e.g., potential) at the target cell is from 30 V to 50 V, e.g., about 35 V to 45 V. In any of the aforementioned embodiments, close proximity of the electrode to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy). For instance, a 40 V amplitude pulse from a monopolar intravitreal electrode positioned near the retina may result in a voltage (e.g., potential) of 35 V at a target retinal cell. It will be understood that waveform amplitudes required to achieve a given voltage at a target cell will depend on the electrode configuration (e.g., monopolar vs bipolar), electrode shape, distance between electrode and the target cell, and material properties (e.g., conductivity) of the tissue (e.g., vitreous and retina).

In some embodiments, the current resulting from the pulsed electric field is from 10 μA to 1 A (e.g., from 10 μA to 500 mA, from 10 μA to 200 mA, from 10 μA to 100 mA, from 10 A to 50 mA, or from 10 μA to 25 mA; e.g., from 50 μA to 500 mA, from 100 μA to 200 mA, or from 1 mA to 100 mA; e.g., from 10 μA to 20 μA, from 20 μA to 30 μA, from 30 A to 50 μA, from 50 μA to 100 μA, from 100 μA to 150 μA, from 150 μA to 200 μA, from 200 μA to 300 μA, from 300 μA to 400 μA, from 400 μA to 500 μA, from 500 μA to 600 μA, from 600 μA to 800 μA, from 800 μA to 1 mA, from 1 mA to 10 mA, from 10 mA to 20 mA, from 20 mA to 30 mA, from 30 mA to 40 mA, from 40 mA to 50 mA, from 50 mA to 60 mA, from 60 mA to 70 mA, from 70 mA to 80 mA, from 80 mA to 90 mA, from 90 mA to 100 mA, from 100 mA to 200 mA, from 200 mA to 300 mA, from 300 mA to 500 mA, or from 500 mA to 1 A, e.g., about 1 mA, about 5 mA, about 10 mA, about 15 mA, about 20 mA, about 25 mA, about 30 mA, about 35 mA, about 40 mA, about 45 mA, about 50 mA, about 60 mA, about 70 mA, about 80 mA, about 90 mA, or about 100 mA).

In some embodiments, the pulses of electrical energy have an amplitude of about 20 V. In some embodiments in which the pulses of electrical energy have an amplitude of about 20 V, the current is between 5 mA and 50 mA (e.g., from 10 mA to 40 mA, e.g., from 5 mA to 10 mA, from 10 mA to 15 mA, from 15 mA to 20 mA, from 20 mA to 30 mA, or from 40 mA to 50 mA). In some embodiments, the pulses of electrical energy have an amplitude of about 40 V. In some embodiments in which the pulses of electrical energy have an amplitude of about 40 V, the current is between 10 mA and 100 mA (e.g., from 20 mA to 80 mA, or from 30 mA to 70 mA, e.g., from 10 mA to 20 mA, from 20 mA to 30 mA, from 30 mA to 40 mA, from 40 mA to 50 mA, from 50 mA to 60 mA, from 60 mA to 70 mA, from 70 mA to 80 m A, from 80 mA to 90 mA, or from 90 mA to 100 mA).

In some embodiments, the electrode is positioned within about 10 mm (e.g., within 9 mm, 8 mm, 7 mm, 6 mm, 5 mm, 4.5 mm, 4 mm, 3.5 mm, 3 mm, 2.5 mm, 2 mm, 1.5 mm, 1 mm, or 0.5 mm, 0.45 mm, 0.40 mm, 0.35 mm, 0.30 mm, 0.25 mm, 0.20 mm, 0.15 mm, or 0.10 mm) of the retinal interface. The electrode may be from 0.1 to about 0.5 mm (e.g., about 0.15 mm, 0.2 mm, 0.25 mm, 0.30 mm, 0.35 mm, 0.40 mm, 0.45 mm, or 0.5 mm), or from about 0.5 mm to 5 mm (e.g., about 0.5 mm, 1 mm, 1.5 mm, 2 mm, 2.5 mm, 3 mm, 3.5 mm, 4 mm, 4.5 mm, or 5 mm) from the retinal interface upon transmission of the one or more pulses. In some embodiments, the electrode (e.g., substantially planar electrode) is within about 1 mm from the retinal interface upon transmission of the one or more pulses.

The target cell (e.g., the target retinal cell, which may be a retinal cell in the macula) may be within about 5 mm (e.g., 4.5 mm, 4 mm, 3.5 mm, 3 mm, 2.5 mm, 2 mm, 1.5 mm, 1 mm, or 0.5 mm) from the retinal interface (e.g., at the macula). For example, the target cell may be from about 0.01 mm to about 1 mm (e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 09 mm, or 1 mm) from the retinal interface.

It will be appreciated that a variety of suitable electrical parameters and algorithms thereof may be used. The voltage source may be configured to generate an electric field strength, e.g., at a target cell (e.g., a retinal cell), from about 10 V/cm to about 1,500 V/cm (e.g., from about 10 V/cm to about 100 V/cm, e.g., about 10 V/cm, 20 V/cm, 30 V/cm, 40 V/cm, 50 V % cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the voltage source is be configured to generate an electric field strength, e.g., at a target cell (e.g., a retinal cell), from about 10 V/cm to about 1,000 V/cm (e.g., from about 10 V/cm to 500 V/cm or from about 500 V/cm to about 1,000 V/cm). In some embodiments, the field strength is from 50 V/cm to 300 V/cm. In some embodiments, the field strength is about 100 V/cm at the target cell (e.g., the target retinal cell).

In some embodiments, the total number of pulses of electrical energy are delivered within 1-60 seconds (e.g., within 1-5 seconds, 5-10 seconds, 10-15 seconds, 15-20 seconds, 20-30 seconds, 30-40 seconds, 40-50 seconds, or 50-60 seconds). In some embodiments, the total number of pulses of electrical energy are delivered within 1-20 seconds. For example, the total number of pulses of electrical energy may be delivered within 1-5 seconds, 5-10 seconds, 10-15 seconds, or 15-20 seconds, e.g., 1, 2, 3, 4, 5, 6, 7, 8, 9, 10, 11, 12, 13, 14, 15, 16, 17, 18, 19, or 20 seconds. The pulses of electrical energy may be, e.g., square waveforms. The pulses of electrical energy may have an amplitude from 5 V to 500 V. For example, the pulses of electrical energy may have an amplitude of about 5 V, 10 V, 15 V, 20 V, 25 V, 30 V, 35V, 40 V, 45 V, 50 V, 60 V, 70 V, 80 V, 90 V, 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of about 5-250 V (e.g., about 20 V). Any of the aforementioned voltages can be the tops of square-waveforms, peaks in sinusoidal waveforms, peaks in sawtooth waveforms, root mean square (RMS) voltages of sinusoidal waveforms, or RMS voltages of sawtooth waveforms.

In some embodiments, about 1-12 pulses (e.g., 1, 2, 3, 4, 5, 6, 7, 8, 9, 10, 11, or 12 pulses) of electrical energy are transmitted during use in some embodiments, about 4-12 pulses of electrical energy are transmitted during use.

In some embodiments, each of the pulses is from about 0.01 ms to about 200 ms in duration, from about 0.1 ms to about 200 ms in duration, or from about 1 ms to about 200 ms in duration (e.g., 0.10 ms to about 200 ms in duration. For example, each of the pulses may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms). In some embodiments, each of the pulses is about 20 ms in duration. In some embodiments, each of the pulses is about 50 ms in duration. In some embodiments, each of the pulses is from about 0.01 ms to about 1 ms (e.g., from 0.01 ms to 0.05 ms, from 0.05 ma to 0.1 ms, from 0.1 ms to 0.25 ms, from 0.25 ms to 0.5 ms, from 0.5 ms to 0.75 ms, or from 0.75 ms to 1.0 ms; e.g., about 0.01 ms, about 0.05 ms, about 0.1 ms, about 0.2 ms, about 0.3 ms, about 0.4 ms, about 0.5 ms, about 0.6 ms, about 0.7 ms, about 0.8 ms, about 0.9 ms, or about 1.0 ms) in duration.

In some embodiments, each of the pulses of electrical energy is from about 10 ms to about 200 ms. For example, each of the pulses of electrical energy may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms. In some embodiments, each of the pulses of electrical energy is from about 50 ms. In some embodiments, each of the pulses of electrical energy is less than 10 ms. For example, each of the pulses of electrical energy may be from about 10 μs to about 10 ms, e.g., from about 10 μs to about 100 μs, e.g., about 20 μs, 30 μs, 40 μs, 50 μs, 60 μs, 70 μs, 80 μs, 90 μs, or 100 μs, e.g., from about 100 μs to about 1 ms, e.g., about 200 μs, 300 μs, 400 μs, 500 μs, 600 μs, 700 μs, 800 μs, 900 μs, or 1 ms, e.g., from about 1 ms to about 10 ms, e.g., about 2 ms, 3 ms, 4 ms, 5 ms, 6 ms, 7 ms, 8 ms, 9 ms, or 10 ms.

In some embodiments, the total number of pulses of electrical energy are transmitted within 1-20 seconds (e.g., within 6-12 seconds, e.g., within 1-3 seconds, within 3-6 seconds, within 6-10 seconds, within 10-15 seconds, or within 15-20 seconds, e.g., within one second, within two seconds, within three seconds, within four seconds, within five seconds, within six seconds, within seven seconds, within eight seconds, within nine seconds, within ten seconds, within 11 seconds, within 12 seconds, within 13 seconds, within 14 seconds, within 15 seconds, within 16 seconds, within 17 seconds, within 18 seconds, within 19 seconds, within 20 seconds). The pulses of electrical energy may have an amplitude from 5 V to 1,500 V. For example, the pulses of electrical energy may have an amplitude from about 5 V to 500 V, from about 500 V to about 1,000 V, or from about 1,000 V to about 1,500 V. In some embodiments, the pulses of electrical energy have an amplitude from 5 V to 500 V. For example, the pulses of electrical energy may have an amplitude of about 5 V, 10 V, 15 V, 20 V, 25 V, 30 V, 40 V, 50 V, 100 V, 125 V, 150 V, 175 V, 200 V, 225 V, 250 V, 275 V, 300 V, 325 V, 350 V, 375 V, 400 V, 425 V, 450 V, 475 V, or 500 V. In some embodiments, the pulses of electrical energy have an amplitude of from about 5 V to about 250 V.

An electric field suitable for electrotransfer can be transmitted to a target ocular cell at or near the time of administration of the nucleic acid vector or pharmaceutical composition thereof (e.g., as part of the same procedure). For example, the present invention includes methods in which an electric field is transmitted within one hour of administration of the nucleic acid vector or pharmaceutical composition thereof (e.g., within 55 minutes, within 50 minutes, within 45 minutes, within 40 minutes, within 35 minutes, within 30 minutes, within 25 minutes, within 20 minutes, within 15 minutes, within 10 minutes, within 5 minutes, within 4 minutes, within 3 minutes, within 2 minutes, within 90 seconds, within 60 seconds, within 45 seconds, with 30 seconds, within 20 seconds, within 15 seconds, within 10 seconds, within 9 seconds, within 8 seconds, within 7 seconds, within 6 seconds, within 5 seconds, within 4 seconds, within 3 seconds, within 2 seconds, or within 1 second) of administration of the nucleic acid vector or pharmaceutical composition thereof (e.g., simultaneously with administration of the nucleic acid vector or pharmaceutical composition thereof or after administration but within any of the aforementioned durations). In some embodiments, an electric field is transmitted within 24 hours of administration of the nucleic acid vector or pharmaceutical composition thereof (e.g., within 22 hours, within 20 hours, within 18 hours, within 16 hours, within 14 hours, within 12 hours, within 10 hours, within 8 hours, within 6 hours, within 5 hours, within 4 hours, within 3 hours, within 2 hours, within 90 minutes, within 60 minutes, within 45 minutes, within 30 minutes, within 20 minutes, within 15 minutes, within 10 minutes, within 8 minutes, within 6 minutes, within 5 minutes, within 4 minutes, within 3 minutes, or within 2 minutes) of administration of the nucleic acid vector or pharmaceutical composition thereof. In some embodiments, an electric field is transmitted within 7 days of administration of the nucleic acid vector or pharmaceutical composition thereof (e.g., within 6 days, within 5 days, within 4 days, within 3 days, or within 2 days) of administration of the nucleic acid vector or pharmaceutical composition thereof.

An electric field suitable for electrotransfer can be transmitted at or near the site of administration (e.g., injection) of the nucleic acid vector or pharmaceutical composition thereof. For instance, in some embodiments, the nucleic acid vector or pharmaceutical composition thereof is administered intravitreally, and the electrode is positioned at or near the site of intravitreal administration for transmission of the electric field (e.g., within 10 mm, within 8 mm, within 6 mm, within 5 mm, within 4 mm, within 3 mm, within 2 mm, or within 1 mm of the site of intravitreal administration). In other embodiments, the nucleic acid vector or pharmaceutical composition thereof is administered (e.g., injected) subretinally, and the electrode is positioned at or near the site of subretinal administration for transmission of the electric field (e.g., within 10 mm, within 8 mm, within 6 mm, within 5 mm, within 4 mm, within 3 mm, within 2 mm, or within 1 mm of the site of subretinal administration). In other embodiments, the nucleic acid vector or pharmaceutical composition thereof is administered suprachoroidally, and the electrode is positioned at or near the site of suprachoroidal administration for transmission of the electric field (e.g., within 10 mm, within 8 mm, within 6 mm, within 5 mm, within 4 mm, within 3 mm, within 2 mm, or within 1 mm of the site of suprachoroidal administration).

In some instances, the nucleic acid vector or pharmaceutical composition thereof is administered at a location that is exposed to the electric field (or will be exposed to the electric field, in the event of subsequent electric field transmission). In some embodiments, the nucleic acid vector or pharmaceutical composition thereof is delivered at a location that is exposed to (or will be exposed to) a voltage that is 1% or more of the maximum tissue voltage (e.g., at least 5% of the maximum tissue voltage, at least 10% of the maximum tissue voltage, at least 20% of the maximum tissue voltage, at least 30% of the maximum tissue voltage, at least 40% of the maximum tissue voltage, at least 50% of the maximum tissue voltage, at least 60% of the maximum tissue voltage, at least 70% of the maximum tissue voltage, at least 80% of the maximum tissue voltage, or at least 90% of the maximum tissue voltage, e.g., from 1% to 10% of the maximum tissue voltage, from 10% to 20% of the maximum tissue voltage, from 20% to 30% of the maximum tissue voltage, from 30% to 40% of the maximum tissue voltage, from 40% to 50% of the maximum tissue voltage, from 50% to 60% of the maximum tissue voltage, from 60% to 70% of the maximum tissue voltage, from 70% to 80% of the maximum tissue voltage, from 80% to 90% of the maximum tissue voltage, from 90% to 95% of the maximum tissue voltage, or from 95% to 100% of the maximum tissue voltage).

Alternatively, the site of administration can be in a region of tissue away from the electric field. For example, administration of the nucleic acid vector or pharmaceutical composition thereof may be systemic (e.g., intravenous), while the electric field is transmitted in the eye (e.g., in the vitreous humor or in the subretinal space).

In any of the methods described herein involving electrotransfer (e.g., by PEF), a paralytic may be administered according to standard procedures, which can help reduce the risk and/or severity of muscle contractions upon transmission of electrical energy.

Additionally, or alternatively, nucleic acid vectors or pharmaceutical compositions thereof can be administered to host cells ex vivo, such as by cells explanted from an individual patient, followed by reimplantation of the host cells into a patient, e.g., after selection for cells which have incorporated the vector. Thus, in some aspects, the disclosure provides transfected host cells and methods of administration thereof for treating a disease.

The level or concentration of a protein (e.g., an ocular protein (e.g., retinal protein)) expressed from a DNA vector described herein may be an expression level, presence, absence, truncation, or alteration of the administered vector. It can be advantageous to correlate the level with one or more clinical phenotypes or with an assay for a human disease biomarker. The assay may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Therapeutic genes delivered by the nucleic acid vectors described herein may be quantified using methods such as, but not limited to, ultraviolet visible spectroscopy (UV/Vis). The quantified polynucleotide may be analyzed in order to determine if the polynucleotide may be of proper size, check that no degradation of the polynucleotide has occurred. Degradation of the polynucleotide may be checked by methods such as, but not limited to, agarose gel electrophoresis, HPLC based purification methods such as, but not limited to, strong anion exchange HPLC, weak anion exchange HPLC, reverse phase HPLC (RP-HPLC), hydrophobic interaction HPLC (HIC-HPLC), liquid chromatography-mass spectrometry (LCMS), capillary electrophoresis (CE), and capillary gel electrophoresis (COE).

Efficacy of treatment can be monitored, assessed, and/or quantified using any suitable methods known in the art or provided herein. For example, an individual treated for a retinal disease or disorder may be monitored periodically to assess progression of retinal degeneration, e.g., by testing visual acuity and visual field using standard tests. Additionally, or alternatively, optical coherence tomography (OCT)(e.g., spectral domain OCT (SD-OCT)) can be conducted to assess changes in retinal structure. In some instances, an individual treated by the methods described herein exhibits improvement or no further degradation in retinal structure assessed by imaging endpoints, such as fundus autofluorescence (FAF) and/or SD-OCT.

Safety and tolerability of treatment can be monitored, assessed, and/or quantified using any suitable methods known in the art or provided herein. For instance, an individual treated for a retinal disease or disorder may be monitored periodically to assess cataract formation, intra-ocular inflammation, or retina damage such as RPE hypopigmentation. In some embodiments, an individual treated according to the methods described herein exhibits no cataract formation, no intraocular inflammation up to two months post-treatment (or less than grade 2 intraocular inflammation up to two months post-treatment), and/or minimal retina/RPE damage (e.g., RPE hypopigmentation).

Accordingly, methods of the present invention include, after administering any of the nucleic acid vectors, or pharmaceutical compositions thereof, described herein to a subject, subsequently detecting the expression of the transgene in the subject. Expression can be detected one week to four weeks after administration, one month to four months after administration, four months to one year after administration, one year to five yeas after administration, or five years to twenty years after administration (e.g., at least one week, at least two weeks, at least one month, at least four months, at least one year, at least two years, at least five years, at least ten years after administration). At any of these detection timepoints, persistence (e.g., episomal persistence) of the nucleic acid vector may be observed. In some embodiments, the persistence of a circular DNA vector is from 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector (e.g., a circular DNA vector produced in bacteria or having one or more bacterial signatures not present in the vector of the invention).

Expression constructs and nucleic acid vectors of the invention can be expressed in a particular target cell. Methods of the invention include expressing a transgene in a target cell that would normally express the endogenous gene being augmented or replaced by the transgene. In certain instances, the transgene is expressed preferentially in the target cell (e.g., as compared to a non-target cell). For instance, a target cell may be a photoreceptor, and the methods provided herein include expressing the transgene in a photoreceptor. In some instances, the transgene is ABCA4 and the target cell is a photoreceptor In some instances, the transgene (e.g., ABCA4) is preferentially expressed in photoreceptors as compared to retinal pigment epithelial (RPE) cells (e.g., the number of ABCA4 protein molecules expressed by the ABCA4 transgene in photoreceptors exceeds the number of ABCA4 protein molecules expressed by the ABCA4 transgene in RPE cells, e.g., the number of ABCA4 protein molecules expressed by the ABCA4 transgene in photoreceptors exceeds the number of ABCA4 protein molecules expressed by the ABCA4 transgene in RPE cells by at least 20%, by at least 50%, by at least two-fold, by at least five-fold, by at least ten-fold, by at least 50-fold, by at least 100-fold, or more).

In some embodiments, a target cell may be an RPE cell (e.g., in embodiments involving expression constructs containing a MYO7A transgene), and the methods provided herein include expressing the transgene in RPE cells. In some instances, the transgene is MYO7A and the target cell is an RPE cell. In some instances, the transgene (e.g., MYO7A) is preferentially expressed in RPE cells as compared to photoreceptors or other non-target cells, such as choroid cells (e.g., the number of MYO7A protein molecules expressed by the MYO7A transgene in RPE cells exceeds the number of MYO7A protein molecules expressed by the MYO7A transgene in photoreceptor cells, e.g., the number of MYO7A protein molecules expressed by the MYO7A transgene in RPE cells exceeds the number of MYO7A protein molecules expressed by the MYO7A transgene in photoreceptors by at least 20%, by at least 50%, by at least two-fold, by at least five-fold, by at least ten-fold, by at least 50-fold, by at least 100-fold, or more).

In some embodiments of methods involving preferential expression in a target cell by administration of a nucleic acid vector, in vivo electrotransfer is performed as part of the method, or before or after the administration of the nucleic acid vector or pharmaceutical composition thereof. For example, methods involving preferential expression of ABCA4 in photoreceptor cells (compared to RPE cells) include methods of administering any of the ABCA4-encoding nucleic acid vectors described herein, or pharmaceutical composition thereof, in combination with any of the ocular electrotransfer methods described herein (e.g., using an intra-ocular electrode). In methods involving preferential expression of MYO7A in RPE cells (compared to photoreceptor cells) include methods of administering any of the MYO7A-encoding nucleic acid vectors described herein, or pharmaceutical composition thereof, in combination with any of the ocular electrotransfer methods described herein (e.g., using an intra-ocular electrode).

In some instances, an individual is treated with nucleic acid vector, or pharmaceutical composition thereof, according to any of the embodiments described herein only once in their lifetime (e.g., treatment of the disease or disorder is sustained for several years (e.g., three to five years, five to ten years, ten to fifteen years, or at least 15 years)). Alternatively, an individual may be treated exactly twice in their lifetime. Additionally, or alternatively, an individual may be treated once every 2-3 years, every 3-5 years, or every 5-10 years.

Nucleic acid vectors and pharmaceutical compositions described herein can be used for treatment of various ocular diseases or disorders. In some instances, the ocular disease or disorder is a retinal disease or disorder, such as a retinal dystrophy (e.g., a retinal dystrophy characterized by reduced level of functional expression (e.g., a lack of functional expression) of a retinal protein in the individual relative to a reference (e.g., a healthy level of functional expression)). In some embodiments, the ocular disease or disorder (e.g., retinal disease or disorder) is a monogenic disorder. In some embodiments, the ocular disease or disorder (e.g., retinal disease or disorder) is a recessively inherited disorder. In some embodiments, the individual has, or is expected to develop, an ocular disease or disorder (e.g., retinal disease or disorder) caused by a heterozygous mutation. In other embodiments, the individual has, or is expected to develop, an ocular disease or disorder (e.g., retinal disease or disorder) caused by a homozygous mutation.

In some embodiments (e.g., in embodiments in which the individual is being treated for an ABCA4-associated retinal dystrophy, e.g., Stargardt disease, cone-rod dystrophy, retinitis pigmentosa, or age-related macular degeneration), the retinal protein is ABCA4. In such embodiments, the individual may bean adult, a teenager, or a child with retinal degeneration due to ABCA4 mutation (e.g., a biallelic ABCA4 mutation). In some instances, the individual has macular degeneration due to recessive biallelic ABCA4 mutations. The individual may have retinal degeneration of any severity due to biallelic ABCA4 mutations.

In some embodiments (e.g., in embodiments in which the individual is being treated for Usher syndrome 1B), the retinal protein is MYO7A.

In some embodiments, the ocular disease or disorder is selected from the group consisting of Usher syndrome (e.g., Usher syndrome type 1B), macular degeneration (e.g., age related macular degeneration (AMD), wet macular degeneration (e.g., wet AMD), dry macular degeneration (e.g., dry AMD), or neovascular AMD), geographic atrophy, retinitis pigmentosa (RP), diabetic ocular disorders (e.g., diabetic retinopathy or diabetic macular edema), dry eye, cataracts, retinal vein occlusion (e.g., central retinal vein occlusion or branched retinal vein occlusion), retinal artery occlusion, macular edema (e.g., macular edema occurring after retinal vein occlusion), refraction and accommodation disorders, keratoconus, amblyopia, glaucoma, Stargardt disease, endophthalmitis, conjunctivitis, uveitis (e.g., posterior uveitis), retinal detachment, corneal ulcers, dacryocystitis, Duane retraction syndrome, optic neuritis, choroidal neovascularization, choroidal ischemia, or hypertensive retinopathy.

In some embodiments, the ocular disease or disorder is a retinal dystrophy (e.g., a Mendelian-heritable retinal dystrophy). In some embodiments, the retinal dystrophy is selected from the group consisting of Leber's congenital amaurosis (LCA), Stargardt Disease, pseudoxanthoma elasticum, rod cone dystrophy, exudative vitreoretinopathy, Joubert Syndrome, congenital stationary night blindness, type 1C (CSNB-1C), age-related macular degeneration, retinitis pigmentosa, stickler syndrome, microcephaly and chorioretinopathy, retinitis pigmentosa, CSNB 2, Usher syndrome, and Wagner syndrome.

In some instances, the methods provided herein are useful for treatment of symptoms of such ocular diseases or disorders, such as any of the above diseases or disorders, or ocular symptoms of broader disorders, such as hypotension, hypertension, infection, sarcoid, or sickle cell disease. In some embodiments, the disease is an acute ocular disease. In other embodiments, the disease is a chronic ocular disease.

In some embodiments, the individual to be treated is a human patient. In some embodiments, the individual is a pediatric human patient, e.g., a person aged 21 years or younger at the time of their diagnosis or treatment. In some embodiments, the pediatric human patient is aged 16 years or younger at the time of treatment. In other embodiments, the individual is aged 22 to 40 years at the time of treatment. In other embodiments, the individual is aged 41 to 60 years at the time of treatment. In other embodiments, the individual is aged 61 years or older at the time of treatment. In some instances, the individual is male. In other instances, the individual is female.

### VI. Kits and Articles or Manufacture

In another aspect of the invention, provided herein is an article of manufacture or a kit containing any of the nucleic acid vectors, or pharmaceutical compositions thereof, described herein. Thus, nucleic acid vectors contemplated herein as part of a kit or article of manufacture include (i) a nucleic acid vector including a cDNA sequence encoding ABCA4 and a regulatory element comprising a sequence derived from ABCA4 intron 6; (ii) a nucleic acid vector comprising a native ABCA4 promoter or functional variant thereof, a sequence encoding ABCA4, and a regulatory element (e.g., a regulatory element comprising a scaffold/matrix attachment region (S/MAR) sequence, or functional variant thereof, or a sequence derived from ABCA4 intron 6), (iii) a nucleic acid vector comprising a first regulatory element comprising an insulator sequence (e.g., cHS4) and an ocular gene-encoding sequence; (iv) a nucleic acid vector comprising a promoter comprising any one of SEQ ID NOs: 3-5 and a sequence encoding MYO7A; (v) a synthetic circular DNA vector comprising a therapeutic protein-encoding sequence (e.g., an ocular gene-encoding sequence) and a scaffold/matrix attachment region (S/MAR) sequence; or (vi) a nucleic acid vector comprising a promoter comprising the nucleotide sequence of any one of SEQ ID NOs 6-9, or a functional variant thereof, and a sequence encoding ABCA4 (e.g., human ABCA4).

The article of manufacture or kit can include a container and a label or package insert on, or associated with, the container. Suitable containers include, for example, bottles, vials, syringes, IV solution bags, etc. The containers may be formed from a variety of materials, such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). At least one active agent in the composition is a nucleic acid vector of the invention or a pharmaceutical composition comprising the nucleic acid vector.

The label or package insert indicates that the composition is used for treating the condition treatable by its contents (e.g., an ocular disease or disorder, e.g., an ABCA4-associated retinal dystrophy or a MYO7A-associated retinal dystrophy).

Moreover, the article of manufacture may comprise (a) a first container with a composition contained therein, wherein the composition comprises any of the nucleic acid vectors described herein; and (b) a second container with a composition contained therein, wherein the composition comprises an additional. Therapeutic agent. The article of manufacture may further comprise a package insert indicating that the compositions can be used to treat a particular condition (e.g., an ocular disease or disorder, e.g., an ABCA4-associated retinal dystrophy or a MYO7A-associated retinal dystrophy).

Alternatively, or additionally, the article of manufacture may further comprise a second (or third) container comprising a pharmaceutically acceptable carrier, such as bacteriostatic water for injection (BWFI), phosphate-buffered saline, Ringer's solution, dextrose solution, or any of the pharmaceutically acceptable carriers disclosed above. It may further include other materials desirable from a commercial and user standpoint, including other buffers, diluents, filters, needles, syringes, or other delivery devices.

### EXAMPLES

These examples are provided for illustrative purposes only and not to limit the scope of the claims provided herein.

### Example 1. ABCA4 and MYO7A Expression Construct Selection, Design, and Vector Assembly

### Step 1—Selection of Potential Expression Construct Elements

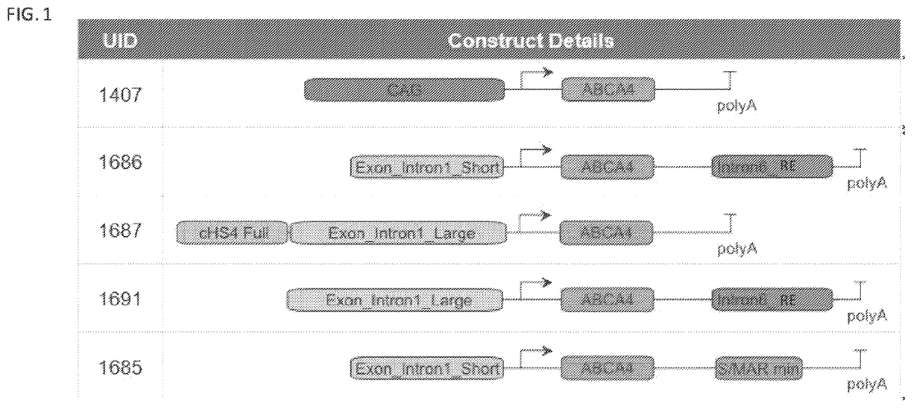

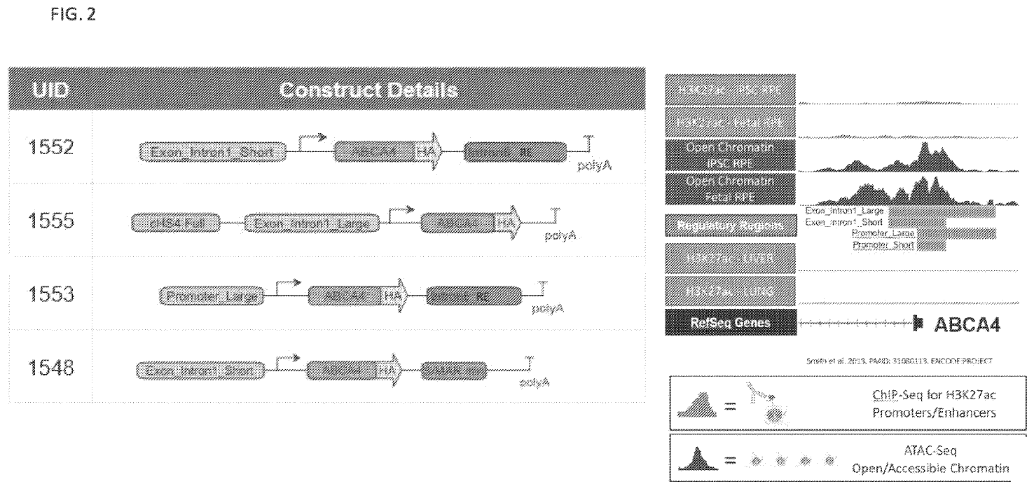

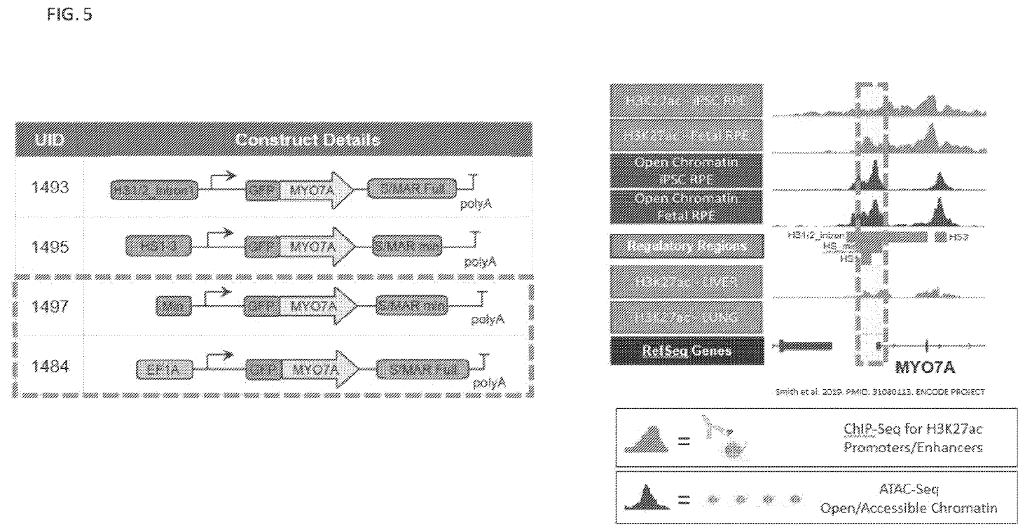

Potential native ARCA4 and MYO7A promoter constructs were selected by analyzing genomic datasets with chromatin structure data deposited in publicly available repositories. These datasets were searched for cell types of interest, e.g., ocular cells. Relevant datasets were chosen based on chromatin accessibility data, including histone post-translational modifications. Areas of enrichment for chromatin modification and/or open chromatin in the region of ABCA4 and MYO7A were identified based on these data and potential regulatory sequences were selected in sizes ranging from ˜2 kb down to a few hundred bases. The following candidate native or constitutive promoter sequences were chosen for MYO7A: MYO7A Promoter HS1/2_Intron1 (SEQ ID NO: 3). MYO7A Promoter HS1-3 (SEQ ID NO: 4), and MYO7A Promoter Min (SEQ ID NO: 5). The following candidate promoter sequences were chosen for ABCA4 ABCA4 Promoter Exon_Intron1_Short (SEQ ID NO: 6), ABCA4 Promoter Exon_Intron1_large (SEQ ID NO: 7), ABCA4 Promoter_Large (SEQ ID NO: 8), and ABCA4 Promoter_Short (SEQ ID NO: 9). Examples of ChIP-Seq for 13K27ac and ATAC-Seq (the reference for those experiments are below the images) for open/accessible chromatin are shown to illustrate how candidate regulatory elements were mapped for ABC A4 and MYO7A expression construct generation as shown in FIG. 2 and FIG. 5, respectively. Various expression constructs were constructed including various promoters and regulatory elements, as shown in FIGS. 1-7. In addition to cDNA coding sequences for ABCA4 (SEQ ID NO: 1) or MYO7A (SEQ ID NO: 2), the following elements were included in constructs as indicated in the figures: S/MAR_Full, which is the full-length human interferon-β S/MAR (SEQ ID NO: 10), S/MAR min, which includes three repeats of a portion of the human interferon-β S/MAR (SEQ ID NO: 11); MYO7A Promoter HS1/2_Intron1 (SEQ ID NO: 3); Promoter HS1-3 (SEQ ID NO: 4); MYO7A Promoter Min (SEQ ID NO: 5)); ABCA4 Intron 6 RE (regulatory element derived from a nuclease-sensitive region of ABCA4 intron 6—see Example 7 below) (SEQ ID NO: 13), ABCA4 Promoter Exon_intron1_Short (SEQ ID NO: 6), ABCA4 Promoter Exon_intron1_large (SEQ ID NO: 7), ABCA4 Promoter_Large (SEQ ID NO: 8), ABCA4 Promoter_Short (SEQ ID NO: 9), chicken β-globin insulator (cHS4: SEQ ID NO: 12); a CAG promoter (SEQ ID NO: 14), or an EF1A promoter (SEQ ID NO: 15).

### Step 2—Synthesis of Synthetic DNA Vectors Encoding ABCA4 or MYO7A

The sequences of expression construct elements described above were synthesized into plasmids using standard manufacturing processes to produce plasmids each containing a single element. These single-element plasmids were then ligated together to produce template plasmids containing all elements of an expression construct using a BsaI cloning process. Briefly, each plasmid having an individual expression element contained BsaI restriction sites flanking the element and overhangs required to ligate the sequences in the right order and orientation. The BsaI restriction reaction cut each required element from its respective plasmid and ligated the fragments into a new plasmid at the same time. For example, to make expression construct 1686 shown in FIG. 1, three plasmids having the ABCA4 Exon_Intron1_Short sequence (SEQ ID NO: 6), the ABCA4 coding sequence (SEQ ID NO: 1), and the Intron 6 RE (SEQ ID NO: 13) sequences were mixed with a plasmid with BsaI restriction sites having appropriate overhangs and subjected to BsaI restriction and ligation. The BsaI restriction overhangs were designed such that the ligation of the expression elements together would generate a plasmid having an expression construct with the elements in the order shown in FIG. 1. The BsaI restriction and ligation reaction was prepared as follows: 2 μl of BSA buffer, 2 μl of T4 ligase buffer, 1.5 μl BsaI, 0.5 μl of T4 ligase, and equimolar concentrations of each plasmid preparation and water to reach 20 μl total volume. This master mix was then briefly vortexed/mixed and briefly centrifuged. The master mix was then placed in a thermocycler with the following steps (1) 37° C. for 15 minutes; (2) 37° C. for two minutes, (3) 16° C. for five minutes; and (4) repeat steps (2) and (3) 50 times.

One μl of the resulting product was then used to transform E. coli using protocols well known in the art. Resulting plasmids were then purified and the DNA digested with the relevant restriction enzyme to verify the accuracy of the final plasmid. Positive DNA sequence clones of interest were then verified using DNA sequencing and subsequently amplified following the verification. These sequences were then cloned into a type IIs restriction site-containing backbone to form template plasmids for generation of synthetic circular DNA vectors.

Synthetic circular DNA vectors were then produced from the template plasmids using methods generally taught in International Patent Publication Number WO 2019/178500 to remove plasmid backbone components, such as bacterial origins of replication and resistance genes. Briefly, template plasmids were amplified by rolling circle amplification using Phi29 polymerase, restriction enzymes were added to cut the amplified product at sites flanking the therapeutic sequence, and the therapeutic sequence was recircularized by ligation using a ligase. In the present examples, the process was carried out using a single restriction enzyme, BsaI, which cut recognition sites flanking the therapeutic sequence and within the plasmid backbone. Upon ligation, the linear therapeutic fragment circularizes into a therapeutic circular DNA vector, and the linear backbone fragment circularizes. Without being bound by theory, the circularized backbone fragment contains a BsaI cut site and ligation occurs in the presence of the BsaI enzyme, so BsaI can cut the backbone and does not cut the therapeutic circular DNA vector, thereby driving the reaction forward toward a purer therapeutic circular DNA product. Exonuclease was added to digest the remaining linear backbone, and gyrase was added to supercoil the therapeutic circular DNA vector.

Alternatively, circular DNA vectors can be produced in bacteria, as described herein and in WO 2023/122625, which is hereby incorporated by reference in its entirety.

### Example 2. Functional Testing of ABCA4 Expression Constructs

The expression constructs of interest identified in FIGS. 1-7, were selected and the expression and persistence of these expression constructs were then screened by transfecting relevant cell types, e.g., iRPE cells, using standard techniques, and testing the copy number of the vectors and expression of the target gene, e.g., ABCA4 or MYO7A, in vitro.

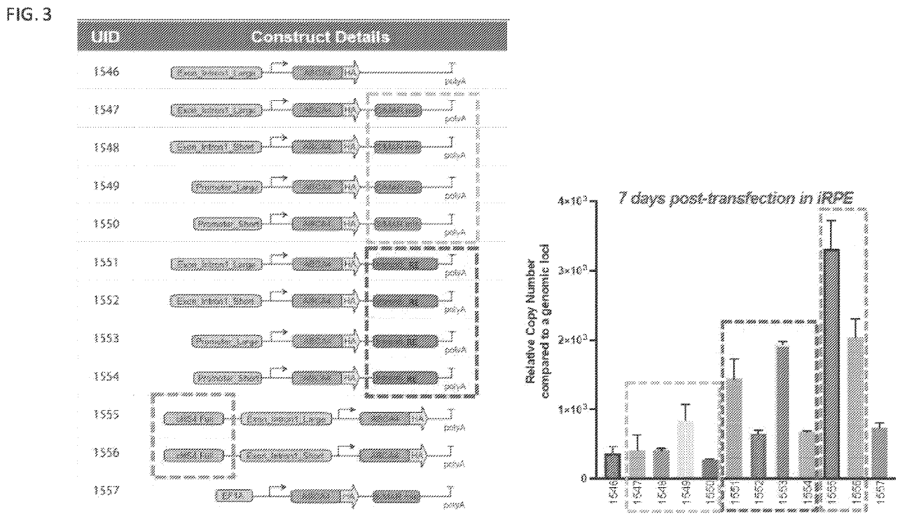

Transfection was performed with Lipofectamine 3000 following a standard protocol 300K iRPE cells were seeded in Laminin coated 6-well plates in 800 μl of media. All DNA vectors were normalized to 0.2 pmol of DNA ranging from 4-9 μg. After 24 hours, cells were washed with PBS and fresh media was added. Transfected cells were grown for seven days post-transfection. DNA and RNA were extracted following standard protocols (MONARCH® Genomic DNA Purification Kit from NEB and RNeasy Mini Kit from Qiagen). Detection by qPCR was performed with LUNA® Universal One-Step RT-qPCR Kit in a Q7 thermocycler. Transfection efficiency was assessed using DNA copy number detected by qPCR for DNA, using primers for a genomic control region and specific ABCA4 or MYO7A primers. The DNA copy number was reported relative to the genomic control region loci (FIG. 3). Primers for the genomic control amplified a region in exon 27 of the RB1 human gene were (Fwd:CCTAGCCITFAAGGGCTCTCTA (SEQ ID NO: 16); Rev:TCrACCTCTGTGAAAAGATCAGGG (SEQ ID NO: 17), and primers specific for the codon optimized ABCA4 sequence were (Fwd: TGGGAGTTAGACCCGGCGAGTG (SEQ ID NO: 18), Rev: TGTAGCATCTCCGCTTGTCACT (SEQ ID NO: 19)). Expression constructs containing a cHS4 Full insulator sequence (UID 1555 and 1556) resulted in the highest observed DNA copy number (see FIG. 3).

Gene expression was assessed by harvesting transfected cells and performing assays to detect protein and/or RNA relative expression. Protein detection was performed by standard western blot or immunofluorescence.

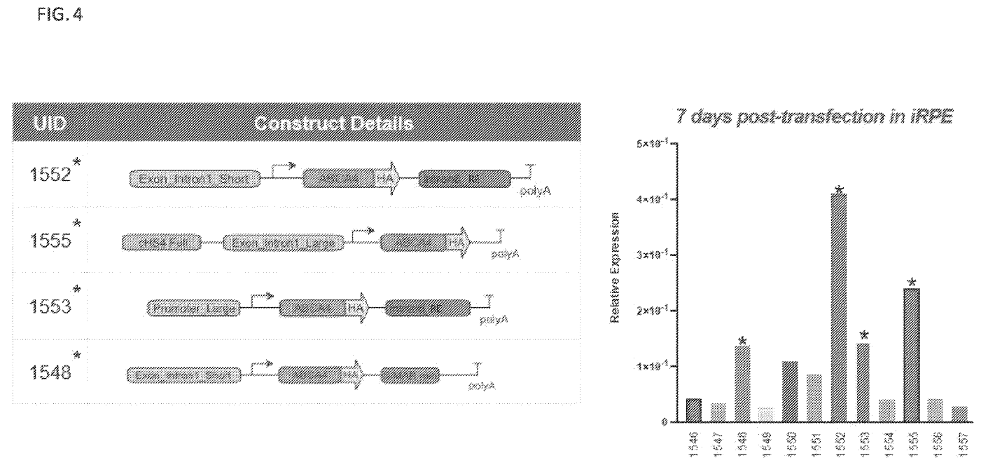

RNA expression of transfected cells was assessed by a relative quantification by RT-qPCR against a housekeeping gene, GAPDH, for selected constructs RNA expression was then normalized to copy number by DNA content qPCR using a genomic control region and plasmid specific primers. For RT-qPCR: for the codon optimized ABCA4 (same primers as above); for Human GAPDH (Fwd: CAGTCTTCTGGCTGGCAGTG (SEQ ID NO: 20); Rev: AACCATGAGAAGTATGACAACAGC (SEQ ID NO: 21)). Constructs of interest were selected for further analysis based upon the efficacy of the constructs for increasing DNA copy number and RNA expression. The ABCA4 expression constructs UID 1552, 1555, 1553, and 1548, denoted by * in FIG. 4, generated the highest relative expression of mRNA and episomal persistence seven days following transfection into iRPE cells. Results for copy number and expression for ABCA4 expression constructs are shown in FIG. 3 and FIG. 4.

### Example 3. Functional Testing of MYO7A Expression Constructs

Natural endogenous promoters for MYO7A were screened and were selected based on transcription factor binding and localization using ChIP-SEQ for H3K27ac, and ATAC-Seq for open/accessible chromatin (FIG. 5). EF1 A was also selected as a strong constitutive promoter. Representative MYO7A expression constructs are shown in FIG. 5.

Localization of the expressed proteins was assessed using neon transfection in iRPE cells iRPE cells were seeded at 1:3 to Laminin coated 6-well plate and cultured for 48 hours to 100% confluency. Cells were lifted with TrypLE and counted for cell numbers. Greater than 2 5×10³ cells were selected for one 24-well plate and resuspended in Buffer R (Thermo Scientific). Plasmid DNA was diluted to 1 μg/well in Buffer R and mixed with iRPE cells. Cells were electroporated (Neon Transfection System; 1100 V, 20 ms, 2 pulses) and seeded to a 24-well plate containing 0.5 mL conditional media and grown for at least 48 hours.

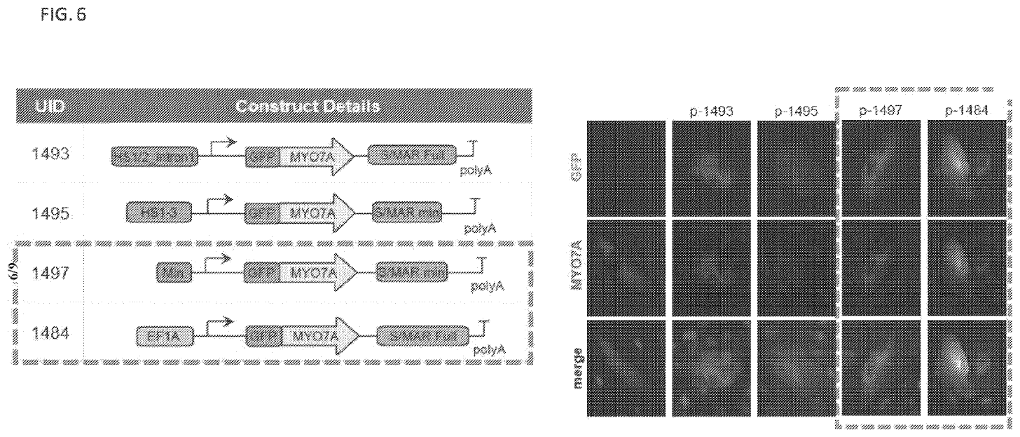

Immunocytochemistry was then performed on the transfected cells using the following protocol: Cells were fixed with 4% PFA at room temperature for 15 minutes. Cells were washed three times with PBS for five minutes and subsequently blocked with 5% BSA in 0.3 Triton-X100 in PBS at room temperature for at least 30 minutes. Cells were then incubated with primary antibody (Anti-MYO7A (Abcam, ab150386): 1:500) in blocking solution at 4° C. overnight. Following antibody incubation, cells were washed three times with 1×PBS for five minutes and then incubated with secondary antibody (Goat anti-rabbit 594 at 1:500) in blocking solution at room temperature for two hours in the dark. Cells were then washed three times with PBS for five minutes and stained with DAPI for 15 minutes. Following DAPI staining, cells were washed with PBS and imaged. Expression was observed for all constructs, and the pattern of expression between GFP and MYO7A was consistent across all constructs. The expression constructs UID 1484 induced the highest expression of MYO7A as assessed by GFP expression, as shown in FIG. 6.

### Example 4. Expression of DNA Vectors Containing S/MAR and Truncated S/MAR

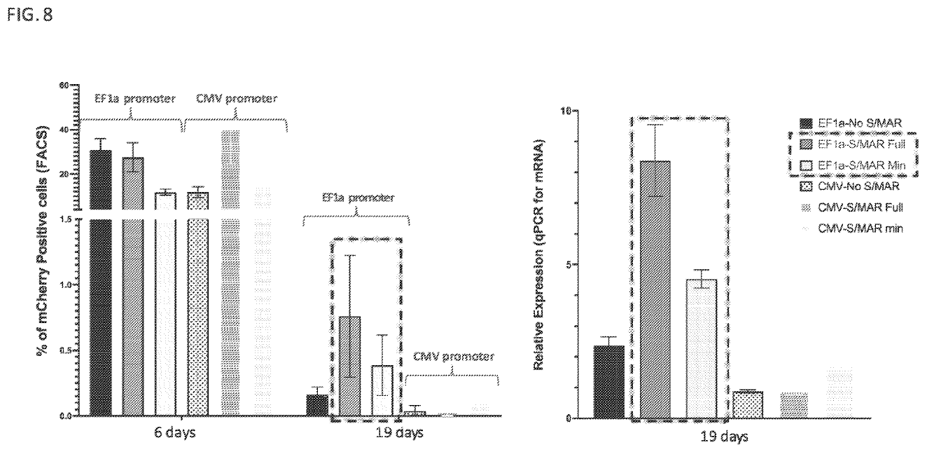

An interferon-β (IFN-β) Scaffold Matrix Attachment Region (S/MAR; SEQ ID NO: 10) and truncated variant thereof (S/MAR min; SEQ ID NO: 11) were included in select plasmid expression constructs: UID 1685, 1547, 1548, 1549, 1550, 1557, 1493, 1495, 1497, and 1484. The expression efficiency of S/MAR-containing constructs is demonstrated in FIGS. 3-8. In vitro assays assessing the impact of the S/MAR were performed using K562 cells, which are rapidly dividing cells. Inclusion of S/MAR sequences in the expression construct with an EF1 A promoter (SEQ ID NO: 15) resulted in expression of both mCherry (FACS) and relative mRNA (qPCR) after 19 days, while the CMV promoter constructs lost gene expression FIG. 8.

### Example 5. In Vivo Expression of a Synthetic Circular DNA Vector Containing S/MAR and Truncated S/MAR

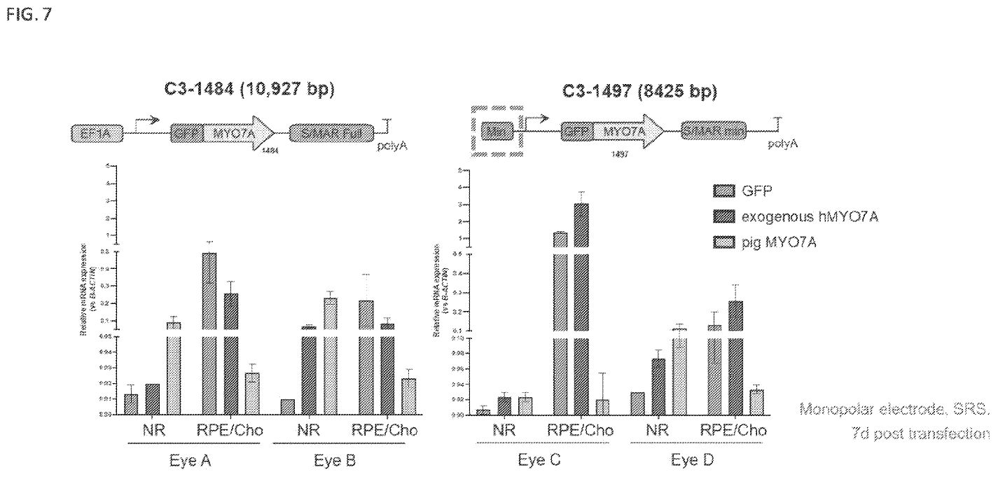

C³DNA vectors 1484 (C³-1484; 10,927 bp) and 1497 (C3-1497; 8,325 bp)) were selected for in vivo expression experiments. C³-1484 and C-1497 were formulated in a solution at a concentration of 1.5 mg/mL. Naked vector was administered to Gottingen Minipigs by injecting two blebs (75 μL each) into the subretinal space (225 ug DNA; 2.53×10¹³ vector copies per eye). After injection, a monopolar needle electrode was placed within each subretinal bleb, and eight 20-ms electrical pulses were transmitted at 20V. Relative mRNA expression of GFP, exogenous human MYO7A (hMYO7A) and pig MYO7A was measured in the RPF/choroid and NR layers of the eye seven days after the initial transfection. Robust detection of exogenous sequences (both GFP and MYO7A) was observed in RPE/choroid tissues is both C³-1484 and C-1497-treated animals, indicating robust expression in RPE/choroid, using endogenous pig MYO7A as a control (FIG. 7).

### Example 6. Identification of Regulatory Sequences for Long-Term Episomal Persistence

Screening for novel, native regulatory sequences was performed DNA was purified from the nuclear matrix of ARPE-19 or iRPE cells and a library was generated by end-repair and ligation of DNA into barcoded plasmids. To obtain a manageable range of sizes (in bp) for library construction, the nuclear matrix isolated from iRPE cells was sheared prior to library construction. The library was then delivered to cells, e.g., iRPE cells, and fluorescent reporters were used for the long-term assessment of gene expression. Positive cells were selected and the DNA was sequenced to identify relevant regulatory sequences that confer long-term durability of gene expression and increased episomal persistence. This method revealed a sequence within intron 6 of ABCA4 (nucleotides 3158-4822 of ABCA4 intron 6; intron 6 RE (i6RE)) as a potential regulatory element.

### Example 7. In Vitro Expression of DNA Vectors Containing ABCA4 Intron 6 RE

Plasmid DNA vectors encoding human ABCA4 driven by various promoters and including an ABCA4 intron 6 RE (i6RE) of SEQ ID NO: 13 were produced as described in Example 1. The ABCA4 i6RE was modified form the region of ABCA4 intron 6 from which it was derived to allow for BsaI restriction digest by deleting a G at position 3530 of native human ABCA4 intron 6 to remove a BsaI recognition site, as described above.

Four promoters were tested: Exon_Intron1_Large (SEQ ID NO: 7, within p-1551 (SEQ ID NO: 22)), Exon_Intron1_Short (SEQ ID NO: 6, within p-1552 (SEQ ID NO: 23)), Promoter_Large (SEQ ID NO: 8, within p-1553 (SEQ ID NO: 24)), and Promoter_Short (SEQ ID NO: 9, within p-1554 (SEQ ID NO: 25)).

In vitro expression was tested in the experiments described in Example 2, and results are shown in FIGS. 3 and 4. Observed expression by copy number of each plasmid vector containing i6RE (p-1551, p-1552, p-1553, and p-1554) were greater than their respective controls containing truncated S/MAR (p-1547, p-1548, p-1549, and p-1550)(FIG. 3).

### Example 8. In Vivo Expression of a DNA Vector Containing ABCA4 Intron 6 RE

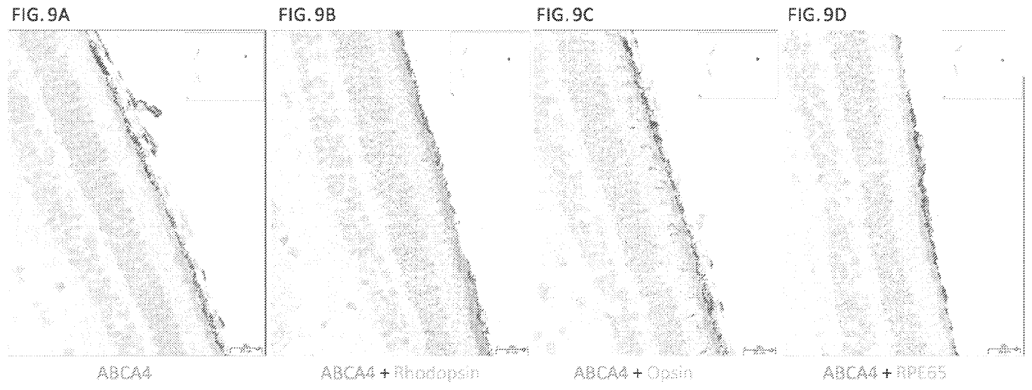

A synthetic C³ DNA vector encoding human ABCA4 driven by a CAG promoter and including an ABCA4 intron 6 RE of SEQ ID NO: 13 (C³-ABCA4-i6RE; SEQ ID NO: 26) was produced using Phi29 polymerase-mediated rolling circle amplification in the cell-free process described in Example 1. C³-ABCA4-i6RE was formulated in a solution at a concentration of 1.5 mg/mL. Naked C³-ABCA4-i6RE was administered by injecting two blebs (75 μL and 40 μL) into the subretinal space of Gottingen Minipig (172.5 ug DNA: 1.94×10¹³ vector copies per eye). After injection, a monopolar needle electrode was placed within each subretinal bleb, and eight 20-ms electrical pulses were transmitted at 20V. Euthanization was performed after six days, and tissues were dissected to collect the retina and RPE and choroid for staining. Surprisingly, robust staining for human ABCA4 protein was observed in the photoreceptor outer segment layer (FIGS. 9A-9D). Human ABCA4 protein expression was co-localized with rhodopsin and did not co-localize with RPE6S, indicating that the ABCA4 transgene was preferentially expressed in photoreceptors. Together, these results indicate that delivery of C³-ABCA4-i6RE to the retina unexpectedly led to preferential expression of human ABCA4 protein in the desired cell type (photoreceptors) at a desired subcellular location within those cells (outer segments).

SEQUENCE TABLE

| SEQ ID NO: | |
|---|---|
| 1 | [symbol] |
| ABCA4 coding | |
| sequence | |
| 2 | [symbol] |
| MYO7A coding | |
| sequence | |
| 3 | [symbol] |
| MYO7A Promoter | |
| HS1/2_Intron1 | |
| 4 | [symbol] |
| MYO7A Promoter | |
| HS1-3 | |
| 5 | [symbol] |
| MYO7A Promotex | |
| Min | |
| 6 | [symbol] |
| ASCA4 Promoter | |
| Exon_Intron1_Short | |
| 7 | [symbol] |
| ABCA4 Promoter | |
| Exon_Intron1_large | |
| 8 | [symbol] |
| ABCA4 Promoter_ | |
| Large | |
| 9 | [symbol] |
| ABCA4 Promoter_ | |
| Short | |
| 10 | [symbol] |
| INFγ S/MAR | |
| 11 | [symbol] |
| S/MAR | |
| 12 | [symbol] |
| cHS4 | |
| 13 | [symbol] |
| ABCA4 intron 6 | |
| (portion) | |
| 14 | [symbol] |
| CAG Promoter | |
| 15 | [symbol] |
| EF1A Promoter | |
| 16 | CCTAGCCTTTAAGGGGCTCTA |
| Forward Primer | |
| exon 7 of R81 | |
| 17 | TGACCTGTGTGAAAAGATCAGGG |
| Reverse Primer | |
| exon 7 of R81 | |
| 18 | TGGGAGTTAGACCCGGCGAGTG |
| Forward Primer | |
| (ABCA4) | |
| 19 | TGTAGCATCTCCGCTTGTCACT |
| Reverse Primer | |
| (ABCA4) | |
| 20 | CAGTCTTCTGGGTGGCAGTG |
| Forward Primer | |
| (GAPDH) | |
| 21 | AACCATGAGAAGTATGACAACAGC |
| Reverse Primer | |
| (GAPDH) | |
| 22 | [symbol] |
| p1551 | |
| 23 | [symbol] |
| p1552 | |
| 24 | [symbol] |
| p1553 | |
| 25 | [symbol] |
| p1554 | |
| 26 | [symbol] |
| C³-ABCA4-16RE | |
| 27 | [symbol] |
| Expression | |
| construct with | |
| CAG promoter, | |
| ABCA4 coding | |
| sequence, and | |
| 3/MAR regulatory | |
| element | |
| 28 | [symbol] |
| ABCA4 construct | |
| with cHS4 | |
| 29 | [symbol] |
| ABCA4 intron 6 | |
| 30 | [symbol] |
| Expression | |
| construct with | |
| CAG promoter, | |
| ABCA4 coding | |
| sequence, and | |
| S/MAR regulatory | |
| element | |
| 31 | [symbol] |
| MYO7A amino | |
| acid sequence | |
| 32 | [symbol] |
| ARCA4 amino | |
| acid sequence | |
| 33 | [symbol] |
| [symbol] | |
| replication | |
| protein | |
| 34 | AGGGCGCTGTTATCTGATAAGGCTTATCTGGTCTCATTTT |
| [symbol] origin of | |
| replication | |
| 35 | AGGGCGCTGTTATCTGATAAGGCTTATCTGGTCTCA |
| Col52-99 origin of | |
| replication | |
| 36 | GCGCTGTTATCTGATAAGGCTTATCTGGTCTCA |
| [symbol] origin of | |
| replication | |
| 37 | TGTTATCTGATAAGGCTTATCTGGTCT |
| [symbol] origin of | |
| replication | |
| 38 | TGTTATCTGATAAGGCTTATCTGGTCTC |
| [symbol] origin of | |
| replication | |
| 39 | TGTTATCTGATAAGGCTTATCTGGTCTCA |
| [symbol] origin of | |
| replication | |
| 40 | CTGTTATCTGATAAGGCTTATCTGGTCTCA |
| [symbol] origin of | |
| replication | |
| 41 | GCTGTTATCTGATAAGGCTTATCTGGTCTCA |
| [symbol] origin of | |
| replication | |
| 42 | CGCTGTTATCTGATAAGGCTTATCTGGTCTCA |
| [symbol] origin of | |
| replication | |
| 43 | X1X2X3X4X5TGTTATCTGATAAGGCTTATCTGGTCTX6X7 |
| [symbol] origin of | |
| replication | |
| 44 | [symbol] |
| Codon optimized | |
| ABCA4 coding | |
| sequence | |
| 45 | [symbol] |
| Codon | |
| optimized | |
| MyO7A coding | |
| sequence | |

[symbol]

indicates data missing or illegible when filed

What is claimed is:

1.-21. (canceled)

22. A nucleic acid vector comprising:
(a) a promoter comprising a native ABCA4 promoter or functional variant thereof;
(b) a cDNA sequence encoding ABCA4; and
(c) a regulatory element.

23. (canceled)

24. The nucleic acid vector of claim 22, wherein the regulatory element comprises a scaffold/matrix attachment region (S/MAR) sequence.

25. (canceled)

26. The nucleic acid vector of claim 24, wherein the S/MAR comprises the nucleic acid sequence of SEQ ID NO: 10 or 11, or a functional variant thereof.

27. The nucleic acid vector of claim 22, wherein the regulatory element comprises a sequence derived from ABCA4 intron 6.

28.-34. (canceled)

35. The nucleic acid vector of claim 22, wherein the nucleic acid vector is a circular DNA vector selected from plasmid DNA vector, a minicircle DNA vector, a synthetic circular DNA vector, a closed-ended DNA vector, a doggybone DNA vector, or a ministring DNA vector.

36. The nucleic acid vector of claim 35, wherein the nucleic acid vector lacks a drug resistance gene.

37. The nucleic acid vector of claim 36, wherein the nucleic acid vector comprises a truncated ColE2-P9 replication origin.

38.-62. (canceled)

63. A nucleic acid vector comprising:
(a) a promoter comprising any one of SEQ ID NOs: 3-5; and
(b) a cDNA sequence encoding MYO7A.

64. The nucleic acid vector of claim 63, further comprising a regulatory element that enhances the expression and/or persistence of MYO7A.

65. The nucleic acid vector of claim 64, wherein the regulatory element comprises a scaffold/matrix attachment region (S/MAR) sequence.

66. (canceled)

67. The nucleic acid vector of claim 65, wherein the S/MAR comprises the nucleic acid sequence of SEQ ID NO: 10 or 11.

68.-71. (canceled)

72. The nucleic acid vector of claim 64, wherein the regulatory element comprises the nucleic acid sequence of SEQ ID NO: 13 or a functional variant thereof.

73.-75. (canceled)

76. The nucleic acid vector of claim 63, wherein the nucleic acid vector is a non-integrating DNA vector.

77. The nucleic acid vector of claim 76, wherein the nucleic acid vector is a circular DNA vector selected from a plasmid DNA vector, a minicircle DNA vector, a synthetic circular DNA vector, a closed-ended DNA vector, a doggybone DNA vector, or a ministring DNA vector.

78. The nucleic acid vector of claim 77, wherein the nucleic acid vector lacks a drug resistance gene.

79.-124. (canceled)

125. A method of treating an ocular disorder in a subject, the method comprising administering to the subject the nucleic acid vector of claim 22 or a pharmaceutical composition comprising a therapeutically effective amount of the nucleic acid vector of claim 22 and a pharmaceutically acceptable carrier.

126.-127. (canceled)

128. The method of claim 125, wherein the method expresses ABCA4 in the eye of a subject.

129.-131. (canceled)

132. The method of claim 125, wherein the method expresses MYO7A in the eye of a subject.

133.-134. (canceled)

135. The method of claim 125, wherein the nucleic acid vector is a circular DNA vector delivered by in vivo electroporation.

136. The method of claim 135, wherein the in vivo electroporation comprises:
(a) contacting an electrode to an interior region of an eye of the subject, wherein an extracellular space in the retina of the eye comprises the nucleic acid vector; and
(b) while the electrode is contacting the interior region of the eye, transmitting one or more pulses of electrical energy through the electrode at conditions suitable for electrotransfer of the nucleic acid vector into a target retinal cell.

137.-148. (canceled)


SEQUENCE LISTING (45 sequences)

SEQ ID NO: 1 - DNA, 6822 bp, synthetic construct
  source 1..6822: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 2 - DNA, 6645 bp, synthetic construct
  source 1..6645: mol_type = other DNA
gtcattctgcagcagggcgaccacgtgtggatggatctgagactgggccaagagttcgacgtgccaatcggcgccgtggtcaagctgtgtgattctggccaggtgcaggtcgtggacgacgaggataatgagcactggatcagccctcagaacgccacacacatcaagcctatgcaccccacatctgtgcacggcgtggaagatatgatccggctgggcgatctgaacgaggccggcatcctgagaaacctgctgatcagataccgggaccacctgatctacacctacaccggctctatcctggtggccgtgaatccctaccagctcctgagcatctacagccccgagcacatccggcagtacaccaacaagaaaatcggcgagatgcctcctcacatcttcgccattgccgacaactgctacttcaacatgaagcggaacagccgggaccagtgctgcatcatctctggcgaatctggcgccggaaagaccgagagcacaaagctgatcctgcagttcctggccgccatcagcggacagcactcttggattgagcagcaggtcctggaagccacacctattctggaagccttcggcaacgccaagaccatccggaacgacaacagcagcagattcggcaagtacatcgacatccacttcaacaagagaggcgccattgagggcgccaagatcgagcagtacctgctggaaaagtccagagtgtgcagacaggccctggacgagagaaactaccacgtgttctactgcatgctggaaggcatgagcgaggaccagaagaagaagctcggactcggccaggccagcgactacaattatctggccatgggcaactgcatcacatgcgagggcagagtggacagccaagagtacgccaacatccgcagcgccatgaaggtgctgatgttcaccgacaccgagaactgggagatcagcaaactgctggccgctatcctgcatctgggcaacctgcagtacgaggccagaaccttcgagaacctggatgcctgcgaggtgctgttctctcctagcctggctacagccgcctctctgctggaagtgaaccctcctgatctgatgagctgcctgaccagcagaaccctgatcaccagaggcgagacagtgtctacccctctgagcagagaacaggctctggatgtgcgggacgccttcgtgaagggcatctacggcagactgttcgtgtggatcgtggacaagatcaacgccgccatctacaagcctccaagccaggacgtgaagaacagcagaagatccatcggcctgctggacatcttcggcttcgagaatttcgccgtgaacagcttcgagcagctctgcatcaacttcgccaacgagcacctccagcagttcttcgtgcggcacgtgttcaagctggaacaagaggaatacgacctggaatccatcgactggctgcacatcgagttcaccgataaccaggacgccctggacatgatcgccaacaagcccatgaacatcatcagcctgatcgacgaggaaagcaagttccccaagggcaccgataccaccatgctgcacaagctgaacagccagcacaaactgaatgccaactacatcccgcctaagaacaaccacgagacacagttcggcatcaaccacttcgccggcatcgtgtactacgaaacccagggctttctggaaaagaaccgggacaccctgcacggcgacatcattcagctcgtgcacagcagccggaacaagttcatcaagcagatcttccaggccgacgtcgccatgggagccgagacaagaaagagaagccccacactgagcagccagttcaagcggagtctggaactgctgatgagaaccctgggagcctgccagcctttctttgtgcggtgcatcaagcccaacgagttcaagaaacccatgctgttcgaccggcacctgtgtgtgcggcagctcagatacagcggcatgatggaaaccatcaggattcggagagccggctatcccatccggtacagcttcgtggagttcgtcgagcggtacagagtgctgctgcctggcgtgaagcctgcctacaaacagggcgatctcagaggcacctgtcagagaatggccgaagccgtgctgggcacccatgacgattggcagatcggaaagacaaagatcttcctgaaggaccaccacgacatgctgctcgaggtggaaagagacaaggccatcaccgacagagtgatcctgctccagaaagtgatccggggcttcaaggacagaagcaatttcctgaagctgaagaatgccgccactctgatccagagacactggcggggacacaactgccggaagaactacggcctgatgaggctgggcttcctgagactgcaggccctgcacagaagcagaaagctgcaccagcagtacagactggcccggcagcggatcatccagtttcaagccagatgtcgggcctacctcgtgcgcaaggccttcagacatagactgtgggccgtgctgaccgtgcaggcctatgccagaggaatgattgcccgcagactgcaccagagactgagagccgagtatctgtggcggctggaagccgagaaaatgcggctggccgaggaagagaagctgcggaaagagatgagcgccaagaaggccaaagaagaggccgagcggaagcaccaagagagactggctcaactggccagagaggacgccgagagagagctgaaagagaaagaggccgccagacggaagaaagaactcctggaacagatggaacgggccagacacgagcccgtgaaccacagcgatatggtggataagatgttcggcttcctgggcacctctggcggactgcctggacaagaaggacaggcccctagcggctttgaggacctggaacgtgggagaagagaaatggtggaagaggatctggacgccgctctgcctctgcctgacgaggatgaagaagatctgagcgagtacaagttcgccaagtttgccgccacctactttcagggcaccaccacacacagctacaccagaaggcctctgaagcagcccctgctgtaccacgatgatgagggcgatcaactggcagccctggccgtgtggattaccatcctcagattcatgggcgacctgcctgagcctaagtaccacaccgccatgtctgacggctccgagaagatccccgtgatgaccaagatctacgagactctgggcaagaaaacctacaagcgcgagctgcaggctctccaaggcgaaggcgaagctcaactgcctgagggccagaaaaagtcctctgtgcgccacaaactggtgcacctgacactgaagaagaaaagcaagctgacagaggaagtgaccaagcggctgcacgatggcgagtctacagtgcagggcaacagcatgctcgaggacagacccaccagcaacctggaaaaactgcacttcatcatcggcaacggaatcctgcggcctgctctgagggatgagatctactgccagatctccaagcagctcacacacaaccccagcaagagcagctacgccagaggctggattctggtgtctctgtgcgtgggctgtttcgccccaagcgagaagttcgtgaagtacctgaggaacttcatccacggcggacctccaggctacgccccttactgtgaagagaggctgagaaggacctttgtgaacggcacccggacacagcctccatcctggctggaactccaggccaccaagagcaaaaagcccatcatgctgcccgtgacctttatggatggcaccacaaagaccctgctgaccgatagcgccaccaccgccaaagagctgtgtaacgccctggctgacaagattagcctgaaggatagattcggcttcagcctgtacattgccctgttcgacaaggtgtccagcctcggctctggctctgaccatgtgatggatgccatcagccagtgcgagcagtatgccaaagaacagggcgcccaagagaggaacgctccttggcggctgttctttcggaaagaggtgttcaccccttggcacagccccagcgaagataacgtggccaccaatctgatctaccagcaggttgtgcggggcgtgaagttcggcgagtacagatgcgaaaaagaggacgatctggccgagctggcctctcagcagtactttgtggactacggcagcgagatgatcctggaacggctgctgaatctggtgcccacctacattcccgatagagagatcaccccactgaaaaccctcgagaagtgggcccagctcgccattgctgcccacaagaaaggcatctatgcccagcggagaacagacgcccagaaagtcaaagaggatgtcgttagctacgcccggttcaagtggcctctgctgtttagcagattctacgaggcctacaagttcagcggccccagtctgcccaagaacgatgtgatcgtggctgtgaactggaccggcgtgtacttcgtggatgagcaagaacaagtgctgcttgagctgagcttccccgagatcatggccgtgtccagctccagagaatgcagagtgtggctgagcctgggctgtagcgatctgggatgtgccgctcctcattctggatgggctggactgacaccagccggaccttgtagcccttgttggtcttgcagaggggccaagacaacagcccctagctttaccctggccaccattaagggcgacgagtacaccttcaccagcagcaacgccgaggacatcagagatctggtcgtgaccttcctggaaggcctgcggaagcggagcaaatatgtggtggccctgcaggacaaccccaatcctgctggcgaggaatccggctttctgagctttgccaaaggcgacctgatcatcctggaccacgacaccggcgagcaagtgatgaatagcggctgggccaacggcatcaatgagcggacaaagcagcggggcgacttccctacagatagcgtgtacgtgatgcccaccgtgaccatgcctccaagggaaatcgtggccctggtcaccatgacacccgaccagagacaggatgttgtgcggctgctgcagctcaggacagccgaaccagaagtgcgggccaagccttacacactggaagagttcagctacgactacttccggcctcctccaaagcacaccctgtctagagtgatggtgtccaaggccagaggcaaggataggctgtggtcccacacaagagagcccctgaaacaggcactgctgaaaaagctgctgggcagcgaggaactgagccaagaagcctgtctggcctttatcgcagtgctgaagtacatgggcgattacccctccaagcggaccagatccgtgaacgaactgaccgaccagattttcgagggcccactgaaggccgagcctctgaaagatgaggcctacgtgcagattctgaaacagctcaccgacaaccacatccgctacagcgaggaacgcggatgggaactgctgtggctgtgtaccggactgttcccacctagcaacattctgctgccccacgtgcagcggtttctgcagtctagaaagcactgccctctggccatcgattgcctgcagaggctgcaaaaggccctgagaaatggctcccggaagtaccctcctcacctggtggaagtggaagccatccagcacaagaccacacagatctttcacaaggtctacttccccgacgacacagacgaggcctttgaggtggaatcctctaccaaggccaaggacttctgccagaatatcgccaccaggctgctgctgaagtccagcgaaggctttagcctgtttgtgaagatcgccgacaaagtgctgagcgtgcccgagaacgacttctttttcgattttgtgcgccatctgaccgactggattaagaaggctagacccatcaaggatggcatcgtgcccagcctgacctatcaggtgttctttatgaagaagctgtggacgaccaccgtgcctggcaaggatcctatggccgacagcatcttccactactaccaagagctgcccaagtacctgcggggctaccacaagtgtaccagagaagaggtcctgcagctcggagccctgatctatagagtgaagtttgaagaggacaagagctacttccctagcatccccaagctgctgcgcgaactggttccccaggatctgatccggcaagtgtcccctgatgactggaagcggtctatcgtggcctactttaacaagcacgccggcaagagtaaagaggaagccaagctggcctttctgaagctcatctttaagtggcctaccttcggctccgccttcttcgaagtgaagcagaccaccgagcctaacttccctgagattctgctgatcgccatcaacaaatacggcgtgtccctgatcgatcccaagacaaaggacatcctgacaacacaccccttcaccaaaatcagcaactggtccagcggcaacacctacttccacatcaccatcggcaatctcgtgcggggctctaagctgctgtgtgaaaccagcctgggatacaagatggacgacctgctgacaagctacatctcccagatgctgaccgccatgagcaaacagagaggctctcggagcggcaagtga

SEQ ID NO: 3 - DNA, 3763 bp, synthetic construct
  source 1..3763: mol_type = other DNA
cctgtcccccataacctgcccacctctctgccccattcccacagctcactgcattttgtccctgctgccagtcgtgtgccatgggctctcctgtgctcccccgggctctcatttccccatccgtgtagggctgacgccactggccccgcccagcctgggaggatggagaggggggacttgaggagtggccagtactgggtttcggcagcaggtgcaaatcccaggtggggactgtgttccagggactcctggactcctgttaggccagcaggctaaggggcagacggcatcctggggtcttcctgggacagggtcgtcgggtctgaggggacccaggccctagcagaagctgactcctgtcacggtcacttcccagcggtgctcagcccgcctgacggaagcagctgggcagtggggcctgtgaccagcaggcggcgctggcgagctatagcgcccctttgggcccccaccccaccttctgggcagcttccctgcagacaccccagttatgggggctagggacccaaaagagacatccttctgccacccagagctgccctggcgaggtgcactatggggccgccgacagctgcgtggctgccgagggcggaaaggagaaactgtcatgtcccgatagggccgcgcgagctctccatcctcgacaacgctaataacaaagacgtgtgctcctctttgcttggttccccccactcctttaaatcacagatttcacttcagtttatctgtgtcgctgtcacacgtggggtggctcccagtcagctggtttggcaaagtttctggatgattacggaataacatgtgtccccaacccgcagagcaggttgtgggggcaatgttgcattgaccagcgtcagagaacacacatcagaggcaagggtgggtgtgcaggagggagaaggcgcagaaggcagggctttagctcagcactctccctcctgccatgctctgcctgaccgttccctctctgagtcccaaacagccaggtagaggaggaagaaatggggctgagagcccagcacatcagtgattaagtcaggatcaggtgcggtttcctgctcaggtgctgagacagcaggcggtgtcctgcaaacaacaggaggcacctgaagctagcctggggggcccacgcccaggtgcggtgcattcagcagcacagccagagacagaccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagcagtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccaggtaaggatgggctgcccactgtcctgggcattgggaggggtttggatgtggaggagtcatggacttgagctacctctagagcctctgccccacagccacttgctcctgggactgggcttcctgccacccttgagggctcagccaccacagccactgaatgaaactgtcccgagcctgggaagatggatgtgtgtcccctggaggagggaagagacaaggagcatgttgtccatcgaatcttctctgagctggggctggggttagtggcatcctggggccaggggaatagacatgctgtggtggcagagagaagagtccgttctctctgtctcctttgctttctctctgacactctttatctccgtttttggataagtcacttccttcctctatgccccaaatatcccatctgtgaaatgggagtatgaagccccaacagccagggttgtagtggggaagaggtaaaatcaggtatagacatagaaatacaaatacagtctatgccccctgttgtcagttggaaaagaaattaacttgaaggtggtctagttctcatttttagaaatgaaatgtctgtctggtcattttaaaatgtggcccttaaatttcacgccctcaccactctcccccatcccttggagccccatgtctctagtgaaagcactggctctgcccccagccctcatggctcatgctggcatagggcgcctgctccacagcctgggcaccatcttcagacaagtgcccggtggcaactgcctgctggccctgttgaatccacatctccaccaggcatccagactagttcagctctctggaaggactgtgggtttgctgtgtcccagagctccagggcaggggtcagggctcggatgtcgggcagtgtcatgggcagaggatcgaatgccccggcggctctgaatgggcccttgtgaaaaattgatgcgcattctaggagacaggttgggagccagaggggcctcataccagggtctgtaggctggggctgccttttaagctccttcctgaggccgtctctgggtctggccctgtgctggacaaggctggagacaaggcaatgtctcagaccctctcccattggccacatcctgccctggatcaactcgccaactttgggggcagaggtgggactgacccttaccctgacaacataatgcatatagtcaaaatgggataaaggggaatatagaggctcttggcagcttgggagtggtcagggaaggcttcctggaggaggtatcatctgaactgagccatgaaccataagtggaaattcactagtcaaaatttcaggtagaagggccagtgtgtgaaggccaggagatggcaagagctggcgtatttcaggaacagtgagtcactgaggatgtccaagtataagggtaggaaagggagtgagcagtgagagaaaagaccgaggcatcagcaggggccagattgtgctgggcctagcggggcgggcccgggcccgggcccaggcccaggtgcggtgcattcagcagcacagccagagacagaccccaatgaccctgcctccccgtcagcagccagtgctctgcacagagccatcctgagggcagtgggtgctcttgagaggtttcaggcagggtgtgctgtgagcaggtcatgcccagcccttgaccttctgctcagtcaggcttgtccttgtcacccacattcctggggcagtccctaagctgagtgccggagattaagtcctagtcctaaatttgctctggctagctgtgtgaccctgggcaagtcttggtccctctctgggcccctttgccgtaggtccctggtggggccagacttgctactttctaggagccctttgggaatctctgaatgacagtggctgagagaagaattcagctgctctgggcagtggtgctggtgacagtggctgaggctcaggtcacacaggctgggcagtggtcagagggagagaagccaaggagggttcccttgagggaggaggagctggggctttgggaggagcccaggtgaccccagccaggctcaaggcttccagggctggcctgcccagaagcatgacatgctctctctccctgcagaactgtgcctggcccagtgggcagcaggagctcctgacttgggacc

SEQ ID NO: 4 - DNA, 1266 bp, synthetic construct
  source 1..1266: mol_type = other DNA
gggcggaaaggagaaactgtcatgtcccgatagggccgcgcgagctctccatcctcgacaacgctaataacaaagacgtgtgctcctctttgcttggttccccccactcctttaaatcacagatttcacttcagtttatctgtgtcgctgtcacacgtggggtggctcccagtcagctggtttggcaaagtttctggatgattacggaataacatgtgtccccaacccgcagagcaggttgtgggggcaatgttgcattgaccagcgtcagagaacacacatcagaggcaagggtgggtgtgcaggagggagaaggcgcagaaggcagggctttagctcagcactctccctcctgccatgctctgcctgaccgttccctctctgagtcccaaacagccaggtagaggaggaagaaatggggctgagagcccagcacatcagtgattaagtcaggatcaggtgcggtttcctgctcaggtgctgagacagcaggcggtgtcctgcaaacaacaggaggcacctgaagctagcctggggggcccacgcccaggtgcggtgcattcagcagcacagccagagacagaccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagcagtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgccgagctgagtcctccagccccccttcctactgagggccacagtggcctctgaccctcagcccttccccgtgatcagtctgaggctctaatgagctcctggcctccagagcaggagccaggaatgtgggccctgagccaggtgggcctggcagcaggccgtagggcttgtggggctgtgcattatgggggcttggccagcagcttctcgctctccagggtcaccagaggccaatggctcagccttgcctggccaccctgaggctgcacggaccagcatgtttgcatatgagtgcgtgcgcgtgtgtgtgtctgtgtgtgtgagtgagaggcagg

SEQ ID NO: 5 - DNA, 348 bp, synthetic construct
  source 1..348: mol_type = other DNA
ccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagcagtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccg

SEQ ID NO: 6 - DNA, 923 bp, synthetic construct
  source 1..923: mol_type = other DNA
agggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggcctta

SEQ ID NO: 7 - DNA, 1810 bp, synthetic construct
  source 1..1810: mol_type = other DNA
caagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggcctta

SEQ ID NO: 8 - DNA, 1382 bp, synthetic construct
  source 1..1382: mol_type = other DNA
caagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattag

SEQ ID NO: 9 - DNA, 495 bp, synthetic construct
  source 1..495: mol_type = other DNA
agggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattag

SEQ ID NO: 10 - DNA, 2206 bp, synthetic construct
  source 1..2206: mol_type = other DNA
gaattcagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgattgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagtggaatgatttaggaagtattccctctgcttctgtcttctgaaagagattgtagaaagttgatacaatttttttttctttaaatatcttgatagaattc

SEQ ID NO: 11 - DNA, 540 bp, synthetic construct
  source 1..540: mol_type = other DNA
gcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacct

SEQ ID NO: 12 - DNA, 2062 bp, synthetic construct
  source 1..2062: mol_type = other DNA
acacagcctgaagcccagcggctgtgctatgggctggggacggctctgtgctcagcatccttcaatccacggcggctccgctgcacctcctctgcaaaacgcctccaggaaaccactcggccgagtgccctctcccggagccttacttagagcctcaggaaaaggaagaaagtgccgaaaggttgaagaaaagaagcaggctttcctggaaggtcctggaagggggcgtccgcgggagctcacggggacagcccccccccaaagcccccagggctgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagctttaggctgaaagagagatttagaatgacagaatcatagaacggcctgggttgcaaggagcacagtgctcatccagatccaaccccctgctatgtgcagggtcatcaaccagcagcccagcgcgtcagagccacatccagcctggccttgaatgcctgcctgcagggatggggcatccacagcctccttgggcaacctgttcagtgcgtcaccaccctctggggaaaaactgcctcctcatatccaacccaaacctcccctgtctcagtgtaaagccattcccccttgtcctatcaagggggagtttgctgtgacattgttggtctggggtgacacatgtttgccaattcagtgctcacggagaggcagatcttgggataaggaagtgcaggacagcatggacgtggacatgcaggtgttgaggctctggacactccaagtcacagcgttcagaacagccttaaggtcaagaagataggatagaaggacaaagagcaagttaaaacccagcatggagaggagcacaaaaaggccacagacactgctggtccctgtgtctgagcctgcatgtttgatggtgtctggatgcaagcagaaggggtggatgagcttgcctggagagatacaggctgggtcgtaggactgggacaggcagctggagaattgccatgtagatgttcatacaatcgtcaaatcatgaaggctggaaaagccctccaagatccccaagaccaaccccaacccacccaccgtgccactggccatgtccctcagtgccacatccccacagttcttcatcacctccagggacggtgaccccccacctccgtgggcagctgtgccactgcagcaccgctctttggagaaggtaaatcttgctaaatccagcccgaccctcccctgggcacaacgttaaggccattatctctcatcctactccaggacggagtcagttgaggatgggggaatattgctgtgggtggcagatggccacatcccatgtccctcagtgccacatccccacagttcttcatcacctccagggacggtgaccccccacctccgtgggcagctgtgccactgcagcaccgctctttggagaaggtaaatcttgctaaatccagcccgaccctcccctgggcacaacgttaaggccattatctctcatcctactccaggacggagtcagttgaggatgggggaatattgctgtgggtggcagatggccacatcctggtcaaaggctataggcagaacgaagacacattcaatgtctgtgaggctgtgagtgaaaagcatttggaagaggcaggtgctaagctgatctctctggctccggccacagcatcctcagtgtggttgggattgcagatgtggacaaggccaggactgccacagctggcatagcgaggaggaggtacatggcacgtgcaggctctgctctgccttaatgacaacagggaacatccagttccccagcagcaccaggaggtacatggagggggcagctgagtggttttccatgatggaaaagccaccaagaagaatatcagcatccttggtctggctgctgttggtccaaggtg

SEQ ID NO: 13 - DNA, 1664 bp, synthetic construct
  source 1..1664: mol_type = other DNA
agaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggagg

SEQ ID NO: 14 - DNA, 1681 bp, synthetic construct
  source 1..1681: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaa

SEQ ID NO: 15 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 16 - DNA, 21 bp, synthetic construct
  source 1..21: mol_type = other DNA
cctagcctttaaggggctcta

SEQ ID NO: 17 - DNA, 23 bp, synthetic construct
  source 1..23: mol_type = other DNA
tgacctctgtgaaaagatcaggg

SEQ ID NO: 18 - DNA, 22 bp, synthetic construct
  source 1..22: mol_type = other DNA
tgggagttagacccggcgagtg

SEQ ID NO: 19 - DNA, 22 bp, synthetic construct
  source 1..22: mol_type = other DNA
tgtagcatctccgcttgtcact

SEQ ID NO: 20 - DNA, 20 bp, synthetic construct
  source 1..20: mol_type = other DNA
cagtcttctgggtggcagtg

SEQ ID NO: 21 - DNA, 24 bp, synthetic construct
  source 1..24: mol_type = other DNA
aaccatgagaagtatgacaacagc

SEQ ID NO: 22 - DNA, 12495 bp, synthetic construct
  source 1..12495: mol_type = other DNA
ttcggtatcacaggagaatttcagggagcaagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggccttacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 23 - DNA, 11608 bp, synthetic construct
  source 1..11608: mol_type = other DNA
ttcggtatcacaggagaatttcagggagagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggccttacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 24 - DNA, 12067 bp, synthetic construct
  source 1..12067: mol_type = other DNA
ttcggtatcacaggagaatttcagggagcaagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 25 - DNA, 11180 bp, synthetic construct
  source 1..11180: mol_type = other DNA
ttcggtatcacaggagaatttcagggagagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 26 - DNA, 10301 bp, synthetic construct
  source 1..10301: mol_type = other DNA
ttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggactaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctactaaaaactc

SEQ ID NO: 27 - DNA, 10843 bp, synthetic construct
  source 1..10843: mol_type = other DNA
aaaaactcttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggactaagcttgaattcagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgattgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagtggaatgatttaggaagtattccctctgcttctgtcttctgaaagagattgtagaaagttgatacaatttttttttctttaaatatcttgatagaattcataaaataaaatatctttattttcattacatctgtgtgttggttttttgtgtgcgctact

SEQ ID NO: 28 - DNA, 10699 bp, synthetic construct
  source 1..10699: mol_type = other DNA
aaaaactcttcgacacagcctgaagcccagcggctgtgctatgggctggggacggctctgtgctcagcatccttcaatccacggcggctccgctgcacctcctctgcaaaacgcctccaggaaaccactcggccgagtgccctctcccggagccttacttagagcctcaggaaaaggaagaaagtgccgaaaggttgaagaaaagaagcaggctttcctggaaggtcctggaagggggcgtccgcgggagctcacggggacagcccccccccaaagcccccagggctgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagctttaggctgaaagagagatttagaatgacagaatcatagaacggcctgggttgcaaggagcacagtgctcatccagatccaaccccctgctatgtgcagggtcatcaaccagcagcccagcgcgtcagagccacatccagcctggccttgaatgcctgcctgcagggatggggcatccacagcctccttgggcaacctgttcagtgcgtcaccaccctctggggaaaaactgcctcctcatatccaacccaaacctcccctgtctcagtgtaaagccattcccccttgtcctatcaagggggagtttgctgtgacattgttggtctggggtgacacatgtttgccaattcagtgctcacggagaggcagatcttgggataaggaagtgcaggacagcatggacgtggacatgcaggtgttgaggctctggacactccaagtcacagcgttcagaacagccttaaggtcaagaagataggatagaaggacaaagagcaagttaaaacccagcatggagaggagcacaaaaaggccacagacactgctggtccctgtgtctgagcctgcatgtttgatggtgtctggatgcaagcagaaggggtggatgagcttgcctggagagatacaggctgggtcgtaggactgggacaggcagctggagaattgccatgtagatgttcatacaatcgtcaaatcatgaaggctggaaaagccctccaagatccccaagaccaaccccaacccacccaccgtgccactggccatgtccctcagtgccacatccccacagttcttcatcacctccagggacggtgaccccccacctccgtgggcagctgtgccactgcagcaccgctctttggagaaggtaaatcttgctaaatccagcccgaccctcccctgggcacaacgttaaggccattatctctcatcctactccaggacggagtcagttgaggatgggggaatattgctgtgggtggcagatggccacatcccatgtccctcagtgccacatccccacagttcttcatcacctccagggacggtgaccccccacctccgtgggcagctgtgccactgcagcaccgctctttggagaaggtaaatcttgctaaatccagcccgaccctcccctgggcacaacgttaaggccattatctctcatcctactccaggacggagtcagttgaggatgggggaatattgctgtgggtggcagatggccacatcctggtcaaaggctataggcagaacgaagacacattcaatgtctgtgaggctgtgagtgaaaagcatttggaagaggcaggtgctaagctgatctctctggctccggccacagcatcctcagtgtggttgggattgcagatgtggacaaggccaggactgccacagctggcatagcgaggaggaggtacatggcacgtgcaggctctgctctgccttaatgacaacagggaacatccagttccccagcagcaccaggaggtacatggagggggcagctgagtggttttccatgatggaaaagccaccaagaagaatatcagcatccttggtctggctgctgttggtccaaggtgggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggactaagcttccactggattgtacaattacataaaataaaatatctttattttcattacatctgtgtgttggttttttgtgtgcgctact

SEQ ID NO: 29 - DNA, 15352 bp, synthetic construct
  source 1..15352: mol_type = other DNA
gtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatccggggagtgtgcagagtgagaggagcaagggaggcccttgggaacctcagcacttcaggggaaggtagaggtacagttgctggtgggaaaggcagagaagtagcaaagcaaaccaggcaaaagcagtgtcacagacgaccagggaggaaaaggacatgatcaaaatgttgagaaaagcagagaggtttgaaaatacaagaagcaaaaatgtccactagacttaaaaaccaggagaaaactggggggttcttgataaagcatcttagtaggatggtgagggtagaagccagggaagtgttggtgaggaagtgaagtcactgattacggactatgcttaaaagaatgtgggaatgaagggtggaagagagaaattagactgtagctagggagacataagcgatcagaggtagattctttctctcctgtgggagaatcttgcacgtatacacagcatgacgacagtgatggaagggctggcgaagcctcagggagactcttggaggtaaaccccatgaagggaggactttgtttcattcactgccgtgtccccagcacctggcacaatagcagacactcaatacatatttgtcaaatgtgggattttatcatttagaaactgcacctggctgtgagtaacaaaagtcagagaaaccgtgggtttcatttttctccccaggcagagtctggagctgggtcctccaagaggggtttggagcaccacaggtttcctcaagacccccaggctgccctgtgtttccctccttcatccccagcatatgcctgtcatctggtgacctccaaacacctgtgctgcctcctccagcacatccatgttgcaggcagggaccaggcaaagggcagaggggcctacttcaaaagaccatttccagaaaccccatcctatgacttctcctggtgtcttggttaccattgtgccataggctcaccctgtatgcatgggaggctgggccaggcattatgacttttagcaatattgcatagataagcatcaatctttgtcactgtgacgaagcctagtcactcagtgctaggcaaggttaatggaatgggttggtgtgtgcattattcttgaggtctttcttatgcttcatgttatacatttattaggacgtttaggcaacagggggataaaaatgaagaggagatgcatgctatgatctgaatgtttgcatcctccccaaaattcatatgttgaaatcttcatccccaagatgatggcattaggaggtggggcctttcggaggcaattaggtcatgactgggattagtgcccttgtaaaaccccagaaagccagcttgccgcttccaccatatgaagacacagagagaagatgccatctacgaatcaggaaatgagcccgcaccatgcaataaacctgctggagccttgatcttagacttcccagctgccagatctgtgggaaatagatttctgttgtttacccagcttatggtattttgttgtagcagccagagtgaactaagacagtgctgatctcgtattcttggagggaacccttagtctttagggaaagcaaagccaccatttggggcagggtgttctccaagtgctgccacatatgctgatgtggttaaactgcaaactatggtaaaaatgtggaggtctgtggaattgtcaatcaggaaaaagatataaaaagaagttaaagtcttcgtgcttctggaaggatatgtgccaaattgttaacattgattatccttgggtagagatgtggggaagtttgcagagacagttttgccttgtactttatataagtaaacagctactacttcgttgtcttaaaaaaaaaaaacagcctatgtgctcttcatgtgactcagaactacctaggcaatacgattaattgaattagtaaaattgagtgattatgaattttcaggaagtcattaatttaccacttctttattacatccacttctaacaggacttcaatataggggaatttgacttcaagataaaaagaccaaatttatttacccttttaaaaaaagacaacttaaaagcagacttgtcttacagaaccttccttagttggacatcgatgagtgtacagaaaatgcaatggataaaaagcttggtgatacaaagataaaaagtggggtcctgtccttaatgaacataccatttcatggagtatcaggtgtataaacaattataatcaatctgcttgttattctgataagatcatttactcacacatcaaatactgagtgcccaccacatgcccagcatacctagaagtcatccagtatgatttctgtctacatggagcatagagtcttacaggggagatagatgacaagtaaacaccagaataattaccaatggtgaagagcacaaggaaggaaacagaactcctaaagagagcgtggctgggcaggggtgagcaagaggcatagaaaaaggggcatctaaatctacttgggaggaagctgtttctcacataggtcatcatgttaggaatgagacttgagggatgagtagaagtttgccaggcaaagaaggaatgggggggaatagagagcagagctaggggcaggagacagctgacgtgtgagcagacataaaaagaagtccactgtggcagcagagaagcaggagagaaggcaagtgagggagccaggcaccagctcacagaggtcatgtgtgtcaaaacgtagtaatggccttctcttctggagacagtagggagccatggaagatgtttgagcagggaaagcgacatgactggattggcctgttgggtaactcagaccacaatgcattggaagggagggggctagaggcaaggggactggcaagaaggccagtcctttttctatgcctattttgatgaaatattctagaagggaagtgaacaaaggtagtcctagagaggaagaacaaaacagataggatacttccttagtatttgctcattcgacaatttatttttgcatatacactaaaaccttttttattattaaaacgttttattgtaggaaaaaagtatgaaagtagagtgaataataaaatgagctcccatggatctatcacccagcttcaactattatcaatatttggctgttcttgttttaactgttctccacctttttttcctgaagtttttttgaagcaaatcacagacaacatatcatttcaccatatgtacttccctctgtatctctaacatgtaagaacttgttttaacaaaatcaccatgctatgatcatacccaacaaaatttatcataatgtcttaataatacctaatacccatttcatgtccactttcccccaattgctacagctggtttgttcagatcagaatcaaaatccacctgtggccattttactgctatgtctctcaggtctcttttcatctctaataatctcaggggagacaggagggaggacgggcaggacttggggctaacttgcttatcgacacacagttttgcctacttgcttcctcccttcacacccactcttcttctcagccccacccttgtatggaaaaaacagaaattaaagtgctttgcccagcacccactgaagctatttcgaaggagtttgaagagtactcccggcaagacaaatgcctcggtccagtgctcaggtcaaagaggggagacgcttctcagtgatgtggtgtcaatagcagcttagttgttctttcctctggaaaattctacccatctgctttgtaactcccatacctaacaaggccttttatttcacaattagaaaataagcctgaaatatgaatgctgcctgagtgtacctacatttattctagagtttcagggtcaaaaagaatacaaggacctctgcatctacagccaagaggagaggggcaaagacacacagctacaaatgagaacctggctggtcaaagcctaactccacctgtttgtcagcactgatgcaagttaggtcagcccaatgatcatttaggagaactgtgctggcaaataaaaagcagaggcttttggtccccagatacttggatgagaattacaagtccagctggttaaaaggcacatgcccagtgctcacttcacacctactcaggaagcacacttgagttggaaaaccactgtctttacacttagaactcagtcctacatgactcctctaggatcagtgattccatcagttttgaaacatgaagcatgaagtcaaacaggacatgaccttggtttccagaaaaccagatgttcacatcagtctctggagcttggaggcagcacacctggggacttccacatcccctgccgaggtggcaaaagcaggagcagtggtgagttcacatgggctggggtttcctgaacactgctggcaattggagaatctgcaagggaacttctccgactcctaccagcagctgctttaaaataaaggtgatgtagctggtcaaatcctccatgagagagcagtgttgaatggaggaagagacacaacctgtctgaaaatggcacaaaggaagaaagatgtaaacaatgacgagaagactgcagtgtctacaaagctccgaggtgaacagatgggcaccccaggcccgcagcacttccttcagtctctgccagctgcactctgttttccttcctccaggaatcttgtttggtgtcactaaaacagcaattagaatcactttgaaatagtgatagtatttaatataactatgaaactatctgtgattgacaagtgcagcaaggagtcttggaatgagagcctttattttttcaattaaataaaagagttttttgtttctaaaagtaatcttgcagaaaagatcctgcgatcagaaagaaggagggggggagttttcaaacatataggagatcagactgtgcctatgtgtgtatatacctacaaacatatatatatttaaaaaattgttttactgtcaattacag

SEQ ID NO: 30 - DNA, 9177 bp, synthetic construct
  source 1..9177: mol_type = other DNA
aaaaactcttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggactaagcttgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctataaaataaaatatctttattttcattacatctgtgtgttggttttttgtgtgcgctact

SEQ ID NO: 31 - protein, 2215 aa, synthetic construct
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 32 - protein, 2273 aa, synthetic construct [sequence omitted]

SEQ ID NO: 33 - protein, 297 aa, synthetic construct
MSAVLQRFREKLPHKPYCTNDFAYGVRILPKNIAILARFIQQNQPHALYWLPFDVDRTGASIDWSDRNCPAPNITVKNPRNGHAHLLYALALPVRTAPDASASALRYAAAIERALCEKLGADVNYSGLICKNPCHPEWQEVEWREEPYTLDELADYLDLSASARRSVDKNYGLGRNYHLFEKVRKWAYRAIRQGWPVFSQWLDAVIQRVEMYNASLPVPLSPAECRAIGKSIAKYTHRKFSPEGFSAVQAARGRKGGTKSKRAAVPTSARSLKPWEALGISRATYYRKLKCDPDLAK

SEQ ID NO: 34 - DNA, 40 bp, synthetic construct
  source 1..40: mol_type = other DNA
agggcgctgttatctgataaggcttatctggtctcatttt

SEQ ID NO: 35 - DNA, 36 bp, synthetic construct
  source 1..36: mol_type = other DNA
agggcgctgttatctgataaggcttatctggtctca

SEQ ID NO: 36 - DNA, 33 bp, synthetic construct
  source 1..33: mol_type = other DNA
gcgctgttatctgataaggcttatctggtctca

SEQ ID NO: 37 - DNA, 27 bp, synthetic construct
  source 1..27: mol_type = other DNA
tgttatctgataaggcttatctggtct

SEQ ID NO: 38 - DNA, 28 bp, synthetic construct
  source 1..28: mol_type = other DNA
tgttatctgataaggcttatctggtctc

SEQ ID NO: 39 - DNA, 29 bp, synthetic construct
  source 1..29: mol_type = other DNA
tgttatctgataaggcttatctggtctca

SEQ ID NO: 40 - DNA, 30 bp, synthetic construct
  source 1..30: mol_type = other DNA
ctgttatctgataaggcttatctggtctca

SEQ ID NO: 41 - DNA, 31 bp, synthetic construct
  source 1..31: mol_type = other DNA
gctgttatctgataaggcttatctggtctca

SEQ ID NO: 42 - DNA, 32 bp, synthetic construct
  source 1..32: mol_type = other DNA
cgctgttatctgataaggcttatctggtctca

SEQ ID NO: 43 - DNA, 27 bp, synthetic construct
  source 1..27: mol_type = other DNA
tgttatctgataaggcttatctggtct

SEQ ID NO: 44 - DNA, 6822 bp, synthetic construct
  source 1..6822: mol_type = other DNA
atgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggactaa

SEQ ID NO: 45 - DNA, 6648 bp, synthetic construct
  source 1..6648: mol_type = other DNA
atggtcattctgcagcagggcgaccacgtgtggatggatctgagactgggccaagagttcgacgtgccaatcggcgccgtggtcaagctgtgtgattctggccaggtgcaggtcgtggacgacgaggataatgagcactggatcagccctcagaacgccacacacatcaagcctatgcaccccacatctgtgcacggcgtggaagatatgatccggctgggcgatctgaacgaggccggcatcctgagaaacctgctgatcagataccgggaccacctgatctacacctacaccggctctatcctggtggccgtgaatccctaccagctcctgagcatctacagccccgagcacatccggcagtacaccaacaagaaaatcggcgagatgcctcctcacatcttcgccattgccgacaactgctacttcaacatgaagcggaacagccgggaccagtgctgcatcatctctggcgaatctggcgccggaaagaccgagagcacaaagctgatcctgcagttcctggccgccatcagcggacagcactcttggattgagcagcaggtcctggaagccacacctattctggaagccttcggcaacgccaagaccatccggaacgacaacagcagcagattcggcaagtacatcgacatccacttcaacaagagaggcgccattgagggcgccaagatcgagcagtacctgctggaaaagtccagagtgtgcagacaggccctggacgagagaaactaccacgtgttctactgcatgctggaaggcatgagcgaggaccagaagaagaagctcggactcggccaggccagcgactacaattatctggccatgggcaactgcatcacatgcgagggcagagtggacagccaagagtacgccaacatccgcagcgccatgaaggtgctgatgttcaccgacaccgagaactgggagatcagcaaactgctggccgctatcctgcatctgggcaacctgcagtacgaggccagaaccttcgagaacctggatgcctgcgaggtgctgttctctcctagcctggctacagccgcctctctgctggaagtgaaccctcctgatctgatgagctgcctgaccagcagaaccctgatcaccagaggcgagacagtgtctacccctctgagcagagaacaggctctggatgtgcgggacgccttcgtgaagggcatctacggcagactgttcgtgtggatcgtggacaagatcaacgccgccatctacaagcctccaagccaggacgtgaagaacagcagaagatccatcggcctgctggacatcttcggcttcgagaatttcgccgtgaacagcttcgagcagctctgcatcaacttcgccaacgagcacctccagcagttcttcgtgcggcacgtgttcaagctggaacaagaggaatacgacctggaatccatcgactggctgcacatcgagttcaccgataaccaggacgccctggacatgatcgccaacaagcccatgaacatcatcagcctgatcgacgaggaaagcaagttccccaagggcaccgataccaccatgctgcacaagctgaacagccagcacaaactgaatgccaactacatcccgcctaagaacaaccacgagacacagttcggcatcaaccacttcgccggcatcgtgtactacgaaacccagggctttctggaaaagaaccgggacaccctgcacggcgacatcattcagctcgtgcacagcagccggaacaagttcatcaagcagatcttccaggccgacgtcgccatgggagccgagacaagaaagagaagccccacactgagcagccagttcaagcggagtctggaactgctgatgagaaccctgggagcctgccagcctttctttgtgcggtgcatcaagcccaacgagttcaagaaacccatgctgttcgaccggcacctgtgtgtgcggcagctcagatacagcggcatgatggaaaccatcaggattcggagagccggctatcccatccggtacagcttcgtggagttcgtcgagcggtacagagtgctgctgcctggcgtgaagcctgcctacaaacagggcgatctcagaggcacctgtcagagaatggccgaagccgtgctgggcacccatgacgattggcagatcggaaagacaaagatcttcctgaaggaccaccacgacatgctgctcgaggtggaaagagacaaggccatcaccgacagagtgatcctgctccagaaagtgatccggggcttcaaggacagaagcaatttcctgaagctgaagaatgccgccactctgatccagagacactggcggggacacaactgccggaagaactacggcctgatgaggctgggcttcctgagactgcaggccctgcacagaagcagaaagctgcaccagcagtacagactggcccggcagcggatcatccagtttcaagccagatgtcgggcctacctcgtgcgcaaggccttcagacatagactgtgggccgtgctgaccgtgcaggcctatgccagaggaatgattgcccgcagactgcaccagagactgagagccgagtatctgtggcggctggaagccgagaaaatgcggctggccgaggaagagaagctgcggaaagagatgagcgccaagaaggccaaagaagaggccgagcggaagcaccaagagagactggctcaactggccagagaggacgccgagagagagctgaaagagaaagaggccgccagacggaagaaagaactcctggaacagatggaacgggccagacacgagcccgtgaaccacagcgatatggtggataagatgttcggcttcctgggcacctctggcggactgcctggacaagaaggacaggcccctagcggctttgaggacctggaacgtgggagaagagaaatggtggaagaggatctggacgccgctctgcctctgcctgacgaggatgaagaagatctgagcgagtacaagttcgccaagtttgccgccacctactttcagggcaccaccacacacagctacaccagaaggcctctgaagcagcccctgctgtaccacgatgatgagggcgatcaactggcagccctggccgtgtggattaccatcctcagattcatgggcgacctgcctgagcctaagtaccacaccgccatgtctgacggctccgagaagatccccgtgatgaccaagatctacgagactctgggcaagaaaacctacaagcgcgagctgcaggctctccaaggcgaaggcgaagctcaactgcctgagggccagaaaaagtcctctgtgcgccacaaactggtgcacctgacactgaagaagaaaagcaagctgacagaggaagtgaccaagcggctgcacgatggcgagtctacagtgcagggcaacagcatgctcgaggacagacccaccagcaacctggaaaaactgcacttcatcatcggcaacggaatcctgcggcctgctctgagggatgagatctactgccagatctccaagcagctcacacacaaccccagcaagagcagctacgccagaggctggattctggtgtctctgtgcgtgggctgtttcgccccaagcgagaagttcgtgaagtacctgaggaacttcatccacggcggacctccaggctacgccccttactgtgaagagaggctgagaaggacctttgtgaacggcacccggacacagcctccatcctggctggaactccaggccaccaagagcaaaaagcccatcatgctgcccgtgacctttatggatggcaccacaaagaccctgctgaccgatagcgccaccaccgccaaagagctgtgtaacgccctggctgacaagattagcctgaaggatagattcggcttcagcctgtacattgccctgttcgacaaggtgtccagcctcggctctggctctgaccatgtgatggatgccatcagccagtgcgagcagtatgccaaagaacagggcgcccaagagaggaacgctccttggcggctgttctttcggaaagaggtgttcaccccttggcacagccccagcgaagataacgtggccaccaatctgatctaccagcaggttgtgcggggcgtgaagttcggcgagtacagatgcgaaaaagaggacgatctggccgagctggcctctcagcagtactttgtggactacggcagcgagatgatcctggaacggctgctgaatctggtgcccacctacattcccgatagagagatcaccccactgaaaaccctcgagaagtgggcccagctcgccattgctgcccacaagaaaggcatctatgcccagcggagaacagacgcccagaaagtcaaagaggatgtcgttagctacgcccggttcaagtggcctctgctgtttagcagattctacgaggcctacaagttcagcggccccagtctgcccaagaacgatgtgatcgtggctgtgaactggaccggcgtgtacttcgtggatgagcaagaacaagtgctgcttgagctgagcttccccgagatcatggccgtgtccagctccagagaatgcagagtgtggctgagcctgggctgtagcgatctgggatgtgccgctcctcattctggatgggctggactgacaccagccggaccttgtagcccttgttggtcttgcagaggggccaagacaacagcccctagctttaccctggccaccattaagggcgacgagtacaccttcaccagcagcaacgccgaggacatcagagatctggtcgtgaccttcctggaaggcctgcggaagcggagcaaatatgtggtggccctgcaggacaaccccaatcctgctggcgaggaatccggctttctgagctttgccaaaggcgacctgatcatcctggaccacgacaccggcgagcaagtgatgaatagcggctgggccaacggcatcaatgagcggacaaagcagcggggcgacttccctacagatagcgtgtacgtgatgcccaccgtgaccatgcctccaagggaaatcgtggccctggtcaccatgacacccgaccagagacaggatgttgtgcggctgctgcagctcaggacagccgaaccagaagtgcgggccaagccttacacactggaagagttcagctacgactacttccggcctcctccaaagcacaccctgtctagagtgatggtgtccaaggccagaggcaaggataggctgtggtcccacacaagagagcccctgaaacaggcactgctgaaaaagctgctgggcagcgaggaactgagccaagaagcctgtctggcctttatcgcagtgctgaagtacatgggcgattacccctccaagcggaccagatccgtgaacgaactgaccgaccagattttcgagggcccactgaaggccgagcctctgaaagatgaggcctacgtgcagattctgaaacagctcaccgacaaccacatccgctacagcgaggaacgcggatgggaactgctgtggctgtgtaccggactgttcccacctagcaacattctgctgccccacgtgcagcggtttctgcagtctagaaagcactgccctctggccatcgattgcctgcagaggctgcaaaaggccctgagaaatggctcccggaagtaccctcctcacctggtggaagtggaagccatccagcacaagaccacacagatctttcacaaggtctacttccccgacgacacagacgaggcctttgaggtggaatcctctaccaaggccaaggacttctgccagaatatcgccaccaggctgctgctgaagtccagcgaaggctttagcctgtttgtgaagatcgccgacaaagtgctgagcgtgcccgagaacgacttctttttcgattttgtgcgccatctgaccgactggattaagaaggctagacccatcaaggatggcatcgtgcccagcctgacctatcaggtgttctttatgaagaagctgtggacgaccaccgtgcctggcaaggatcctatggccgacagcatcttccactactaccaagagctgcccaagtacctgcggggctaccacaagtgtaccagagaagaggtcctgcagctcggagccctgatctatagagtgaagtttgaagaggacaagagctacttccctagcatccccaagctgctgcgcgaactggttccccaggatctgatccggcaagtgtcccctgatgactggaagcggtctatcgtggcctactttaacaagcacgccggcaagagtaaagaggaagccaagctggcctttctgaagctcatctttaagtggcctaccttcggctccgccttcttcgaagtgaagcagaccaccgagcctaacttccctgagattctgctgatcgccatcaacaaatacggcgtgtccctgatcgatcccaagacaaaggacatcctgacaacacaccccttcaccaaaatcagcaactggtccagcggcaacacctacttccacatcaccatcggcaatctcgtgcggggctctaagctgctgtgtgaaaccagcctgggatacaagatggacgacctgctgacaagctacatctcccagatgctgaccgccatgagcaaacagagaggctctcggagcggcaagtga